(19) United States
(12) Patent Application Publication
(10) Pub. No.: US 2017/0080417 A1
(43) Pub. Date: Mar. 23, 2017

(54) MICROFLUIDIC DEVICES THAT INCLUDE CHANNELS THAT ARE SLIDABLE RELATIVE TO EACH OTHER AND METHODS OF USE THEREOF

(71) Applicant: University of Limerick, Limerick (IE)

(72) Inventor: Mark Davies, Limerick (IE)

(73) Assignees: University of Limerick, Limerick (IE); University of Limerick, Limerick (IE)

(21) Appl. No.: 15/310,600

(22) PCT Filed: May 14, 2015
(86) PCT No.: PCT/IB2015/001509

§ 371 Date: Nov. 11, 2016

Related U.S. Application Data
(60) Provisional application No. 62/115,872, filed on Feb. 13, 2015.
(60) Provisional application No. 62/115,877, filed on Feb. 13, 2015.
(60) Provisional application No. 61/993,119, filed on May 14, 2014.

(51) Int. Cl. B01L 3/00; B01F 3/08; C12M 3/06; B01F 13/00; B01L 7/00; C12M 1/32

(52) U.S. Cl. CPC B01L 3/50857; B01L 3/5025; B01L 3/502738; B01L 3/502784; B01L 7/525; C12M 23/12; C12M 23/16; B01F 13/0083; B01F 3/0807; B01L 2200/027; B01L 2200/0647; B01L 2200/0673; B01L 2300/0829; B01L 2300/0864; B01L 2300/0874; B01L 2300/088; B01L 2300/165; B01L 2400/022; B01L 2400/0406; B01L 2400/0487; B01L 2400/065; B01L 2400/0688; B01L 2300/0663; B01L 2300/0654; B01F 2003/0834; B01F 2003/0842

(57) ABSTRACT

The invention generally relates to microfluidic devices that include channels that are slidable relative to each other and methods of use thereof. In certain embodiments, the invention provides a microfluidic device that includes a first channel having an open end, and a second channel having an open end. The first and second channels are slidable relative to each other such that when the open end of the first channel and the open end of the second channel are aligned with each other, fluid flows from the first channel into the second channel.

### RELATED APPLICATIONS

This application claims the benefit of and priority to U.S. provisional application No. 61/993,119 filed May 14, 2014; U.S. provisional application No. 62/115,872 filed Feb. 13, 2015; and U.S. provisional No. 62/115,877 filed Feb. 13, 2015, each of which is incorporated by reference.

### FIELD OF THE INVENTION

The invention generally relates to microfluidic devices that include channels that are slidable relative to each other and methods of use thereof.

### BACKGROUND

Microfluidic systems promise to be important in medical diagnostics and biotechnology research. Typically, components of such systems include networks of very small wells and channels, through which liquids can deliver and combine precisely-controlled aliquots of chemicals, cells, and molecules. The systems can be used for mixing chemicals to discover new compositions, the isolation and study of DNA, and even sequestering living cells. To accomplish the precise transfer, mixing, and accurate metering that's required, microfluidic chips and substrates require complex control machinery such as micro-valves and pumps built into the chip, as well as pneumatic actuators, electronic solenoids, pneumatic actuators, robotic controllers, and the complex computer programs and systems that are required to control those devices. All of the required computerized and electronic machinery is complex and failure-prone, while also requiring advanced software engineering to develop the control programs. Moreover, many specific research applications require the use of significant inputs of pressure, heat, or electrical currents to control fluid flow and those inputs can actually interfere with the materials being studied. Strong electrical currents used to drive a flow can disrupt a cell membrane, for example, while heat or pressure can adversely interfere with results of chemical reaction assays.

### SUMMARY

The invention generally relates to microfluidic devices that include channels that are slidable relative to each other and methods of use thereof. When an open end of a first channel and an open end of a second channel are aligned with each other, fluid flows from the first channel into the second channel. When the first and second channels are not aligned with each other, flow within the channels stops. In that manner, systems of the invention have no predefined geometry or arrangement of the microfluidic channels and a user can move the channels within the device as needed to suit the requirements of any assay. Accordingly, fluid flow and fluid mixing within the device is determined by the user as opposed to a predefined channel geometry that cannot be changed. Microfluidic devices of the invention do not require pumps or valves and flow can be achieved by gravitational force. Accordingly, systems and methods of the invention also avoid the problems associated with using electrodes to control flow within a microfluidic system. Consequently, systems and methods of the invention are particularly amendable to a wide range of cellular assays and have high throughput capabilities.

In certain aspects, the invention provides microfluidic devices that include a first channel having an open end, and a second channel having an open end. The first and second channels are slidable relative to each other such that when the open end of the first channel and the open end of the second channel are aligned with each other, fluid flows from the first channel into the second channel. The channels may slide in any direction relative to each other, e.g., horizontally, vertically, diagonally, etc. In certain embodiments, the channels are horizontally slideable relative to each other. Generally, systems of the invention are configured such that the open end of the first channel and the open end of the second channel are exposed to atmospheric pressure. In certain embodiments, the first and second channels are arranged in relation to each other such that an air gap exists when the open end of the first channel and the open end of the second channel are aligned with each other and fluid from the first channel bridges the air gap and enters the second channel. The first and second channels are configured such that when they are not aligned, fluid does not flow within the first and second channels. That can be achieved in numerous different ways, such as by adjusting length of the channels, internal diameter of the channels, viscosity of the fluid(s) within the channels, surface tension of the fluid(s) within the channels, and/or density of the fluid(s) within the channels.

Systems of the invention can be equipped with any type of flow driving mechanism, such as pumps. In particular embodiments, gravitational force is used to produce and control flow within the system. In such embodiments, the first and second channels are arranged such that gravity causes flow of fluid within the first and second channels when the open end of the first channel and the open end of the second channel are aligned with each other.

While described in the context of two channels for the sake of simplicity, the skilled artisan will recognize that the invention is not limited to two channels, and the invention encompasses systems designed with any number of channels, as will be described in embodiments below. For example, the system may further include at least one collection chamber downstream of the second channel. In another exemplary embodiment, the system further includes a third channel downstream of the second channel. The third channel includes an open end and the second and third channels are slidable relative to each other.

Another aspect of the invention provides methods for handling fluid. Such methods may involve providing a microfluidic system that includes a first channel having an open end, and a second channel having an open end, in which the first and second channels are slidable relative to each other such that when the open end of the first channel and the open end of the second channel are aligned with each other, fluid flows from the first channel into the second channel. A fluid is loaded into the first channel, and either the first or second channels are moved such that the open end of the first channel is aligned with the open end of the second channel, thereby causing at least a portion of the fluid to flow into the second channel. As discussed above, the system may include at least one collection chamber downstream of the second channel. In such embodiments, the method further involves aligning the open end of the second channel with an opening in the collection chamber, and flowing the portion of fluid from the second channel into the collection chamber.

Systems and methods of the invention can be loaded with any fluid(s) (e.g., liquid(s)).

The fluid may be a single phase fluid. Alternatively the fluid may include two phases, such as a fluid that includes droplets that are immiscible with the fluid. Such an exemplary two phase fluid is an oil that includes droplets of an aqueous fluid. In certain embodiments, the oil includes a surfactant.

Systems and methods of the invention can be used to partition a fluid into droplets and/or can be used to cause droplets to merge with each other. All process can be repeated numerous times as fluid flows through the system.

### Orthogonal Slidable Channels

The invention provides microfluidic systems in which a liquid can be held within a reservoir and then transferred into a channel simply by bringing the channel in proximity to the liquid. Initially, the fluid is held within the reservoir by surface tension even where the reservoir is open at both a top and bottom end. A substrate defining the channel is brought into contact with a surface of the liquid exposed at the open bottom of the reservoir. When a portion of the substrate, e.g., a portion defining part of an edge of the channel, makes contact with the exposed surface of the liquid, the surface tension is broken and the liquid flows out of the reservoir and into the channel. Thus aliquots of liquid can be transferred or combined through simple mechanical motion among components of a microfluidic device. The described devices provide good control over the volumes of liquid that are transferred, and even provide a mechanism by which water-in-oil droplets can be created and moved from chip to chip. The liquid transfer by the described devices can be precise and very rapid, providing a very simple mechanism by which reagents and compounds can be held and combined. No extrinsic inputs that would influence chemical reactions or interfere with the materials being studied need to be used. Thus the described methods and systems provide simple, rapid, and effective microfluidic systems. The invention generally relates to microfluidic channels and components that are slidable relative to each other, allowing for controlled fluid direction, diversion, or collection. Because there is no predetermined geometry, the invention allows for a custom designed microfluidic device. The microfluidic components are interchangeable and can be assembled within a three-dimensional space using vertical and horizontal microfluidic channels and compartments.

Additionally, the microfluidic components can be positioned on robotic stages to control the flow and timing of reactions within the assay. The robotic stages can be controlled manually or automatically, allowing for microfluidic channels to slide and align based upon a computer program. Materials of the assay are held in various microfluidic channels and vessels. The ability to compartmentalize the materials of an assay within various microfluidic components of the invention and then program the alignment of the components at specific time points allows the user to reduce human error at various stages of the assay. Therefore, the user can design the positioning of the microfluidic components and also control the flow of fluids through the device to perform the assay in a substantially closed, single system.

The invention generally relates to microfluidic devices that include microfluidic channels that are slidable relative to each other. In an aspect of the invention, microfluidic devices of the invention include orthogonally positioned channels which are slidable relative to each other, and methods of use thereof. When an open end of a first channel and an open second channel are aligned with each other, fluid flows from the first channel into the open channel. When the first and open channels are not aligned with each other, flow within the channels stops. In that manner, systems of the invention have no predefined geometry or arrangement of the microfluidic channels and a user can move the channels within the device as needed to suit the requirements of any assay. Accordingly, because the channels can be positioned orthogonally, assays in the vertical and horizontal direction can accomplished.

Fluid flow and fluid mixing within the device is determined by the user as opposed to a predefined channel geometry that cannot be changed. Microfluidic devices of the invention do not require pumps or valves and flow can be achieved by gravitational force. Accordingly, systems and methods of the invention avoid the problems associated with using electrodes and other devices to control flow within a microfluidic system. Consequently, systems and methods of the invention are particularly amendable to a wide range of cellular assays and have high throughput capabilities.

In certain aspects, the invention provides microfluidic devices that include a first channel having an open end, and a second channel that is an open channel. The first channel and the open channel are slidable relative to each other such that when the open end of the first channel and the open channel are aligned with each other, fluid flows from the first channel into the open channel. Since the open channel is open along the top, the first channel can align anywhere along the open channel. The channels may slide in any direction relative to each other (e.g., horizontally, vertically, diagonally, etc.). Generally, systems of the invention are configured such that the open end of the first channel and the open channel are exposed to atmospheric pressure. In certain embodiments, the first and open channels are arranged in relation to each other such that an air gap exists when the open end of the first channel and the open channel are aligned with each other and fluid from the first channel bridges the air gap and enters the open channel. The first and open channels are configured such that when they are not aligned, fluid does not flow within the first and second channels. In alternative embodiments, the first and open channels are configured such that when they are not aligned, fluid does not flow within the first, but may flow in the open channel.

Systems of the invention can be equipped with any type of flow driving mechanism, such as pumps. In particular embodiments, gravitational force is used to produce and control flow within the system. In such embodiments, the first and second channels are arranged such that gravity causes flow of fluid within the first and second channels when the open end of the first channel and the open end of the second channel are aligned with each other.

While described in the context of two channels for the sake of simplicity, the invention is not limited to two channels. Rather, the invention encompasses systems designed with any number of channels, as will be described in embodiments below. Microfluidic devices of the invention may include a plurality of microfluidic channels and a plurality of open channels. The system may further include at least one collection chamber downstream of the second channel.

Another aspect of the invention provides methods for handling fluid. Such methods may involve providing a microfluidic system that includes a first channel having an open end, and an open channel, in which the first and open channels are slidable relative to each other. When the open end of the first channel and the open channel are aligned with each other, fluid flows from the first channel into the open channel. A fluid is loaded into the first channel, and either the first or open channels are moved such that the open end of the first channel is aligned with the open channel, thereby causing at least a portion of the fluid to flow into the open channel. As discussed above, the system may include at least one collection chamber downstream. In such embodiments, the method further involves aligning the open channel with an opening in the collection chamber, and flowing the portion of fluid from the open channel into the collection chamber.

Systems and methods of the invention can be loaded with any fluid(s) (e.g., liquid(s)). The fluid may be a single phase fluid. Alternatively the fluid may include two phases, such as a fluid that includes droplets that are immiscible with the fluid. Such an exemplary two phase fluid is an oil that includes droplets of an aqueous fluid. In certain embodiments, the oil includes a surfactant.

Systems and methods of the invention can be used to partition a fluid into droplets and/or can be used to cause droplets to merge with each other. All processes can be repeated numerous times as fluid flows through the system.

### BRIEF DESCRIPTION OF THE DRAWINGS

FIGS. 1A-1B show an embodiment of a system of the invention.

FIGS. 2A-2C show another embodiment of a system of the invention having a greater number of channels than shown in the embodiment of FIGS. 1A-1B. FIGS. 2A and 2B show microfluidic channels etched within a substrate, in which the substrates are aligned and misaligned. FIG. 2C shows a cross section of the substrate.

FIGS. 3A-3C depict a series of steps of sliding the microfluidic channels to mix the contents of at least two microfluidic channels.

FIGS. 4A-4B depict a multichannel system of the invention, including arrangements of microfluidic channels for forming droplets.

FIGS. 5A-5E depict a series of steps in which alignment of microfluidic channels results in the mixing of droplets.

FIG. 6 shows a multichannel system of the invention in which droplets are diverted to microfluidic channels, waste chambers, incubation chambers, etc.

FIG. 7A-7C shows a system of the invention for a cellular assay for compound screening.

FIG. 8 depicts a branched microfluidic channel.

FIG. 9 depicts a circular microfluidic channel.

FIG. 10 depicts a multichannel system for assaying samples in droplets.

FIG. 11 depicts a series of microfluidic channels.

FIGS. 12A-12E depict microfluidic channels for transportation.

FIG. 13 depicts a schematic of a microfluidic channel system.

FIGS. 14A-14C show an embodiment of a system of the invention.

FIGS. 15A-15D show another embodiment of a system of the invention.

FIGS. 16A-16C depict open channels in a substrate.

FIGS. 17A-17B depict alternate nonlinear forms of the channels.

FIG. 18 shows an embodiment of the invention.

FIGS. 19A-19C depict a multi-channel system.

FIGS. 20A-20C depict a multi-channel system.

FIGS. 21A-21C depict a multichannel system with a shuttle.

FIGS. 22A-22C depict alternate embodiments.

FIGS. 23A-23B show a multichannel system of the invention.

FIGS. 24A-24C depict alternate embodiments.

FIG. 25 depicts a multi-channel system.

FIG. 26 depicts a multi-channel system.

FIGS. 27A-27D depict channel systems.

FIG. 28 depicts a schematic of the channels with variables.

FIG. 29 depicts a graph of dispensing time versus droplet volume.

FIGS. 30A and 30B depict a system of the invention on a mechanical subsystem.

### DETAILED DESCRIPTION

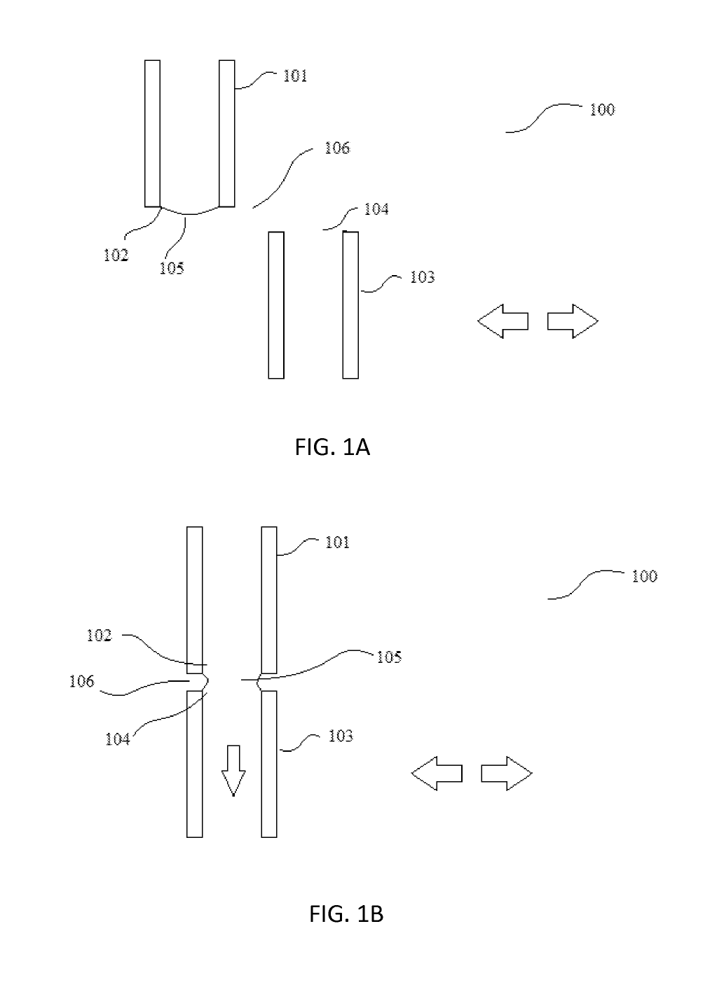

The invention generally relates to microfluidic devices that include channels that are slidable relative to each other and methods of use thereof. FIGS. 1A-1B show an exemplary embodiment of a microfluidic system 100 of the invention. FIGS. 1A-1B are described in the context of two channels for the sake of simplicity. However, the skilled artisan will recognize that the invention is not limited to two channels, and the invention encompasses systems designed with any number of channels, as will be described in additional embodiments below. Microfluidic system 100 includes a first channel 101 having an open end 102, and a second channel 103 having an open end 104. The first and second channels are slidable relative to each other such that when the open end 102 of the first channel 101 and the open end 104 of the second channel 103 are aligned with each other, fluid 105 flows from the first channel 101 into the second channel 103 (FIG. 1B, flow shown by large downward pointing arrow within channel 103). When the first channel 101 and the second channel 103 are not aligned, fluid 105 does not flow within the first channel 101 and the second channel 103 (FIG. 1A). Alignment includes complete alignment, partial alignment, and misalignment. In complete alignment, the center axes of two microfluidic channels are aligned. In partial alignment, the center axes are not aligned, however, there is partial overlap of the first and second channels such that the distance between the center axes is sufficiently small so that flow between the two microfluidic channels occurs. In complete misalignment, there is no overlap between the channels and the distance between the center axes is sufficiently great so that flow between the two microfluidic channels does not occur. In the present invention, alignment is meant to encompass both complete and partial alignment. The device of the invention flows fluid between two microfluidic channels even in the cases of partial alignment.

The channels may slide in any direction relative to each other, e.g., horizontally, vertically, diagonally, etc. In certain embodiments, the first channel 101 and the second channel 103 are horizontally slideable relative to each other as shown in FIGS. 1A-1B (horizontal arrows). FIGS. 1A-1B show second channel 103 being slideable relative to first channel 101, which remains stationary. However the invention is not limited to such a configuration. In other embodiments, it may be first channel 101 that is slidable relative to second channel 103, which remains stationary. In another embodiment, both the first channel 101 and the second channel 103 are slidable, that is, neither channel remains stationary and both channels are movable.

In certain embodiments, the open end 102 of the first channel 101 and the open end 104 of the second channel 103 are exposed to atmospheric pressure. In such embodiments, the first channel 101 and second channel 103 may be arranged in relation to each other such that an air gap 106 exists between the channels. As shown in FIGS. 1A-1B, when the open end 102 of the first channel 101 and the open end 104 of the second channel 103 are aligned with each other, fluid 105 from the first channel 101 bridges the air gap 106 and enters the second channel 103.

In an aspect of the invention, the air gap may comprise any known gas, at any temperature and pressure. The air gap may be at atmospheric pressure and be comprised of air. However, the air gap is not limited to atmospheric pressure or air. In some embodiments, the devices of the present invention may be completely or partially enclosed within a chamber and the chamber may be filled with a gas other than air. The pressure can be above or below atmospheric pressure and the temperature can be at, above, or below room temperature, which is about 37 degrees Celsius. Systems of the invention can be equipped with any type of flow driving mechanism, such as pumps. In particular embodiments, gravitational force is used to produce and control flow within the system. As shown in FIGS. 1A-1B, the first channel 101 and the second channel 103 are arranged (e.g., arranged vertically) such that gravity causes flow of fluid 105 within the first channel 101 and second channel 103 when the open end 102 of the first channel 101 and the open end 104 of the second channel 103 are aligned with each other.

The first channel 101 and second channel 103 may be configured such that when they are not aligned, fluid 105 does not flow within the first channel 101 and/or second channel 103. That can be achieved in numerous different ways, such as by adjusting length of the channels, internal diameter of the channels, viscosity of the fluid(s) within the channels, surface tension of the fluid(s) within the channels, and/or density of the fluid(s) within the channels.

### Channels

The microfluidic systems of the present invention include channels that form the boundary for a fluid. A channel generally refers to a feature on or in the system (sometimes on or in a substrate) that at least partially directs the flow of a fluid. In some cases, the channel may be formed, at least in part, by a single component, e.g., an etched substrate or molded unit. The channel can have any cross-sectional shape, for example, circular, oval, triangular, irregular, square or rectangular (having any aspect ratio), or the like, and can be covered or uncovered (i.e., open to the external environment surrounding the channel). In embodiments in which the channel is completely covered, at least one portion of the channel can have a cross-section that is completely enclosed, and/or the entire channel may be completely enclosed along its entire length with the exception of its inlet and outlet.

An open channel generally will include characteristics that facilitate control over fluid transport, e.g., structural characteristics (an elongated indentation) and/or physical or chemical characteristics (hydrophobicity vs. hydrophilicity) and/or other characteristics that can exert a force (e.g., a containing force) on a fluid. The fluid within the channel may partially or completely fill the channel. In some cases the fluid may be held or confined within the channel or a portion of the channel in some fashion, for example, using surface tension (e.g., such that the fluid is held within the channel within a meniscus, such as a concave or convex meniscus). In an article or substrate, some (or all) of the channels may be of a particular size or less, for example, having a largest dimension perpendicular to fluid flow of less than about 5 mm, less than about 2 mm, less than about 1 mm, less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm or less in some cases. Of course, in some cases, larger channels, tubes, etc. can be used to store fluids in bulk and/or deliver a fluid to the channel. In one embodiment, the channel is a capillary.

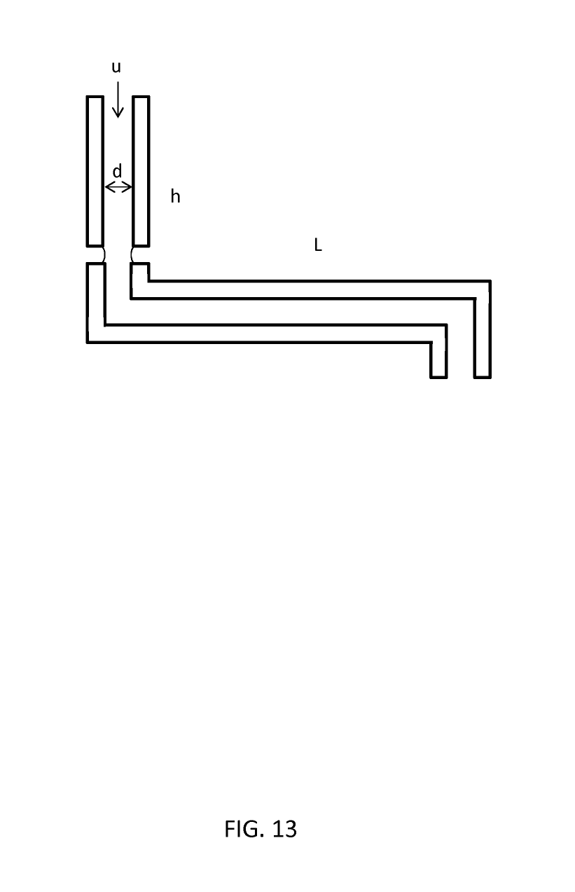

The microfluidic channels of the invention are configured such that liquid is retained within the microfluidic channel when it is completely out of alignment with another microfluidic channel (e.g., no overlap between open ends of channels). Liquid may be retained within the microfluidic channel due to surface tension. The flow in a microfluidic channel system, as shown in FIG. 13, with a height of h, an internal diameter of d, a length of L, a fluid velocity of u, a fluid density of ρ, gravitation force of g, fluid viscosity of μ, and surface tension of γ, can be represented by the equation:

$$2 ρ gh = \frac{γμ u ( 2 h + L )}{{( \frac{d}{2} )}^{2}}$$

or, rearranged as:

$$u = \frac{ρ {ghd}^{2}}{2 γμ ( 2 h + L )}$$

When fluid does not flow in the system, at maximum height, the equation becomes h=4γ/dρg.

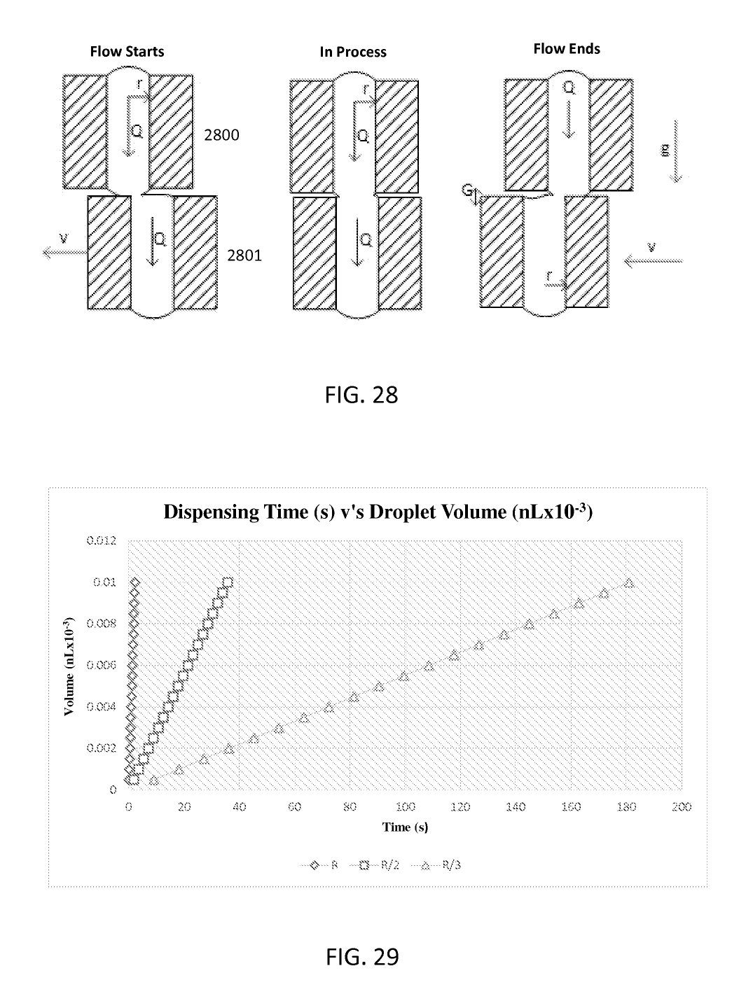

The volume of fluid that flows from one channel to another channel depends on the amount of time that the channels are aligned. As shown in FIG. 28, two channels 2800 and 2801 are aligned. Q is the flow rate in each channel, v is the velocity of the sliding channel, and r is the radius of the channel. Time when flowing is equal to nr/v, where n is the fraction of the lateral distance. As channel 2801 moves at a velocity relative to channel 2800, a volume of fluid flows from channel 2800 into channel 2801. G is the gap between the channels, and g is the force of gravity. The following equations denote the time required to dispense a volume, V from one channel to another channel. R is the resistance, P is the pressure, and u is the velocity.

$$R = \frac{8 µ L}{π {r}^{4}} , Δ P = QR , Δ P = ρ gh \\ ∴ ρ gh = Q ( \frac{8 µ L}{π {r}^{4}} ) = π {r}^{2} u ( \frac{8 µ L}{π {r}^{4}} ) \\ ∴ ρ gh = u ( \frac{8 µ L}{{r}^{2}} )$$

$$Qt = V where V = volume dispensed . \\ ρ gh = \frac{V}{t} ( \frac{8 µ L}{π {r}^{4}} )$$

For a given volume displaced we look to minimise time t.

$$t = \frac{V}{ρ gh} ( \frac{2 µ L}{π {r}^{4}} ) \\ ∴ t = \frac{8 µ LV}{ρ gh π {r}^{4}}$$

$$h = L for vertical channels \\ ∴ t = \frac{8 µ V}{ρ gh π {r}^{4}}$$

This equation denotes the time required to dispense a volume, V.

FIG. 29 is a graph showing dispensing time (t) versus droplet volumes produced (nL) for varying vena contracta. The vena contracta means that average r is constantly changing, and can be averaged to r/2.

The dimensions of the channel may be chosen such that fluid is able to freely flow through the channel when channels are aligned and will not flow when channels are out of alignment with each other. The dimensions of the channel may also be chosen, for example, to allow a certain volumetric or linear flow rate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, etc.

The channels of the device of the present invention can be of any geometry as described. However, the channels of the device can comprise a specific geometry such that the contents of the channel are manipulated, e.g., sorted, mixed, prevent clogging, etc. For example, for channels that are configured to carry droplets, the channels of the device may preferably be square, with a diameter between about 2 microns and 1 mm. This geometry facilitates an orderly flow of droplets in the channels.

To prevent material (e.g., cells and other particles or molecules) from adhering to the sides of the channels, the channels (and coverslip, if used) may have a coating which minimizes adhesion. Such a coating may be intrinsic to the material from which the device is manufactured, or it may be applied after the structural aspects of the channels have been microfabricated. “TEFLON” (polymer, commercially available from DuPont, Inc.) is an example of a coating that has suitable surface properties. The surface of the channels of the microfluidic system can be coated with any anti-wetting or blocking agent for the dispersed phase. The channel can be coated with any protein to prevent adhesion of the biological/chemical sample. For example, in one embodiment the channels are coated with BSA, PEG-silane and/or fluorosilane. For example, 5 mg/ml BSA is sufficient to prevent attachment and prevent clogging. In another embodiment, the channels can be coated with a cyclized transparent optical polymer obtained by copolymerization of perfluoro (alkenyl vinyl ethers), such as the type sold by Asahi Glass Co. under the trademark Cytop. In such an embodiment, the coating is applied from a 0.1-0.5 wt % solution of Cytop CTL-809M in CT-Solv 180. This solution can be injected into the channels of a microfluidic device via a plastic syringe. The device can then be heated to about 90° C. for 2 hours, followed by heating at 200° C. for an additional 2 hours. In another embodiment, the channels can be coated with a hydrophobic coating of the type sold by PPG Industries, Inc. under the trademark Aquapel (e.g., perfluoroalkylalkylsilane surface treatment of plastic and coated plastic substrate surfaces in conjunction with the use of a silica primer layer) and disclosed in U.S. Pat. No. 5,523,162, which patent is hereby incorporated by reference. By fluorinating the surfaces of the channels, the continuous phase preferentially wets the channels and allows for the stable generation and movement of droplets through the device. The low surface tension of the channel walls thereby minimizes the accumulation of channel clogging particulates.

The surface of the channels in the microfluidic device can be also fluorinated to prevent undesired wetting behaviors. For example, a microfluidic device can be placed in a polycarbonate dessicator with an open bottle of (tridecafluoro-1,1,2,2-tetrahydrooctyl)trichlorosilane. The dessicator is evacuated for 5 minutes, and then sealed for 20-40 minutes. The dessicator is then backfilled with air and removed. This approach uses a simple diffusion mechanism to enable facile infiltration of channels of the microfluidic device with the fluorosilane and can be readily scaled up for simultaneous device fluorination.

Microfluidic systems of the invention can take any form as long as they include one or more microfluidic channels. In certain embodiments, the channels are within one or more substrates. If the channels are within a single substrate, the substrate has different slidable portions so that the channels are slidable relative to each other within the substrate. In other embodiments, the channels can span more than one substrate, and the separate substrates are movable relative to each other.

The substrates may be formed by known methods in the art. The substrates may be formed by several different types of materials, such as silicon, plastic, quartz, glass, plastic, or other suitable materials. Also, it should be appreciated that the size, shape and complexity of the microfluidic channels and structures that can be used in the microfluidic device depends on the materials used and the fabrication processes available for those materials. Typical system fabrication includes making trenches in a conducting material (silicon) or in a non-conducting substrate (e.g., glass or plastic) and converting them to channels by bonding a cover plate to the substrate. See for example, U.S. Pat. No. 6,210,986. In addition, for example, U.S. Pat. No. 5,885,470 teaches a microfluidic device having application in chemistry, biotechnology, and molecular biology that provides precise control of fluids by forming various grooves or channels and chambers in a polymeric substrate. The process of forming microfluidic channels in a substrate can include wet chemical etching, photolithographic techniques, controlled vapor deposition, and laser drilling into a substrate.

Alternative techniques for constructing microfluidic channels may be employed in the fabrication of the device of the invention. For example, in M. Stjernstrom and J. Roeraade, Method for Fabrication of Microfluidic Systems in Glass, J. Micromechanics and Microenginneering, 8, 33-38, 1998, walls are formed that define the channels rather than simply forming trenches in the substrate. The silicon nitride channels and are formed by conformal coating etched features in a silicon wafer with deposited silicon nitride. The silicon nitride channels are bonded to the glass substrate by an intermediate thermal oxide layer grown on the surface of the silicon nitride. The silicon wafer is etched away leaving silicon nitride channels on the surface of the glass substrate. An electrically insulating material can be applied to the substrate to support the silicon nitride structures.

Microfluidic channels of the present invention may be formed by injection molding. In this process of preparing a microfluidic device by injection molding, in some methods, a polymeric material is injected into an injection molding mold or mold insert and the polymeric material is cured in the model to form the substrate of the microfluidic device. However, an injection molding mold or mold insert may be prepared from materials such as metal, silicon, ceramic, glass, quartz, sapphire and polymeric materials. The forming of the negative impression of the channel architecture may be achieved by techniques such as photolithographic etching, stereolithographic etching, chemical etching, reactive ion etching, laser machining, rapid prototyping, ink jet printing and electroformation. With electroformation, the injection molding mold or mold insert is formed as the negative impression of the channel architecture by electroforming metal, and the metal mold is polished, preferably polished to a mirror finish. The devices of the present invention may be manufactured by injection molding using any suitable thermoplastic, for example, polycyclic olefin polyethylene co-polymers, poly methyl methacrylate (PMMA), polycarbonate, polyalkanes and polystyrenes. The microfluidic devices can be fabricated in accordance with the invention by compression molding and casting on a wide range of polymers. Polymers preferred for microfluidic devices are low melt viscosity polymers with minimal amount of leachable additives, for example, polycyclic olefin polyethylene co-polymers.

Ink jet technology may be applied in fabricating the microfluidic devices directly, or in fabricating the molds used making microfluidic devices by injection molding Ink-jet printing technology provides the desired microfluidic features to be printed directly on a substrate such as glass, ceramics, silicon, polymers or any organic, inorganic or hybrid materials that form a flat surface for the printing of features. A negative of the microfluidic features may be made by conventional electroplating with copper or nickel, or any other metals over the device made via printing technology. The materials forming the microfluidic features may be organic, inorganic, or a blend of organic and inorganic materials. See for example, WIPO Patent Application WO/2002/063288.

Microfluidic channels may be formed by the sealing of numerous layers. For example, the deposition of a thin film on one of two glass substrates followed by an anodic (also frequently called electrostatic) bonding process. This metallic or semiconducting layer can be used as an intermediate layer. An example of this method is described in the article “Glass-to-glass anodic bonding with standard IC-technology thin films as intermediate layers,” by A. Berthold et. al., Sensors & Actuators A Vol. 82, 2000, pp. 224-228. Additionally, anodic bonding of a glass to a silicon substrate is a method that can be employed in the fabrication of the device of the invention. See for example U.S. Pat. No. 3,397,278. Bonding between two insulator substrates can be accomplished with direct anodic bonding. See for example U.S. Patent Number U.S. Pat. No. 3,506,424. This method comprises the evaporation of a thin layer of SiO on thin film circuitry, present on a substrate, and subsequent anodic bonding of a glass foil. Glass layers may be bonded by thermal glass-to-glass bonding, which consists in heating both substrates to a temperature at which melting starts to occur, or at least to a temperature at which the glass starts to soften, e.g. at 550 degrees C., and pressing the substrates together, by which a bond is formed. Bonding of two glass substrates through an intermediate layer of a low-melting-point material, or through an intermediate layer which solidifies from a solution during heat treatment is known in the art. It should be appreciated that known techniques can be employed for sealing or joining layers of materials in forming microfluidic devices of the invention.

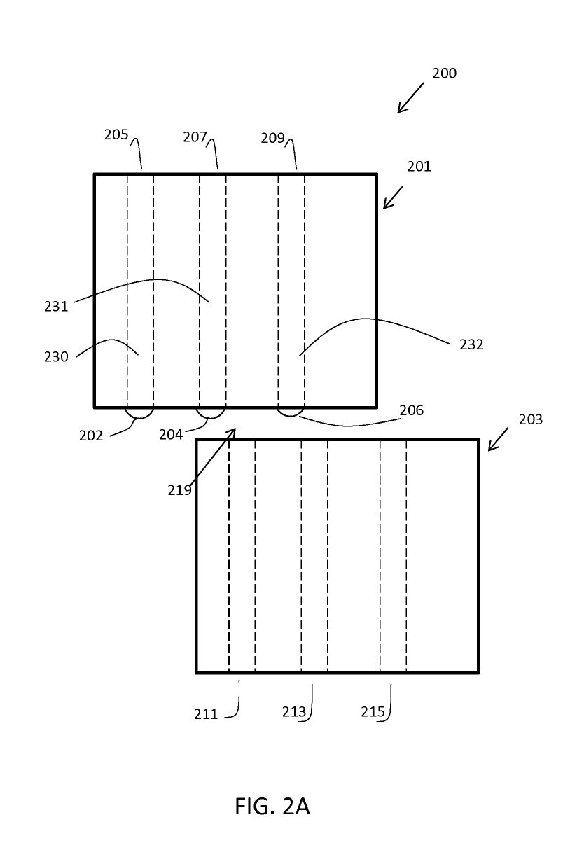

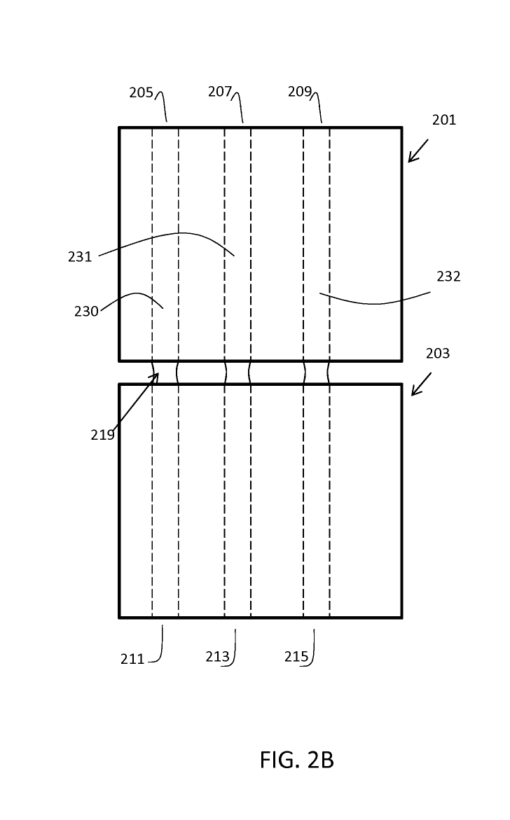

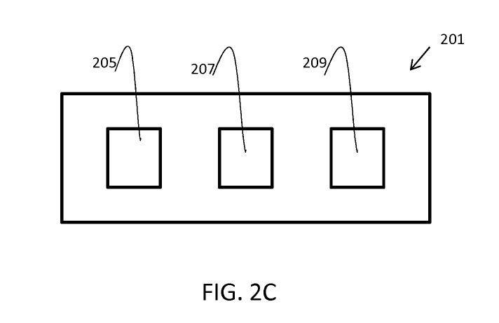

Microfluidic devices for analysis or synthesis of biological and chemical species can be fabricated from two flat electrically insulating glass substrates, with one substrate containing an etched microfluidic channel and drilled or etched access-holes. The glass plates are bonded together so that the microfluidic channel in one substrate forms together with the second glass substrate a microfluidic channel. In this arrangement, as shown in FIG. 2C, the walls of the microfluidic channel are bound by the material comprising the substrate. Ample illustrative examples of such devices can be found in literature, D. J. Harrison and co-workers, in: “Capillary electrophoresis and sample injection systems integrated on a planar glass chip,” Analytical Chemistry vol. 64, 1 Sep. 1992, p. 1926, which describes a micromachined glass chip, which employs electrokinetic and electroosmotic principles for sample preparation and liquid propulsion, and demonstrates electrophoresis on the chip.

Various layers can be formed to define the walls of the microfluidic channels. For example, the substrate may comprise various glass layers, and may include an elastomeric layer, wherein two glass layers interfaced to form one or more microfluidic channels. An elastomeric layer may be positioned between glass layers to form one or more microfluidic channels. For example, layers may include borosilicate glasses, pyrex, borofloat glass, Corning 1737, Corning Eagle 2000, silicon acrylic, polycarbonate, liquid crystal polymer, polymethylmethoxyacrylate (PMMA), Zeonor, polyolefin, polystyrene, polypropylene, and polythiols. Depending on the choice of the material different fabrication techniques may also be used.

Systems of the invention may be made out of plastic, such as polystyrene, using a hot embossing technique. In that process, the microfluidic channels are embossed into the plastic to create the bottom surface. A top layer may then be bonded to the bottom layer. In an alternative fabrication method, the use of epoxy casting techniques to create the microfluidic channels through the use of UV or temperature curable epoxy on a master that has the negative replica of the intended structure can be employed. Laser or other types of micromachining approaches may also be utilized to create microfluidic channels.

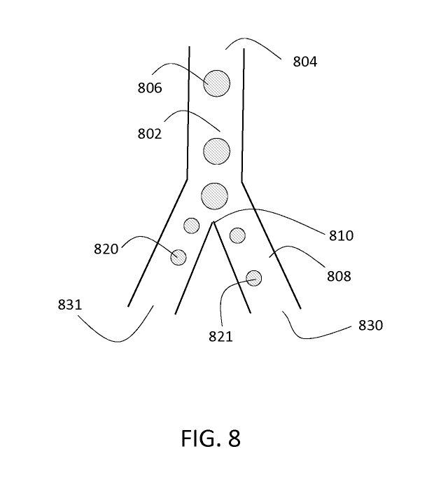

Microfluidic channels of the invention may be linear or nonlinear. Microfluidic channels may be curved, or have other nonlinear configurations. Microfluidic channels of the invention may be branched, containing one or multiple branches within the channel. For example, as shown in FIG. 8, a microfluidic channel 804 can be configured to have a branched portion 808. In this embodiment, the microfluidic channel 804 contains droplets 806 within an immiscible fluid 802. An obstacle 810 is positioned so droplets 806 contact the obstacle and split into at least two different parts 820 and 821. It should be appreciated that the open ends of the microfluidic channel 831 and 830 are able to align with other microfluidic channels or chambers (discussed below). The ability to split droplets is useful for certain assays, such as assays that involve culturing cells within droplets. In addition, a microfluidic channel cross sectional diameter may remain constant, or the diameter may wide or narrow. In an aspect of the invention, the channel diameter may widen and narrow to coalesce droplets.

FIGS. 2A-2C show another exemplary embodiment of a system 200 of the invention in which the channels are formed in multiple substrates. As shown in FIGS. 2A-2C, the microfluidic channels are housed within two different substrates that move relative to one another. Either substrate 201 or 203 may be stationary, in which only one of the substrates moves relative to the other. In other embodiments, both substrates 201 and 203 may be slidable or moveable. As discussed above, the substrates may be formed from any materials and by any techniques discussed above. Substrate 201 contains microfluidic channels 205, 207, and 209, and substrate 203 contains microfluidic channels 211, 213 and 215. In this specific embodiment, the microfluidic channels are open ended at each end is open to atmospheric pressure. For example, microfluidic channel 205 is open to the atmosphere at 202.

As shown in FIG. 2A, substrates 201 and 203 are positioned so that the microfluidic channels are not aligned. However, as discussed above, the substrates are moveable or slidable and therefore can be positioned so that microfluidic channels align. The substrates can be moved in any direction relative to each other, such as vertically or horizontally. FIG. 2A shows substrates 201 and 203 in which the microfluidic channels are not aligned. In that position, fluid within microfluidic channels 205, 207, and 209 is not able to flow. For example, fluid 230 housed in microfluidic channel 205 is open to atmospheric pressure, however, due to channel dimensions and forces such as surface tension, fluid 230 remains in microfluidic channel 205. Fluids 230, 231, and 232 may be the same or different. Substrate 201 and/or 203 can slide to align the microfluidic channels. Either substrate 201 or 203 can slide, or both substrates 201 or 203 can slide to align the microfluidic channels. As shown in FIG. 2B, substrates 201 and 203 are positioned so that the microfluidic channels are aligned. The substrates do not need to align so that the surfaces of the substrate are flush. The substrates may be aligned to create an air gap 219. As shown in FIG. 2B, alignment of the microfluidic channels allows fluid to bridge the air gap 219 and flow from substrate 201 to substrate 203. For example, as shown in FIG. 2B, microfluidic channel 205 is aligned with microfluidic channel 211 thereby creating an arrangement in which fluid bridges the air gap 219 and flows between microfluidic channels 205 and 211.

FIG. 2C shows a cross sectional representation of substrate 201 depicting that the walls of microfluidic channels 205, 207, and 209 are formed by substrate 201. It should be appreciated that the microfluidic channels may have any cross sectional geometry, as discussed above.

### Multichannel Systems

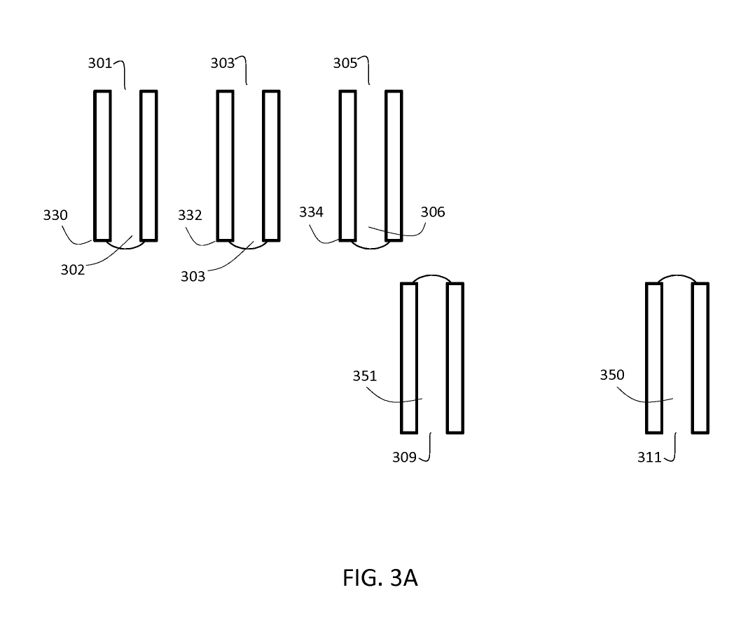

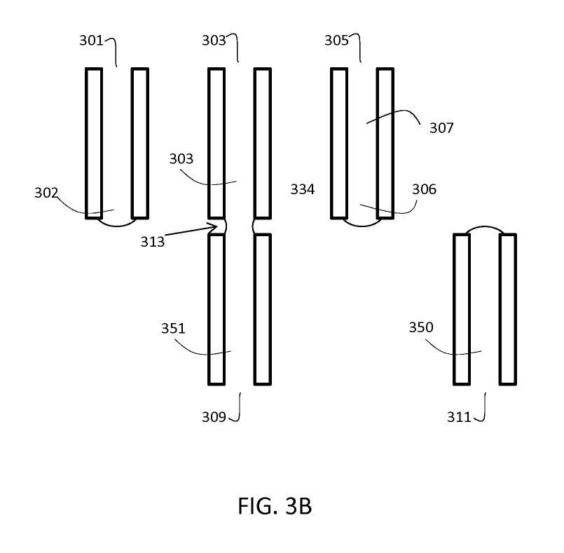

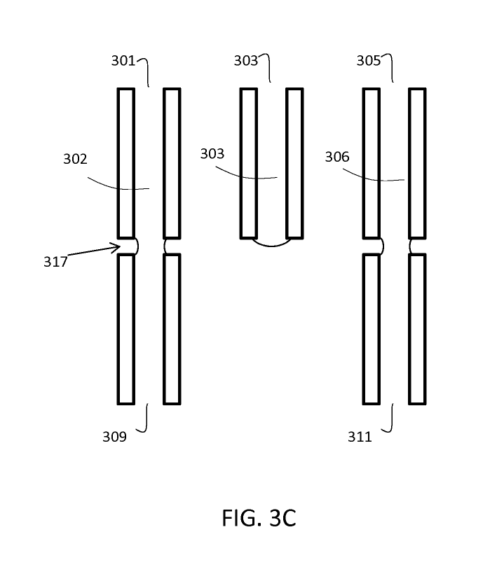

In certain embodiments, the systems of the invention are multi-channel systems. There is no limit to the number of channels that can be included in systems of the invention, nor is there any limitation on the configuration of the channels. A multi-channel system is described in FIG. 3A, and the skilled artisan will appreciate that this is only a single example of a multi-channel system. Numerous other multichannel systems and configurations can be envisioned by the skilled artisan and are within the scope of the invention.

FIGS. 3A-3C depict a multi-channel system configured to allow microfluidic channels to slide or move relative to each other to alter alignment of the channels. FIG. 3A shows multiple microfluidic channels 301, 303, and 305 which are open at ends 330, 332, and 334. Microfluidic channels 301, 303, and 305 each may contain a fluid, for example microfluidic channel 301 contains fluid 302. Fluids 302, 303, and 306 may be the same or different. Each fluid 302, 303, and 306 is retained in the microfluidic channels due to channel geometry and by forces such as surface tension. As discussed above, an aspect of the invention is that fluid does not flow from the microfluidic channel unless aligned with another microfluidic channel. Additionally, microfluidic channels 301, 303, and 305 may be slidable or moveable together or independent of one another. FIG. 3A also depicts microfluidic channels 309 and 311 which are open ended at 340 and 341. Microfluidic channels 309 and 311 are shown in FIG. 3A to contain fluids 350 and 351. However, microfluidic channels 309 and 311 are not required to contain fluids and may not contain fluids. Microfluidic channels 309 and 311 may be moved independent of one another or may be moved together. As shown in FIG. 3A, microfluidic channels 309 and 311 are positioned to be disengaged from microfluidic channels 301, 303, and 305. In this positioning of the microfluidic channels, microfluidic channels 301, 303, and 305 are prevented from flowing fluid due to the physical properties of the microfluidic channel and the fluid, e.g. surface tension.

Microfluidic channels 309 and 311 may be slid or moved to align with any of the microfluidic channels 301, 303, or 305. FIG. 3B depicts microfluidic channels 309 and 311 that has been moved or slid relative to microfluidic channels 301, 303, or 305. As shown in FIG. 3B, microfluidic channel 309 has been slid to engage at least one of microfluidic channels 301, 303, and 305. Moving or sliding of microfluidic channel 309 or 311 may involve movement in any plane or direction. In FIG. 3B, microfluidic channel 303 is aligned with microfluidic channel 309. The alignment may cause an air gap 313. Also, as discussed above, it is not necessary for the microfluidic channels to be aligned so that the microfluidic channels are flush. Rather, an air gap 313 may be present between the two microfluidic channels. The arrangement of microfluidic channels 303 and 309 is such when the channels are aligned, fluid bridges the air gap 313 and flows from microfluidic channel 303 into microfluidic channel 309. In this positioning, microfluidic channel 309 receives fluid 303 from microfluidic channel 303.

The microfluidic channels of the invention may be slid or move in several iterations. For example, as shown in FIG. 3C, microfluidic channel 309 has been slid to align with microfluidic channel 301. As discussed previously, microfluidic channel 309 was aligned with microfluidic channel 303 and received fluid 303. Microfluidic channel 309 now contains fluid 303 and fluid 302. In this embodiment, fluids are mixed from two different microfluidic channels. Additionally, as shown in FIG. 3C, microfluidic channel 311 is aligned with microfluidic channel 305. It should be appreciated that microfluidic channels 309 and 311 could be slid or moved at the same time, or independently of each other, depending on the configuration of microfluidic channels 309 and 311 and their respective substrates.

It should be appreciated that the multichannel systems of the invention may include numerous channels aligned in various planes of space. For example, FIGS. 3A-3C serve to illustrate how two levels of microfluidic channels can align to direct the flow of droplets within a microfluidic system. It should be appreciated that numerous levels of microfluidic channels may include a multichannel system, as discussed below.

### Fluids and Droplets

As discussed above, the microfluidic system of the present invention is capable of controlling the direction and flow of fluids and entities within the device. The term flow generally refers to any movement of liquid or solid through a device or in a method of the invention, and encompasses without limitation any fluid stream, and any material moving with, within or against the stream, whether or not the material is carried by the stream. The application of any force may be used to provide a flow, including without limitation, pressure, capillary action, electro-osmosis, electrophoresis, dielectrophoresis, optical tweezers, gravity, and combinations thereof, without regard for any particular theory or mechanism of action, so long as molecules, cells or virions are directed for detection, measurement or sorting according to the invention. Specific flow forces are described in further detail herein.

The flow stream in a microfluidic channel is typically, but not necessarily, continuous and may be stopped and started, reversed or changed in speed. As used herein, the term fluid stream or fluidic stream generally refers to the flow of a fluid, typically, generally in a specific direction. The fluidic stream may be continuous and/or discontinuous. A continuous fluidic stream is a fluidic stream that is produced as a single entity, e.g., if a continuous fluidic stream is produced from a channel, the fluidic stream, after production, appears to be contiguous with the channel outlet. The continuous fluidic stream is also referred to as a continuous phase fluid or carrier fluid. The continuous fluidic stream may be laminar, or turbulent in some cases.

Similarly, a discontinuous fluidic stream is a fluidic stream that is not produced as a single entity. The discontinuous fluidic stream is also referred to as the dispersed phase fluid or sample fluid. A discontinuous fluidic stream may have the appearance of individual droplets, optionally surrounded by a second fluid.

A droplet, as used herein, is an isolated portion of a first fluid that substantially or completely surrounded by a second fluid. In some cases, the droplets may be spherical or substantially spherical; however, in other cases, the droplets may be non-spherical, for example, the droplets may have the appearance of blobs or other irregular shapes, for instance, depending on the external environment. As used herein, a first entity is surrounded by a second entity if a closed loop can be drawn or idealized around the first entity through only the second entity. The dispersed phase fluid can include a biological/chemical material. The biological/chemical material can be tissues, cells, particles, proteins, antibodies, amino acids, nucleotides, small molecules, and pharmaceuticals. The biological/chemical material can include one or more labels known in the art. The label can be a DNA tag, dyes or quantum dot, or combinations thereof.

The term emulsion generally refers to a preparation of one liquid distributed in small globules (also referred to herein as drops or droplets) in the body of a second liquid. The first and second fluids are immiscible with each other. For example, the discontinuous phase can be an aqueous solution and the continuous phase can be a hydrophobic fluid such as an oil. This is termed a water in oil emulsion. Alternatively, the emulsion may be an oil in water emulsion. In that example, the first liquid, which is dispersed in globules, is referred to as the discontinuous phase, whereas the second liquid is referred to as the continuous phase or the dispersion medium. The continuous phase can be an aqueous solution and the discontinuous phase is a hydrophobic fluid, such as an oil (e.g., decane, tetradecane, or hexadecane). The droplets or globules of oil in an oil in water emulsion are also referred to herein as “micelles”, whereas globules of water in a water in oil emulsion may be referred to as “reverse micelles”.

The fluidic droplets may each be substantially the same shape and/or size. The shape and/or size can be determined, for example, by measuring the average diameter or other characteristic dimension of the droplets. The average diameter of a plurality or series of droplets is the arithmetic average of the average diameters of each of the droplets. Those of ordinary skill in the art will be able to determine the average diameter (or other characteristic dimension) of a plurality or series of droplets, for example, using laser light scattering, microscopic examination, or other known techniques. The diameter of a droplet, in a non-spherical droplet, is the mathematically-defined average diameter of the droplet, integrated across the entire surface. The average diameter of a droplet (and/or of a plurality or series of droplets) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain cases.

The droplet forming liquid is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible, with the population of molecules, cells or particles to be analyzed and/or sorted can be used. The fluid passing through the main channel and in which the droplets are formed is one that is immiscible with the droplet forming fluid. The fluid passing through the main channel can be a non-polar solvent, decane (e g., tetradecane or hexadecane), fluorocarbon oil, silicone oil or another oil (for example, mineral oil).

The dispersed phase fluid may also contain biological/chemical material (e.g., molecules, cells, or other particles) for combination, analysis and/or sorting in the device. The droplets of the dispersed phase fluid can contain more than one particle or can contain no more than one particle. For example, where the biological material comprises cells, each droplet preferably contains, on average, no more than one cell. However, in some embodiments, each droplet may contain, on average, at least 1000 cells. The droplets can be detected and/or sorted according to their contents.

The concentration (i.e., number) of molecules, cells or particles in a droplet can influence sorting efficiently and therefore is preferably optimized. In particular, the sample concentration should be dilute enough that most of the droplets contain no more than a single molecule, cell or particle, with only a small statistical chance that a droplet will contain two or more molecules, cells or particles. This is to ensure that for the large majority of measurements, the level of reporter measured in each droplet as it passes through the detection module corresponds to a single molecule, cell or particle and not to two or more molecules, cells or particles.

The parameters which govern this relationship are the volume of the droplets and the concentration of molecules, cells or particles in the sample solution. The probability that a droplet will contain two or more molecules, cells or particles (P≦₂) can be expressed as

P≦₂=1−{1+[cell]×V}×e^{-[cell]×V}

where “[cell]” is the concentration of molecules, cells or particles in units of number of molecules, cells or particles per cubic micron (μm³), and V is the volume of the droplet in units of μm³.

It will be appreciated that P≦₂ can be minimized by decreasing the concentration of molecules, cells or particles in the sample solution. However, decreasing the concentration of molecules, cells or particles in the sample solution also results in an increased volume of solution processed through the device and can result in longer run times. Accordingly, it is desirable to minimize to presence of multiple molecules, cells or particles in the droplets (thereby increasing the accuracy of the sorting) and to reduce the volume of sample, thereby permitting a sorted sample in a reasonable time in a reasonable volume containing an acceptable concentration of molecules, cells or particles.

The maximum tolerable P≦₂ depends on the desired purity of the sorted sample. The purity in this case refers to the fraction of sorted molecules, cells or particles that possess a desired characteristic (e.g., display a particular antigen, are in a specified size range or are a particular type of molecule, cell or particle). The purity of the sorted sample is inversely proportional to P≦₂. For example, in applications where high purity is not needed or desired a relatively high P≦₂ (e.g., P≦₂=0.2) may be acceptable. For most applications, maintaining P≦₂ at or below about 0.1, preferably at or below about 0.01, provides satisfactory results.

The fluidic droplets may contain additional entities, for example, other chemical, biochemical, or biological entities (e.g., dissolved or suspended in the fluid), cells, particles, gases, molecules, or the like. In some cases, the droplets may each be substantially the same shape or size, as discussed above. In certain instances, the invention provides for the production of droplets consisting essentially of a substantially uniform number of entities of a species therein (i.e., molecules, cells, particles, etc.). For example, about 90%, about 93%, about 95%, about 97%, about 98%, or about 99%, or more of a plurality or series of droplets may each contain the same number of entities of a particular species. For instance, a substantial number of fluidic droplets produced, e.g., as described above, may each contain 1 entity, 2 entities, 3 entities, 4 entities, 5 entities, 7 entities, 10 entities, 15 entities, 20 entities, 25 entities, 30 entities, 40 entities, 50 entities, 60 entities, 70 entities, 80 entities, 90 entities, 100 entities, etc., where the entities are molecules or macromolecules, cells, particles, etc. In some cases, the droplets may each independently contain a range of entities, for example, less than 20 entities, less than 15 entities, less than 10 entities, less than 7 entities, less than 5 entities, or less than 3 entities in some cases. In some embodiments, a droplet may contain 100,000,000 entities. In other embodiments, a droplet may contain 1,000,000 entities.

In a liquid containing droplets of fluid, some of which contain a species of interest and some of which do not contain the species of interest, the droplets of fluid may be screened or sorted for those droplets of fluid containing the species as further described below (e.g., using fluorescence or other techniques such as those described above), and in some cases, the droplets may be screened or sorted for those droplets of fluid containing a particular number or range of entities of the species of interest, e.g., as previously described. Thus, in some cases, a plurality or series of fluidic droplets, some of which contain the species and some of which do not, may be enriched (or depleted) in the ratio of droplets that do contain the species, for example, by a factor of at least about 2, at least about 3, at least about 5, at least about 10, at least about 15, at least about 20, at least about 50, at least about 100, at least about 125, at least about 150, at least about 200, at least about 250, at least about 500, at least about 750, at least about 1000, at least about 2000, or at least about 5000 or more in some cases. In other cases, the enrichment (or depletion) may be in a ratio of at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵, or more. For example, a fluidic droplet containing a particular species may be selected from a library of fluidic droplets containing various species, where the library may have about 100, about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, or more items, for example, a DNA library, an RNA library, a protein library, a combinatorial chemistry library, etc. In certain embodiments, the droplets carrying the species may then be fused, reacted, or otherwise used or processed, etc., as further described below, for example, to initiate or determine a reaction.

In some aspects of the invention the droplets may comprise sample fluid, discussed below. It should be appreciated that the sample fluid varies depending on the biological or chemical assay being performed within the droplet. In assays involving biological processes, the sample fluid is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. In assays involving amplification and detection of nucleic acids, any liquid or buffer that is physiologically compatible with nucleic acid molecules can be used.

In assays related to cell culturing, the sample fluid may comprise cell medium. The cell medium provides the necessary nutrients, growth factors, and hormones for cell growths, as well as regulating the pH and the osmotic pressure of the culture. The cell culture medium may allow for and support growth of the cells thus being cultured, or the cell medium is a maintenance medium. Growth is understood as an increase in viable cell density during at least a certain period of the cell culture. A maintenance medium is a cell culture medium which supports cell viability but which does not encourage cell growth. See for example, cell culture medium related patents: U.S. Pat. No. 4,038,139, 1977; U.S. Pat. No. 7,258,998, 2007; U.S. patent application Ser. No. 13/497,707, 2010; U.S. Pat. No. 8,338,177, 2012; and U.S. patent application Ser. No. 13/695,002, 2011. The growth medium controls the pH of the culture and buffers the cells in culture against fluctuations in the pH. This buffering may be achieved by including an organic (e.g., HEPES) or CO₂ bicarbonate based buffer. Control of pH is needed to ensure the growth and health of cells in culture. Most normal mammalian cell lines grow well at pH 7.4, and there is very little variability among different cell strains.

The carrier fluid is one that is immiscible with the sample fluid. As used herein, carrier fluid and immisible fluid may be used interchangeably. The carrier fluid can be a non-polar solvent, decane (e g., tetradecane or hexadecane), fluorocarbon oil, silicone oil or another oil (for example, mineral oil). In an aspect of the invention, the carrier fluid forms a meniscus at the open end of the microfluidic channel, caused by surface tension. The meniscus can be either convex or concave, depending on the carrier fluid and the surface of the microfluidic channel.

In certain embodiments, the carrier fluid contains one or more additives, such as agents which increase, reduce, or otherwise create non-Newtonian surface tensions (surfactants) and/or stabilize droplets against spontaneous coalescence on contact. Surfactants can include Tween, Span, fluorosurfactants, and other agents that are soluble in oil relative to water. In some applications, performance is improved by adding a second surfactant, or other agent, such as a polymer or other additive, to the sample fluid. Surfactants can aid in controlling or optimizing droplet size, flow and uniformity. Furthermore, the surfactant can serve to stabilize aqueous emulsions in fluorinated oils from coalescing.

In certain embodiments, the droplets may be coated with a surfactant or a mixture of surfactants. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglycerl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

Droplet Formation by Fluid Segmentation within Movable Channels

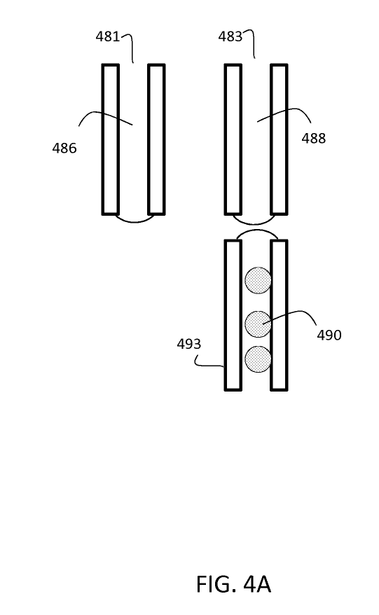

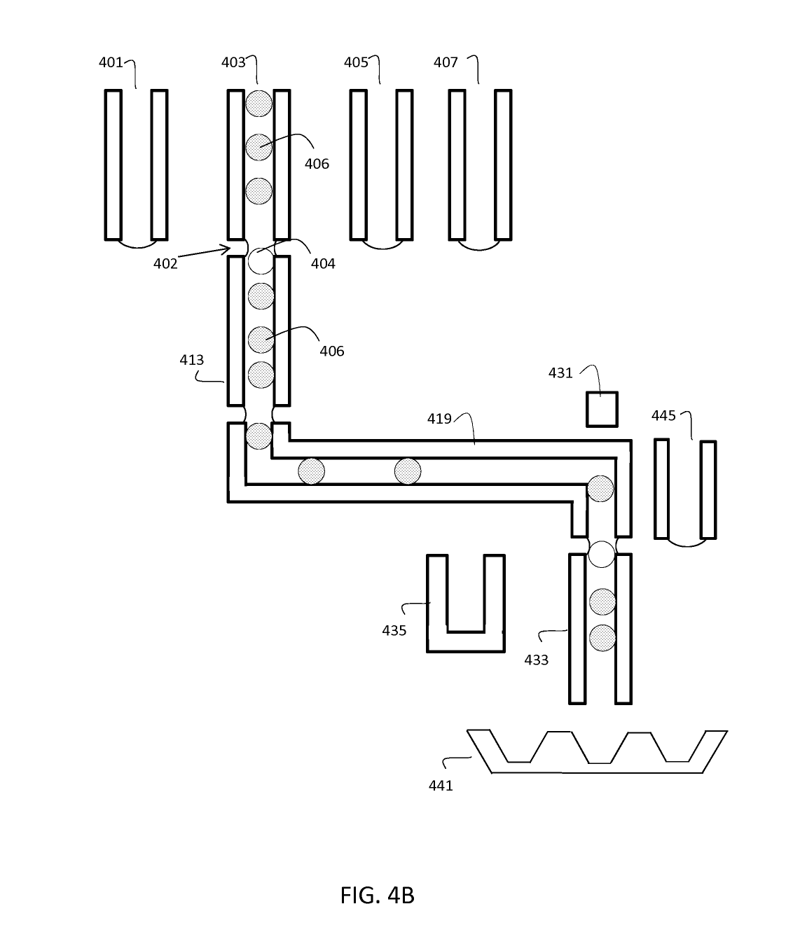

Microfluidic systems of the invention can be used to form droplets through the movement of the different channels. The microfluidic device can be used to produce single or multiple emulsions with precise control of both the contents and size of the drops. FIG. 4A depicts an arrangement of microfluidic channels for forming droplets. Microfluidic channel 481 contains a fluid 486 which is immiscible with aqueous fluids, such as an oil. Microfluidic channel 483 contains an aqueous fluid 488. To create droplets within microfluidic channel 493, microfluidic channels aligns with microfluidic channel 481 to form an air gap (not shown) where the fluid 486 spans the air gap and flows into microfluidic channel 493. As discussed herein, microfluidic channels are aligned by sliding one microfluidic channel proximate to another microfluidic channel, causing an air gap between the microfluidic channels to form. Microfluidic channel 493 is disengaged or misaligned with microfluidic channel 481 to allow a small volume of fluid 486 to flow into microfluidic channel 493. Microfluidic channel 493 is then aligned with microfluidic channel 483 to allow a small volume of aqueous fluid 488 to flow into microfluidic channel 493. Fluid 488 is immiscible with fluid 486 present in microfluidic channel 493 causing droplets 490 to form, which contains fluid 488. Microfluidic channel 493 can align in an alternating pattern to form droplets 490. In the alternative, oil droplets can be formed in an aqueous phase using the technique described above.

FIG. 4B depicts an arrangement using microfluidic systems of the invention to form droplets from the fluid contained in microfluidic channel 401 and insert the formed droplets into a stream of droplets from microfluidic channel 403. As shown in FIG. 4B, microfluidic channel 413 can align with several microfluidic channels: 401, 403, 405, and 407. In aspect of the invention, microfluidic channel 413 can be slid or moved in order to align with microfluidic channels 401, 403, 405, and 407. Microfluidic channels 401, 403, 405, and 407 may be stationary or moveable. Furthermore, microfluidic channels 401, 403, 405, and 407 may be moved together, or may be moved independently, depending on the arrangement of substrates employed. Microfluidic channel 401 contains a fluid. As shown in FIG. 4B, microfluidic channel 403 contains droplets 406 within an immiscible fluid.

As shown in FIG. 4B, microfluidic channel 413 contains droplets 406 and 404. Droplet 404 contains the fluid from microfluidic channel 401, and the fluid in microfluidic channel 401 is immiscible with the fluid in microfluidic channel 413. Microfluidic channel 413 was aligned with microfluidic channel 401 for a span of time to only allow for a small volume of fluid to pass from microfluidic channel 401 into microfluidic channel 413. This small volume of fluid formed into a droplet 404 within microfluidic channel 413, which contains a fluid immiscible with the droplets in microfluidic channel 403 and the fluid in microfluidic channel 401. Alignment may occur to allow for a small volume of fluid to pass between microfluidic channels to generate a droplet in the receiving microfluidic channel. As shown in FIG. 4B, microfluidic channel 413 is aligned with microfluidic channel 403 so that a portion of fluid spans an air gap 402 to thereby flow fluid and droplets 406 from microfluidic channel 403 into microfluidic channel 413. Microfluidic channel 413 may slide or move to align with microfluidic channels 401, 403, 405, and 407. Microfluidic channel 413 can be slid to align with any of the microfluidic channels, or microfluidic channel 413 can be positioned so as not to align with any microfluidic channel, and therefore does not receive fluid or droplets. Microfluidic channel 413 may align with microfluidic channel 419. Microfluidic channel 419 may be movable or may be stationary. Alignment between microfluidic channel 413 and microfluidic channel 419 allows for flow between the two channels.

As shown in FIG. 4B, microfluidic channel 433 is aligned with microfluidic channel 419 to allow fluid to flow between the two microfluidic channels. Microfluidic channel 433 can be slid to disengage from microfluidic channel 419. A chamber 435 can be slid to engage with microfluidic channel 419 to thereby allow fluid to flow from microfluidic channel 419 into chamber 435. Chamber 435 can be a waste chamber. As shown in FIG. 4B, microfluidic channel 433 can align with wells in a well plate 441 to deliver fluid from microfluidic channel 433 to the wells in the well plate 441.

### Droplet Formation by Separate Droplet Generation Module

In other embodiments, droplets are formed using a separate droplet generation module. The droplet generation module can be fluidically coupled to the system so that generated droplets can directly flow into different channels of the system. Alternatively, the droplet generation module can be a separate component in which droplets are generated and collected in a vessel and then separately loaded into systems of the invention. Droplet generation may be accomplished by numerous techniques. The droplets may be formed, for example, by dipping an open ended tube into a vessel. Exemplary sample acquisition devices are shown in McGuire et al. (U.S. patent application publication No. 2010/0294048), the content of which is incorporated by reference herein in its entirety. Parameters such as channel diameter, dipping time, and system flow, may be adjusted so that wrapped droplets are formed of different volumes. In certain embodiments, a droplet contains no more than a single entitle, such as a single biological molecule or a single cell.

Methods of the invention involve forming sample droplets in which some droplets contain zero, one, or multiple entities, such as cells. In the preferred embodiment, the distribution of entities (e.g., cells) within droplets obeys the Poisson distribution. However, methods for non-Poisson loading of droplets are known to those familiar with the art, and include but are not limited to active sorting of droplets, such as by laser-induced fluorescence, or by passive one-to-one loading. The description that follows assumes Poisson loading of droplets, but such description is not intended to exclude non-Poisson loading.

In certain embodiments, the droplets are aqueous droplets that are surrounded by an immiscible carrier fluid. In other embodiments, the droplets are non-aqueous droplets surrounded by an immiscible fluid, such as oil droplets in a water continuous phase. Methods of forming such droplets are shown for example in Link et al. (U.S. patent application Nos. 2008/0014589, 2008/0003142, and 2010/0137163), Stone et al. (U.S. Pat. No. 7,708,949 and U.S. patent application No. 2010/0172803), and Anderson et al. (U.S. Pat. No. 7,041,481 and which reissued as RE41, 780), he content of each of which is incorporated by reference herein in its entirety.

In some embodiments, the sample fluid is aqueous, such as when employing a culture medium. The sample fluid is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with the sample can be used. In the preferred embodiment, the sample fluid comprises cell medium, as disclosed above. The sample fluid comprises the necessary components to ensure cell health and growth. As discussed above, any laboratory produced or commercially available cell medium may be employed.

As discussed above, the carrier fluid is one that is immiscible with the sample fluid. The carrier fluid can be a non-polar solvent, decane (e g., tetradecane or hexadecane), fluorocarbon oil, silicone oil or another oil (for example, mineral oil). In a preferred embodiment of the invention, the carrier fluid has a high surface tension and therefore, is retained a microfluidic channel at an open end. In an aspect of the invention, the carrier fluid forms a meniscus at the open end of the microfluidic channel, caused by surface tension. The meniscus can be either convex or concave, depending on the carrier fluid and the surface of the microfluidic channel.

Key elements for using microfluidic channels to process droplets include producing droplet of the correct volume, producing droplets at the correct frequency and bringing together a first stream of sample droplets with a second stream of sample droplets in such a way that the frequency of the first stream of sample droplets matches the frequency of the second stream of sample droplets. In some embodiments of the present invention, gravitational forces control the flow rate within the device.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. Feedback on the infusion rates of the carrier fluid and the dispersed fluid provides droplets that are uniform in size and generated at a fixed frequency over arbitrarily long periods of time.

Aspects of the invention may employ the use of a microfluidic droplet generator device. The droplet generator device is configured to be in fluid communication with the multichannel system of the invention. For example, see US Patent Application 20140017150, which is incorporated by reference. The generator device comprises a substrate; a microfluidic channel formed in the substrate; a fluid outlet in fluid communication with the microfluidic channel; and a mechanical element configured such that vibration of the mechanical element causes droplet dispensing from the fluid outlet. It should be appreciated by one of skill in the art that droplets can be generated by alternative methods and techniques.

### Droplet Pick-up

Droplets of the invention may be picked-up or transferred from a vessel to a microfluidic channel. A vessel may include any structure that contains fluids or droplets, for example, wells within a well plate. Without being bound by theory, if a microfluidic channel is sufficiently narrow and the liquid adhesion to the microfluidic channel wall is sufficiently strong, surface tension can draw liquid up the microfluidic channel in a phenomenon known as capillary action. The height to which the column is lifted is given by:

$$h = \frac{2 {ϒ}_{la} cos Θ}{pgr}$$

where h is the height the liquid is lifted, Yₗₐ is the liquid-air surface tension, p is the density of the liquid, r is the radius of the capillary, g is the acceleration due to gravity, Θ is the angle of contact described above. In an aspect of the invention, microfluidic channels can be positioned at or near the surface of a liquid and draw the liquid up into the microfluidic channel.

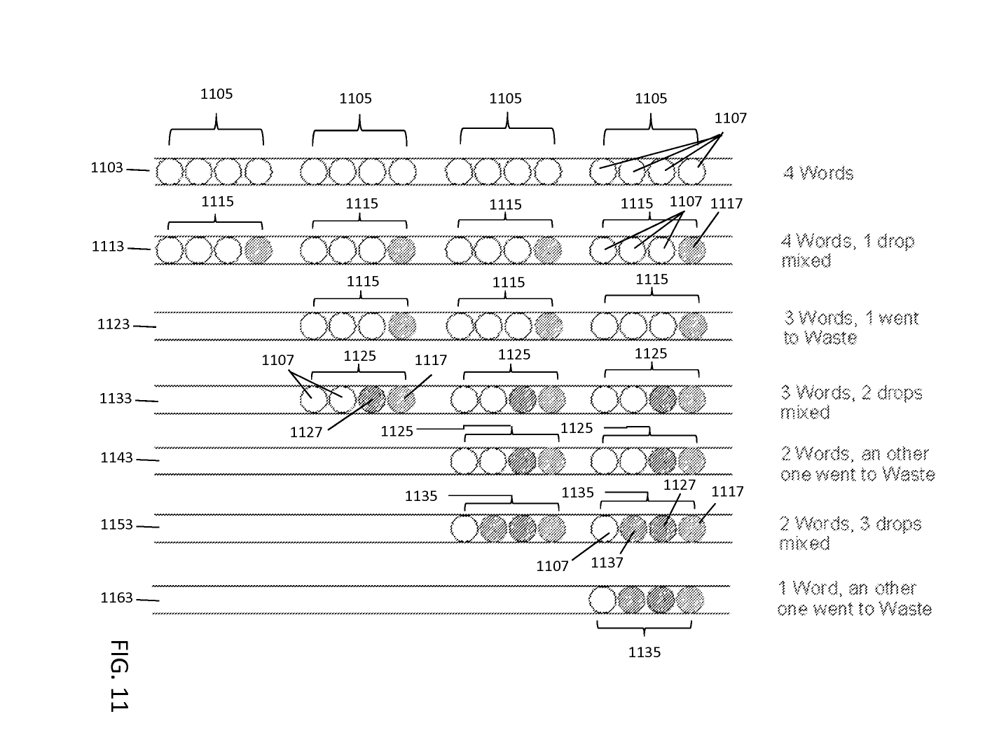

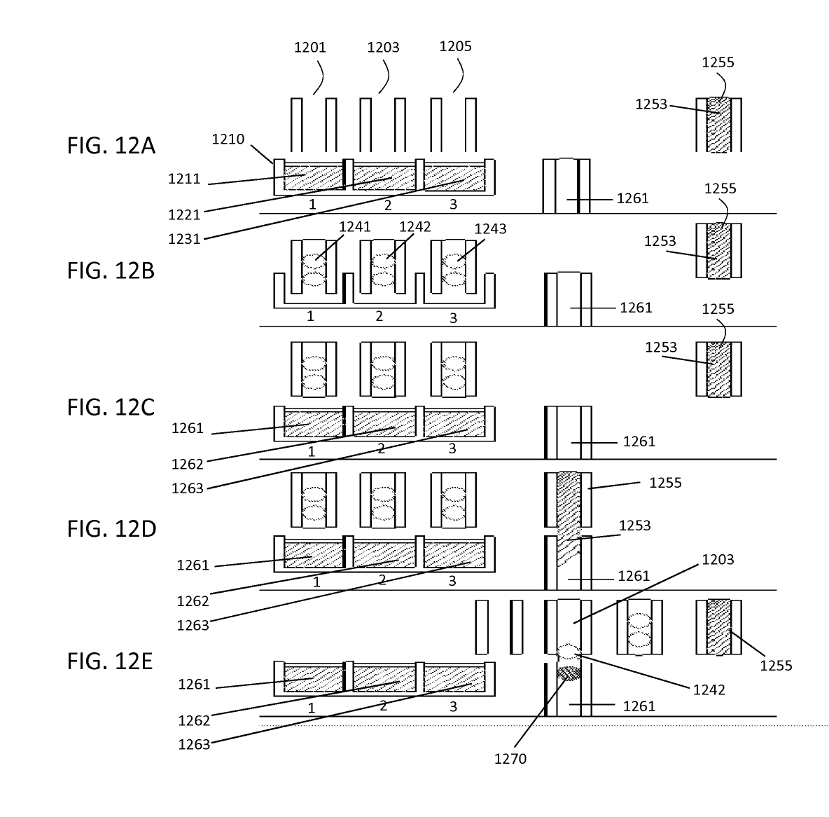

As shown in FIG. 12A, microfluidic channels 1201, 1203, and 1205 are positioned proximate to well plate 1210. Well Plate 1210 contains wells 1, 2, and 3. Fluids 1211, 1221, and 1231 are contained within wells 1, 2, and 3 of well plate 1210. Fluids 1211, 1221, and 1231 may contain droplets. As shown in FIG. 12B, microfluidic channels 1201, 1203, and 1205 are placed at or near the surface of fluids 1211, 1221, and 1231 and draws up fluids 1211, 1221, and 1231 into microfluidic channels 1201, 1203, and 1205. Wells 1, 2, and 3 are emptied of fluid. Fluids 1211, 1221, and 1231 may contain droplets 1241, 1242, and 1243, that are also drawn up into microfluidic channels 1201, 1203, and 1205 or the up-down movement of channels 1201, 1203, and 1205 in fluids 1211, 1221, and 1231 may result in formation of droplets 1241, 1242, and 1243 within channels 1201, 1203, and 1205.

In FIG. 12C, the microfluidic channels 1201, 1203, and 1205 are positioned proximate to wells 1, 2, and 3. Wells 1, 2, and 3 are replenished with fluids 1261, 1262, and 1263. Fluids 1261, 1262, and 1263 may be the same as fluids 1211, 1221, and 1231, or fluids 1261, 1262, and 1263 may be different. Similar to FIG. 12B, microfluidic channels 1201, 1203, and 1205 are placed at or near the surface of fluids 1261, 1262, and 1263and draws up fluids 1261, 1262, and 1263 into microfluidic channels 1201, 1203, and 1205. Wells 1, 2, and 3 are emptied of fluid. Fluids 1261, 1262, and 1263 may contain droplets that are also drawn up into microfluidic channels 1201, 1203, and 1205 or the up-down movement of channels 1201, 1203, and 1205 in fluids 1261, 1262, and 1263 may result in formation of droplets within channels 1201, 1203, and 1205.

FIG. 12D shows the alignment of microfluidic channel 1261 with microfluidic channel 1255. Microfluidic channel 1255 contains fluid 1253. Aligning microfluidic channel 1261 and microfluidic channel 1255 causes fluid 1253 to flow from microfluidic channel 1255 into microfluidic channel 1261. The volume of fluid 1253 can be of any size; whereas a small volume causes a droplet to form. As shown in FIG. 12E, after fluid 1253 has flowed into microfluidic channel 1261, microfluidic channel 1203 aligns with microfluidic channel 1261 to allow for droplets 1242 to flow into microfluidic channel 1261. The fluid 1255 may form a droplet and, using techniques disclosed herein, merges with droplet 1242 to form droplet 1270 (discussed below).

### Droplets and the Microfluidic System

The microfluidic device of the invention may be utilized to flow droplets within microfluidic channels. As discussed in detail above in regards to FIGS. 3A-3C, the microfluidic channels of the present invention can be aligned to allow the flow of fluids therebetween. In some embodiments of the present invention, the microfluidic system may be utilized to flow droplets within and between microfluidic channels. The droplets will typically be flowing in a carrier fluid, such as an oil.

In certain embodiments of the present invention, the fluid within the microfluidic channel contains droplets. As discussed above, droplets may be composed or various fluids and various components. As discussed previously, the microfluidic channels of the invention can be slid to align in order to select the path of the droplets within the microfluidic system. As the droplets flow through the channels, flow can be stopped within a microfluidic channel by misalignment with another microfluidic channel. Flow may resume once the microfluidic channel is aligned with another microfluidic channel such that flow occurs therebetween.

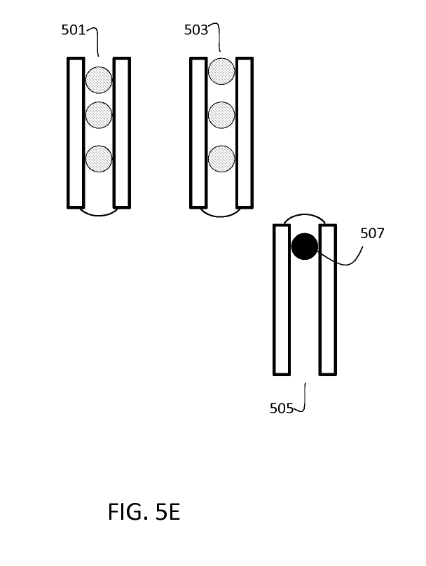

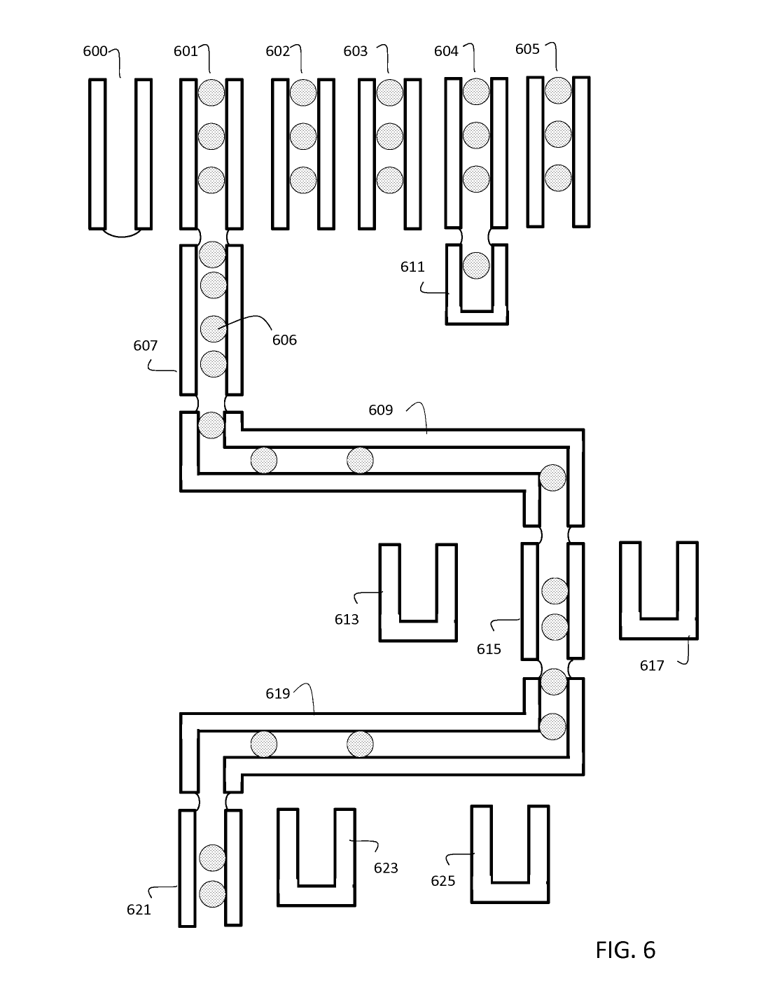

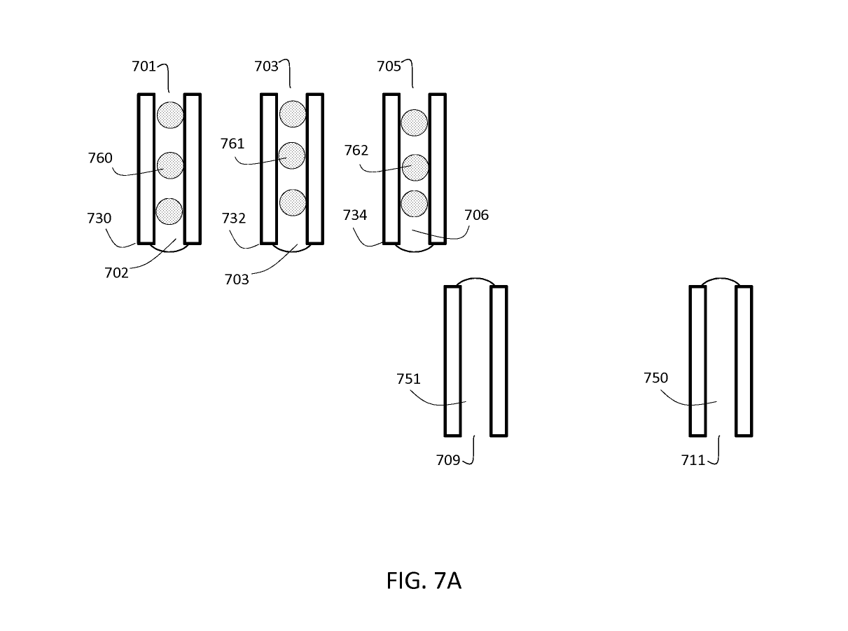

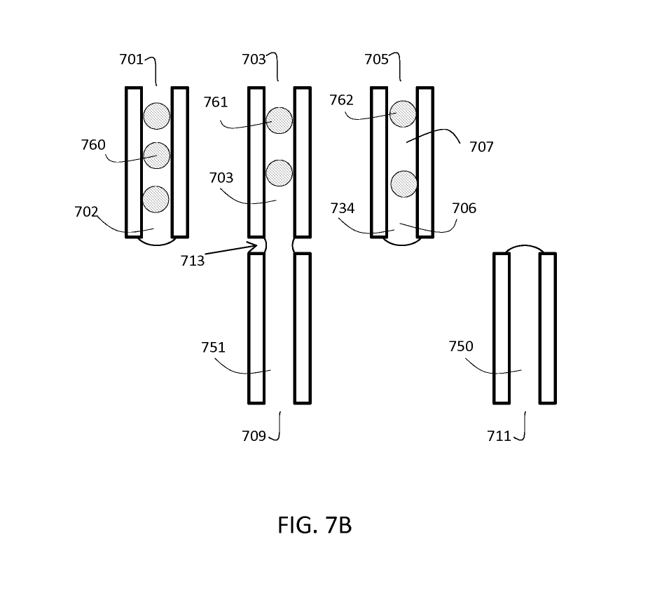

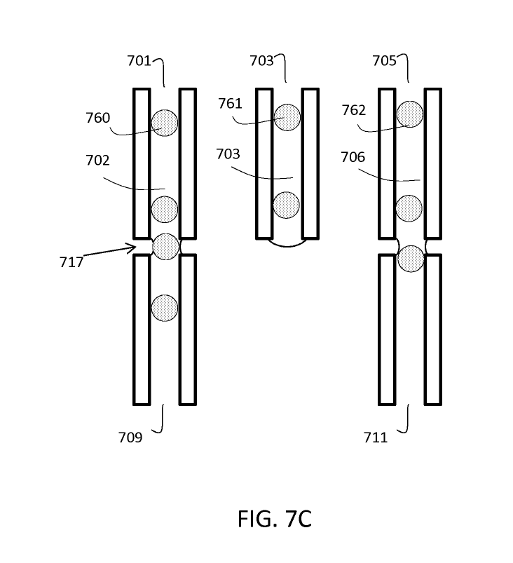

FIGS. 7A-7C depict a multi-channel system configured to allow microfluidic channels to slide or move relative to each other to alter alignment of the channels. FIG. 7A shows multiple microfluidic channels 701, 703, and 705 which are open at ends 730, 732, and 734. Microfluidic channels 701, 703, and 705 each may contain a fluid; for example microfluidic channel 701 contains fluid 702. Fluids 702, 703, and 706 may be composed of the same components or may be composed of differing components. Each fluid 702, 703, and 706 is retained in the microfluidic channel by forces such as surface tension. As discussed above, an aspect of the invention is that fluid does not flow from the microfluidic channel unless aligned with another microfluidic channel. Also, each microfluidic channel 701, 703, and 705 contains droplets 760, 761, and 762. As discussed above, droplets may be composed of various fluids and components. Additionally, the droplets 701, 703, and 705 may comprise the same materials and components, or they droplets may comprise differing materials and components. Additionally, microfluidic channels 701, 703, and 705 may be slidable or moveable together or independent of one another. FIG. 7A also depicts microfluidic channels 709 and 711 which are open ended at 740 and 741. It should be appreciated that microfluidic channels 709 and 711 are shown in FIG. 7A to contain fluids 750 and 751. However, it should also be appreciated that microfluidic channels 709 and 711 may not contain fluids. Microfluidic channels 709 and 711 may be moved independent of one another or may be moved together. It is an aspect of the invention that microfluidic channels may be arranged in any configuration and manner. As shown in FIG. 7A, microfluidic channels 709 and 711 are positioned to be disengaged from microfluidic channels 701, 703, and 705. In this positioning of the microfluidic channels, microfluidic channels 701, 703, and 705 are prevented from flowing fluid due to the physical properties of the microfluidic channel and the immiscible fluid, e.g. surface tension, as discussed above.

Microfluidic channels 709 and 711 may be slid or moved to align with any of the microfluidic channels 701, 703, or 705. FIG. 7B depicts microfluidic channels 709 and 711 that has been moved or slid relative to microfluidic channels 701, 703, or 705. As shown in FIG. 7B, microfluidic channel 709 has been slid to engage at least one of microfluidic channels 701, 703, and 705. It should be appreciated that the moving or sliding of microfluidic channel 709 or 711 may involve movement in any plane or direction. In FIG. 7B, microfluidic channel 703 is aligned with microfluidic channel 709. The alignment may cause an air gap 713. Also, as discussed above, it is not necessary for the microfluidic channels to be aligned so that the microfluidic channels are flush. Rather, an air gap 713 may be present between the two microfluidic channels. The alignment of microfluidic channels 703 and 709 forms an air gap at 713. The formation of the air gap 713 results in a portion of fluid spanning air gap 713 and allows for fluid 703 and droplets 761 to flow from microfluidic channel 703 into microfluidic channel 709. In this positioning, microfluidic channel 709 receives fluid 703 and droplets 761 from microfluidic channel 703.

The microfluidic channels of the invention may be slid or move in several iterations. For example, as shown in FIG. 7C, microfluidic channel 709 has been slid to align with microfluidic channel 701. As discussed previously, microfluidic channel 709 was aligned with microfluidic channel 703 and received fluid 703 and droplets 761. Microfluidic channel 709 now contains fluid 703 and fluid 702 and droplets 761 and 760. In this embodiment, the components of the two different microfluidic channels are mixed. Additionally, as shown in FIG. 7C, microfluidic channel 711 is aligned with microfluidic channel 305. Microfluidic channels 709 and 711 can be slid or moved at the same time, or independently of each other, depending on the configuration of microfluidic channels 709 and 711 and their respective substrates.

Multichannel systems of the invention may include numerous channels aligned in various planes of space. For example, FIGS. 7A-7C serve to illustrate how two levels of microfluidic channels can align to direct the flow of droplets within a microfluidic system. It should be appreciated that different system architectures within the scope of the invention.

### Droplet Merging

Systems of the invention can also be used for droplet merging or coalescing. The fluidic droplets may be of unequal size in certain cases. In certain cases, one or more series of droplets may each consist essentially of a substantially uniform number of entities of a species therein (i.e., molecules, cells, particles, etc.). The fluidic droplets may be coalesced to start a reaction, and/or to stop a reaction, in some cases. For instance, a reaction may be initiated when a species in a first droplet contacts a species in a second droplet after the droplets coalesce, or a first droplet may contain an ongoing reaction and a second droplet may contain a species that inhibits the reaction. Embodiments of the invention are directed to alignment of microfluidic channels to promote the coalescence of fluidic droplets.

In embodiments in which droplets do not contain cells, droplets may coalesce, for example, upon application of an electric field. An aspect of the invention incorporates the application of an electric field. The applied electric field may induce a charge, or at least a partial charge, on a fluidic droplet surrounded by an immiscible fluid. Upon the application of an electric field, for example by producing a voltage across electrodes using a voltage source, droplets are induced to assume opposite charges or electric dipoles on the surfaces closest to each other, causing the droplets to coalesce.

In another aspect of the invention, droplets may fuse by joining the fluid between two droplets, which may occur due to the charge-charge interactions between the two fluids. The creation of the bridge of fluid between the two droplets thus allows the two droplets to exchange material and/or coalesce into one droplet. Thus, in some embodiments, the invention provides for the coalescence of two separate droplets into one coalesced droplet in systems where such coalescence ordinarily is unable to occur, e.g., due to size and/or surface tension, etc.

In droplets containing cells, use of electrodes or electric fields to merge droplets may need to be avoided. Coalescing of droplets without the application of an electric field is described by Xu et al., “Droplet Coalescence in Microfluidic Systems,” Micro and Nanosystems, 2011, 3, 131-136, which is incorporated by reference. The method described by Xu et al. merges droplets by allowing two droplets to have close contact with each other, causing the liquids of the two droplets to form a thin bridge between the two droplets. The methods described by Xu et al. are passive, where an external energy source is not needed. Passive merging can also be accomplished by channel geometry. See for example Gu et al., Int J Mol Sci. 2011; 12(4): 2572-2597, published online Apr. 15, 2011. Doi 10.3390/ijms12042572, which is incorporated by reference. For example, droplet merging consists of a widening channel follow by a narrower channel. In this geometry the droplet velocity decreases in the widening channel, after which it increases again upon entry in the narrow channel. Droplets close in proximity merge.

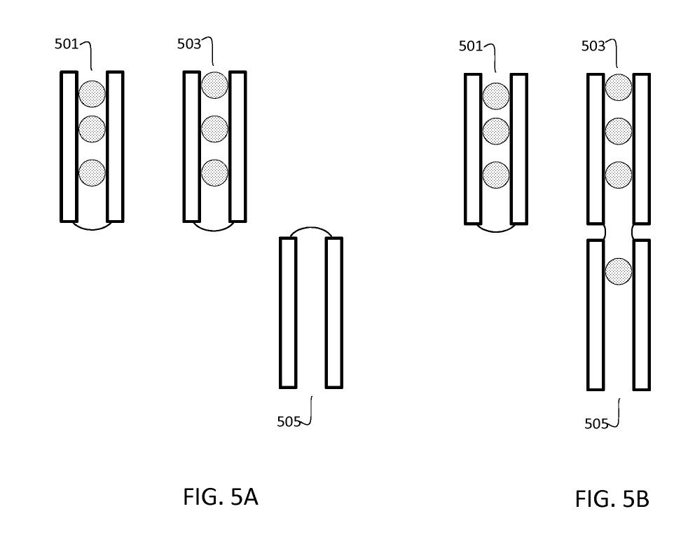

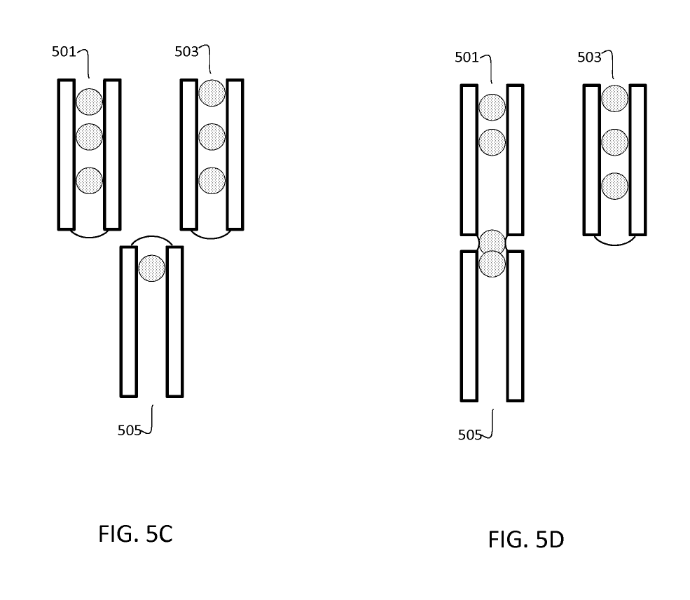

Another embodiment of the device is shown at FIG. 5A-5E. As shown in FIG. 5A, microfluidic channels 501 and 503 contain carrier fluid and at least one droplet. Microfluidic channel 505 is not aligned with either microfluidic channel 501 or 503. The carrier fluid and the droplets are not able to flow out of the open end of microfluidic channels 501 or 503. FIG. 5B shows microfluidic channel 503 aligned with microfluidic channel 505, thereby allowing flow of carrier fluid and droplets between microfluidic channel 503 and microfluidic channel 505. FIG. 5C shows an embodiment in which microfluidic channel 505 received a droplet when aligned to microfluidic channel 503, and the alignment is then disengaged. FIG. 5D shows microfluidic channel 505 aligned with microfluidic channel 501 to receive carrier fluid and a droplet from microfluidic channel 501. The droplets within microfluidic channel 505 can placed close together, causing passive merging. As shown in FIG. 5E, within microfluidic channel 505, the droplets are coalesced 507. As discussed above, coalesces may be accomplished by the application of an electric field. In droplets where cells are not present, electrodes may be located proximate to microfluidic channels to create an electric field to cause droplets to coalesce. In droplets in which cells are not present, passive merging can be accomplish by positioning droplets next to one another. In some embodiments in which droplets are present, aligning microfluidic channels to position droplets close together causes passive merging, as described above.

### Splitting of Droplets

The microfluidic device of the present invention may be used to split droplets, by known methods. Droplet splitting has numerous applications and is particularly useful for culturing cells in droplets, which is discussed in more detail below. Examples include the splitting of droplets by directing the droplets towards an obstacle, such as is disclosed in U.S. patent application Ser. No. 11/024,228, filed Dec. 28, 2004, entitled “Method and Apparatus for Fluid Dispersion,” by Stone, et al., now U.S. Pat. No. 7,708,949, issued May 4, 2010. Several methods for droplet splitting at a T-junction in a microfluidic system are known in the art. See for example, Link et al., (Phys. Rev. Lett. 2004, 92, 4.) and Nie et al. (Anal. Chem. 2010, 82, 7852-7856). With an appropriate geometry it is possible to split a droplet in a ratio that is inversely proportional to flow resistances in each of the two branches of a T-junction. Other disclosed methods of droplet splitting are shown for example in International patent application publication number WO 2013/014215, which is herein incorporated by reference.

As shown in FIG. 8, a microfluidic channel 804 is configured to have a branched portion 808. The microfluidic channel 804 contains droplets 806 within an immiscible fluid 802. An obstacle 810 is positioned so droplets 806 contact the obstacle and split into at least two different parts 820 and 821. It should be appreciated that the open ends of the microfluidic channel 831 and 830 are able to align with other microfluidic channels or chambers (discussed below).

### Detectors

An aspect of the invention incorporates a detector within the devices and methods of the invention. The detection apparatuses can be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module. Further description of detection modules and methods of detecting species in droplets are shown in Link et al. (U.S. patent application Nos. 2008/0014589, 2008/0003142, and 2010/0137163).

### Droplet Collection

The multichannel system of the present invention is able to direct and manipulate the flow of fluids and droplets contained within the microfluidic system. An aspect of the invention involves the directing of droplets to microfluidic channels, waste chambers, or collection chambers. In a preferred embodiment of the invention, after processing within the microfluidic device, the droplets are directed to a collection chamber for removal, further processing, or detection. It should be appreciated that in conjunction with slidable microfluidic channels, slidable and moveable chambers can be incorporated into the devices and methods of the present invention.

FIG. 6 depicts another embodiment of the invention. As shown in FIG. 6, a plurality of microfluidic channels, 600, 601, 602, 603, 604, and 605 contain carrier fluid and droplets. Microfluidic channel 601 is aligned with microfluidic channels 607 to allow fluid and droplets to flow from microfluidic channels 601 to microfluidic channels 607 and then to microfluidic channels 609. Microfluidic channel 603 is aligned with microfluidic channels 611, which can serve as a microfluidic channels and as a chamber. The chamber can a waste chamber, a collection chamber, or other purpose chamber employed with the device and methods of the invention. As shown in FIG. 6, microfluidic channels 611 accepts fluid from microfluidic channels 603, and microfluidic channels 611 serves as a waste chamber. Microfluidic channels 600, 601, 602, 603, 604, and 605 may be attached to or positioned within a surface, wherein the surface is movable. It should be appreciated that microfluidic channels 600, 601, 602, 603, 604, and 605 may be moved together or independently of one another. Microfluidic channel 607 also may be slid or remain in a stationary position. Microfluidic channel 611 may be slid or remain in a stationary position. Fluid and droplets flow into microfluidic channel 609 when a microfluidic channel aligns with microfluidic channel 609. Microfluidic channel 609 may be slidable or stationary. Fluid and droplets can flow from microfluidic channel 609 to microfluidic channel 619 when microfluidic channel 615 is aligned therebetween. Microfluidic channel 613 can align with microfluidic channel 609 to divert the fluid and/or droplets to a waste chamber. Microfluidic channel 617 can align with microfluidic channel 617 to diver the fluid and/or droplets to a collection chamber. Microfluidic channels 621, 623, and 625 can align with microfluidic channel 619 to divert or direct the flow of droplets into another microfluidic channel, a waste chamber or a collection chamber. Alternatively, droplets can be diverted into an incubation chamber for further processing.

### Circulating Channel

As discussed above, microfluidic channels may be nonlinear, or even contain branches. In some embodiments, microfluidic channels of the present invention may be substantially circular. In some embodiments of the invention, microfluidic channels of the present invention may be configured to provide a continuous circular path. It should be appreciated that the configurations of the present invention are non-limiting and various configurations and pathways can be employed in designing a multichannel system according to the invention.

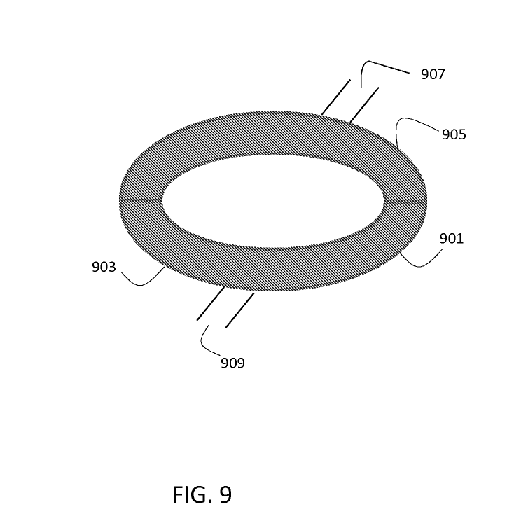

As shown in FIG. 9, a microfluidic channel is substantially circular 905. Within the circular microfluidic channel 905, fluids and droplets may be flowed, as discussed above. The microfluidic channel 905 is configured with a microfluidic channel 907. Microfluidic channel 907 may be used to introduce fluid and/or droplets into the circular microfluidic channel 905. The circular microfluidic channel 905 is configured with an outlet microfluidic channel 909. Microfluidic channels 907 and 909 may be aligned with other microfluidic channels or with chambers of a multichannel system. Circular microfluidic channel 905 may be configured with a rotor (not shown).

In some embodiment of the invention, the circulating channel is used so that cell culturing can be performed in systems of the invention. Cells are held in microfluidic aqueous droplets that are separated from one another by silicone oil, or any immiscible fluid. These droplets are then introduced into the circular microfluidic channel. The droplets are held in a circular cross-section channel which has a rotating inner wall and a stationary outer wall. Rotation is achieved by a rotor or similar device. In that manner, the droplets can circulate for as long as is required, for example for culturing of a cell in the droplet. Waste material from the cells diffuses to the lower portion of the circular path. An outlet channel configured at the lower portion of the circular path removes the waste material. An inlet channel configured to deliver fluids into the circular path allows for the introduction of additional fluids, such as cell medium.

In a preferred embodiment of the invention, cells are introduced into a circular channel and flowed in the circular path. Waste from the cells diffuses out of the droplets into the immiscible fluid. The waste from the cells is removed and fluids are replenished to approximately maintain the volume in the circular path. For example, if the droplets contain cells and cell medium, the cell medium may be replenished by coalescing with a droplet containing cell medium.

### Thermal Regulator

In another aspect of the invention, a portion of the multichannel system comprising one or more microfluidic channels is in thermal contact with a thermal regulator. The thermal regulator can be any device that regulates temperature. This includes, for example, resistive wires that heat up when a voltage is applied (such as those used in toasters), resistive heaters, fans for sending hot or cold air toward the isolated portion, Peltier devices, IR heat sources such as projection bulbs, circulating liquids or gases in a contained device, and microwave heating.

Thermal contact between a portion of the multichannel system and the thermal regulator provides thermal regulation of fluidic samples or droplets contained therein for incubation and/or regulation of biochemical or chemical reactions. The ability of the thermal regulator to be programmed for different temperatures and incubation times, together with other aspects of the invention to control the introduction of samples, reactants and other reagents into the microfluidic channels provides the ability to control reaction times, temperatures, and reaction conditions within the microfluidic channels. For example, a portion of the multichannel system is in contact with a heat spreader of the thermal regulator. There may be an air gap between the portion of the microfluidic device and the heating element of the thermal regulator. The microfluidic device can be secured to the thermal regulator by one or more bolts, screws, pins, clips, brackets, or other such securing devices.

The thermal regulator can be an electrical apparatus comprising one or more temperature sensors, e.g., thermocouples, thermistors, RTDs, and one or more regulators configured for temperature regulation, incubating, or thermal cycling a portion of an attached microfluidic device. In turn, the fluidic biological or chemical samples introduced into the portion of the microfluidic device are heated, incubated, cooled, or thermal cycled in repetitive fashion in order to carry out one or more of a number of biochemical or chemical procedures or processes.

In another embodiment, the thermal regulator is configured into separate thermal zones, or stations, and each zone comprising a separate thermal regulator, one or more temperature sensors, and a regulator dedicated to each zone for the separate thermal control of that zone. For example, an embodiment of the invention may include four individually controlled thermal zones each configured to a portion of a microfluidic device.

### Assays

The systems of the invention can be used for any process that involves manipulation and movement of small volumes of fluid (e.g., micro scale, nano scale, etc.). In certain embodiments, systems of the invention are used for chemical synthesis reactions or biological or chemical assays, such as sample preparation and analysis in a variety of fields in the art, including without limitation for many fields such as DNA sequencing, microarray sample preparation, genotyping, gene expression, biodefense, food monitoring, forensics, proteomics and cell biology. Assays involving the amplification of nucleic acids may be performed in the device of the present invention. Thus, assays applying to digital amplification techniques and multiplex PCR in droplets can be achieved using the device of the present invention. See for example, U.S. Patent Application 20110244455, herein incorporated by reference.

Numerous processes may be completed within the device of the present invention. Nucleic acids, proteins, lipids, etc. may be processed and analyzed with the present invention and methods. However, it should be appreciated that any material or species may be enveloped in a droplet and processed in the device of the invention. For example, samples may be obtained from animals (including humans) and encompass fluids, solids, tissues, and gases. Biological samples include, without limitation, cells and any components thereof, blood products, such as plasma, serum and the like, proteins, peptides, amino acids, polynucleotide, lipids, carbohydrates, and any combinations thereof. The sample may include chemicals, organic or inorganic, used to interact with the interactive material. Environmental samples include environmental material such as surface matter, soil, water, crystals and industrial samples.

Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Nucleic acid molecules may be single-stranded, double-stranded, or double-stranded with single-stranded regions (for example, stem- and loop-structures). Nucleic acid molecules can be synthetic or derived from naturally occurring sources. As known in the art, nucleic acid molecules are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Nucleic acid molecules may be obtained from a single cell. Biological samples for use in the present invention include viral particles or preparations. Nucleic acid molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention.

Nucleic acid molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA. In certain embodiments, the nucleic acid molecules are bound as to other target molecules such as proteins, enzymes, substrates, antibodies, binding agents, beads, small molecules, peptides, or any other molecule and serve as a surrogate for quantifying and/or detecting the target molecule.

Any suitable PCR methodology or combination of methodologies may be utilized in the droplet-based assays disclosed herein, such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, or universal fast walking PCR, among others.

Methods for performing polymerase chain reaction (PCR) in droplets are shown for example in Link et al. (U.S. patent application Nos. 2008/0014589, 2008/0003142, and 2010/0137163), Anderson et al. (U.S. Pat. No. 7,041,481 and which reissued as RE41,780) and European publication number EP2047910 to Raindance Technologies Inc. The content of each of which is incorporated by reference herein in its entirety. See also Brown et al. (U.S. Pat. Nos. 6,143,496 and 6,391,559) and Vogelstein et al. (U.S. Pat. Nos. 6,440,706, 6,753,147, and 7,824,889), the content of each of which is incorporated by reference herein in its entirety, relating to digital PCR (dPCR) as an alternative quantitation method in which dilute samples are divided into many separate reactions. The nucleic acids undergo the same thermal cycling and chemical reaction as the droplets passes through each thermal cycle as they flow through the channel. The total number of cycles in the device is easily altered by an extension of thermal zones or by the creation of a continuous loop structure. The sample undergoes the same thermal cycling and chemical reaction as it passes through N amplification cycles of the complete thermal device. The device and methods of the present invention have utility in droplet based digital PCR technology, as described in Link et al. (U.S. patent application Nos. 2008/0014589, 2008/0003142, and 2010/0137163), Anderson et al. (U.S. Pat. No. 7,041,481 and which reissued as RE41,780) and European publication number EP2047910 to Raindance Technologies Inc, (the contents of each of which are incorporated by reference herein in their entireties). In this assay, a library droplet is merged with a template droplet which contains all the PCR reagents including genomic DNA except for the primers. After merging of the template and the primer library droplets the new droplet contains all the reagents necessary to perform PCR. The droplet is then thermal cycled to produce amplicons.

In some embodiments of the invention, the device and methods are instrumental in a polymerase chain reaction, or other methods of analyzing nucleic acids. In this embodiment, after amplification of nucleic acids, droplets are flowed to a detection module for detection of amplification products. The droplets may be individually analyzed and detected using any methods known in the art, such as detecting the presence or amount of a reporter. Generally, the detection module is in communication with one or more detection apparatuses.

In certain embodiments, amplified target are detected using detectably labeled probes. In particular embodiments, the detectably labeled probes are optically labeled probes, such as fluorescently labeled probes. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are FAM and VIC (fluorescent label, commercially available from Applied Biosystems, Inc.). Labels other than fluorescent labels are contemplated by the invention, including other optically-detectable labels. See for example, U.S. Patent Application 20110244455.

Cell Culture Assays using Systems of the Invention

Devices and methods of the present invention have utility in the field of cell culturing. Cell culturing devices are commercially available, however, each of the currently available systems have at least, the following limitations: large size; high cost (particularly robotics); possible contamination, use of large scale cultures; treating cells with an enzyme; requiring complex software and using a large cell volume. Commercially available cell automation systems are developed by combining robotic stages, however, these systems are expensive given the cost of the robotic arms and the software used to drive them. There are a number of other automated cell systems commercially available, see for example the PANsys3000 system, which is a highly automated cell-culture system manufactured by Pan-SysTech; the CompacT SelecT system, which is an automated cell culture and assay-ready plating system manufactured by the Automation Partnership; Tecan, Inc. manufacturers several automated devices for cell culturing; Hamilton Robotics offers an extensive range of robotic systems for incorporation into cell culturing systems; and an automated cell culture system manufactured by MatriCal Bioscience.

Any type or kind of cell may be used in conjunction with the current invention. For example, induced pluripotent stem cells or iPS cells or iPSs, may be used in conjunction with the present invention. An iPS cell refers to a cell that has been reprogrammed from a somatic cell to a more pluripotent phenotype by any means of reprogramming. The culture environment within the device of the invention must be controlled and monitored to ensure the health of the cells. The specific culture conditions may vary depending on the cell type. However, most culture conditions consist of a suitable vessel containing a substrate or medium that supplies the essential nutrients (amino acids, carbohydrates, vitamins, and minerals), growth factors, hormones and gases (O₂, CO₂). Furthermore, the physicochemical environment (pH, osmotic pressure, temperature) must be regulated. Cells may be grown floating in the culture medium (suspension culture) or grown while attached to a solid or semi-solid substrate (adherent or monolayer culture).

Harvesting of cells can be accomplished by known methods in the art. Cells can be isolated from tissues for ex vivo culture in a variety of ways. For example, cells can be purified from blood; however, only the white cells are capable of growth in culture. Mononuclear cells can be released from soft tissues by enzymatic digestion with enzymes such as collagenase, trypsin, or pronase. Primary cells, cells cultured directly from a subject, have limited lifespan. After a certain number of population doublings, cells stop dividing but retain viability. There is the case of established or immortalized cell lines that have acquired the ability to proliferate indefinitely either through random mutation or deliberate modification.

Cells are grown and maintained at an appropriate temperature and gas mixture (typically, 37° C., 5% CO₂ for mammalian cells), usually in a cell incubator. Culture conditions vary widely for each cell type, and variation of conditions for a particular cell type can result in different phenotypes. See for example, Tsai et al., “Isolation of human multipotent mesenchymal stem cells from second-trimester amniotic fluid using a novel two-stage culture protocol,” Hum. Reprod. (2004) 19 (6): 1450-1456. doi: 10.1093/humrep/deh279, where amniotic fluid-derived mesenchymal stem cells (AFMSCs) were cultured to confluence and shifted to osteogenic medium (α-MEM supplemented with 10% FBS, 0.1 μmol/l dexamethason, 10 mmol/l (3-glycerol phosphate, 50 μmol/l ascorbate) and adipogenic medium (α-MEM supplemented with 10% FBS, 1 μmol/l dexamethasone, 5 μg/ml insulin, 0.5 mmol/l isobutylmethylxanthine and 60 μmol/l indomethacin) for 3 weeks. For differentiation of neural cells, AFMSCs were incubated with α-MEM supplemented with 20% FBS, 1 mmol/l(3-mercaptoethanol, 5 ng/ml bFGF (Sigma, St Louis) for 24 h, and then treated with serum depletion for 5 h.

The cell medium provides the necessary nutrients, growth factors, and hormones for cell growths, as well as regulating the pH and the osmotic pressure of the culture. The cell culture medium according to the present invention is a medium allowing for and supporting growth of the animal cells thus cultured. Growth is understood as an increase in viable cell density during at least a certain period of the cell culture. According to the present invention, such definition of ‘growth medium’ is to be understood as being opposed to the term ‘maintenance medium’ in its usual meaning in the art. A maintenance medium is a cell culture medium which supports cell viability but which does not encourage cell growth. Often, such maintenance media do not contain essential growth factors such as transferrin, insulin, albumin and the like. See for example, cell culture medium related patents: U.S. Pat. No. 4,038,139, 1977; U.S. Pat. No. 7,258,998, 2007; U.S. patent application Ser. No. 13/497,707, 2010; U.S. Pat. No. 8,338,177, 2012; and U.S. patent application Ser. No. 13/695,002, 2011.

The growth medium controls the pH of the culture and buffers the cells in culture against fluctuations in the pH. This buffering may be achieved by including an organic (e.g., HEPES) or CO₂ bicarbonate based buffer. Control of pH is needed to ensure the growth and health of cells in culture. Most normal mammalian cell lines grow well at pH 7.4, and there is very little variability among different cell strains. However, some transformed cell lines have been shown to grow better at slightly more acidic environments (pH 7.0-7.4), and some normal fibroblast cell lines prefer slightly more basic environments (pH 7.4-7.7). Because the pH of the medium is dependent on the delicate balance of dissolved carbon dioxide (CO₂) and bicarbonate (HCO₃), changes in the atmospheric CO₂ can alter the pH of the medium. Therefore, it is necessary to use exogenous CO₂ when using media buffered with a CO₂ bicarbonate based buffer, especially if the cells are cultured in open dishes or transformed cell lines are cultured at high concentrations. While most researchers usually use 5-7% CO₂ in air, 4-10% CO₂ is common for most cell culture experiments. However, each medium has a recommended CO₂ tension and bicarbonate concentration to achieve the correct pH and osmolality.

The optimal temperature for cell culture largely depends on the body temperature of the host from which the cells were isolated, and to a lesser degree on the anatomical variation in temperature (e.g., temperature of the skin may be lower than the temperature of skeletal muscle). Overheating is a more serious problem than under heating for cell cultures; therefore, often the temperature in the incubator is set slightly lower than the optimal temperature. Most human and mammalian cell lines are maintained at 36° C. to 37° C. for optimal growth. Insect cells are cultured at 27° C. for optimal growth; they grow more slowly at lower temperatures and at temperatures between 27° C. and 30° C. Above 30° C., the viability of insect cells decreases, and the cells do not recover even after they are returned to 27° C. Avian cell lines require 38.5° C. for maximum growth. Although these cells can also be maintained at 37° C., they will grow more slowly. Cell lines derived from cold-blooded animals (e.g., amphibians, cold-water fish) tolerate a wide temperature range between 15° C. and 26° C. The consequences of deviating from the culture conditions required for a particular cell type can range from the expression of aberrant phenotypes to a complete failure of the cell culture.

Subculturing, or passaging, is the removal of the medium and transfer of cells from a previous culture into fresh growth medium, a procedure that enables the further propagation of the cell line or cell strain. Traditionally, to keep the cells at an optimal density for continued growth and to stimulate further proliferation, the culture has to divided and fresh medium supplied. For example, subculturing could be needed if a drop in pH is observed. A drop in the pH of the growth medium usually indicates a buildup of lactic acid, which is a by-product of cellular metabolism. Lactic acid can be toxic to the cells, and the decreased pH can be sub-optimal for cell growth.

As cells generally continue to divide in culture, they generally grow to fill the available area or volume. This can generate several issues: nutrient depletion in the growth media; changes in pH of the growth media; and accumulation of apoptotic/necrotic (dead) cells. Cell-to-cell contact can stimulate cell cycle arrest, causing cells to stop dividing, known as contact inhibition. Cell-to-cell contact can also stimulate cellular differentiation. Genetic and epigenetic alterations, with a natural selection of the altered cells potentially leading to overgrowth of abnormal, culture-adapted cells with decreased differentiation and increased proliferative capacity. Therefore, processing of the cells to ensure removal of harmful species and replenishment of cell medium is needed every 1-3 days, depending on the particular protocol.

Traditionally, cell viability is determined by staining the cells with trypan blue. As trypan blue dye is permeable to non-viable cells or death cells whereas it is impermeable to this dye. Stain the cells with trypan dye and load to haemocytometer and calculate % of viable cells. Cell viability is calculated as the number of viable cells divided by the total number of cells within the grids on the hemacytometer. If cells take up trypan blue, they are considered non-viable. It would be appreciated by one skilled in the art for the optical detection of cells containing trypan blue.

Cell culture entails growing cells in a growth medium under controlled temperature and atmosphere conditions. In the present invention, cells are encapsulated in droplets containing cell medium. The cells can be flowed from various chambers via the multichannel system of the invention. Microfluidic channels can be aligned to cause flow of the droplets into a humidifier chamber. For example, mammalian cells are grown in humidified atmosphere at 37° C. and 5% CO₂, in cell culture incubators. Microfluidic channels can be aligned to deliver CO₂ to the humidifier chamber within the multichannel system by aligning microfluidic channels that seal, or by an inlet microfluidic channel configured to deliver liquids or gases to the chamber. It should be appreciated that alignment of microfluidic channels of the invention can be sealed to allow for the transport of gases. To replenish or re-suspend the cells in fresh growth medium, which could be required every 2-3 days, the droplets are flowed from a chamber into the microfluidic channels to be merged or coalesced with fresh growth medium. It should be appreciated that droplets containing cells during culturing may be retained within microfluidic channels or chambers of the present invention. After merging or coalesced with fresh growth medium, the droplets may be retained within microfluidic channels, or the cells may be diverted to a chamber.

In an embodiment of the invention, cells are grown in nanoliter-microliter droplets in cell medium that is replenished every 2-3 days. In some assays, cells may require splitting every 2-3 days. Media change involves adding one or more droplets of fresh media to a droplet of incubated cells and thereby partially replenishing growth media. Merging of droplets is discussed above. Cells are further incubated in the combined droplet or in smaller droplets generated by splitting the combined droplet. Cell subculture or splitting is achieved similarly to media change by combining (merging and mixing) a droplet of incubated cells and a droplet of fresh media, splitting the combined droplet, and repeating this procedure using the split droplet(s) until a desired cell concentration is reached. Final droplets are then incubated, while other droplets of suspended cells generated in the subculturing process are discarded. Incubation can be accomplished within the microfluidic channels of the device, or in chambers of the device. One or more thermal regulators can be employed to ensure proper temperature.

In a multiplexed assay, multiple droplets containing one kind or multiple kinds of cells are exposed to droplets containing one or multiple reagents and are assayed similarly to the assays described above. A multiplex device can also be used for multiplex cell culture, where cells can be grown and maintained in multiple droplets.

There are several ways of configuring the chambers for housing the droplets. In one configuration the chambers are external to the microfluidic device. Alternatively, the chambers could be integrated into the microfluidic device, and are in flow communication with the microfluidic channels of the microfluidic device. Signals from secondary droplets are detected using multiplexed detection instruments such as optical sensors, optical detectors comprising a light source and a photodetector, optical detectors that measure absorbance, fluorescence, epifluorescence, chemiluminescence, UV light detector, radiometric detector, scanning, imaging, and confocal microscopy detectors, CCD cameras, and microplate readers. The detection step is to detect or identify any reaction products formed by the cell assay, or to identify, monitor and count the cells if a cell culture is being performed to mention just a few. All waste liquid droplets generated during the assay are directed to the waste chamber. Chambers may contain wash solutions for flushing the microfluidic channels of the device between assays.

The devices and methods of the present invention have utility in the area of cell culturing. A multichannel device of the invention can be employed to culture cells in a controlled and stable environment. For example, FIG. 6 depicts a multichannel device for manipulating and directing droplets. As shown in FIG. 6, microfluidic channels 601, 602, 603, 604, and 605 contain droplets. In a cell culture assay, the droplets 606 contain cells and cell medium to ensure the health and proliferation of the cells. Microfluidic channel 600 contain cell medium. To add cell medium to the droplets, microfluidic channel 607 aligns with microfluidic channel 600 for a span of time to create a droplet of cell medium. The cell medium droplet merges with a cell droplet 606, as discussed above. Microfluidic channel 607 aligns with microfluidic channel 609 to allow the droplets containing cells and cell medium to flow into microfluidic channel 609. It should be appreciated that waste chamber 611 may align with microfluidic channels 601, 602, 603, 604, and 605 to remove droplets containing non-viable cells. It should also be appreciated that chamber 617 may align with microfluidic channel 609 to collect the droplets containing cells for detection, assay, or incubation. Droplets that are not diverted flow from microfluidic channel 609 into microfluidic channel 619 when microfluidic channel 615 is positioned therebetween. It should also be appreciated that chambers 623 and 625 can align with microfluidic channel 619 to divert droplets into chambers 623 and 625. It should be appreciated that chambers 623 and 625 may be to collect droplets containing non-viable cells or to incubate the cell containing droplets.

In an alternative embodiment, droplets containing cells may be merged or combined with compounds for investigation of reactivity and efficacy. For example, FIG. 6 depicts a multichannel device for manipulating and directing droplets. As shown in FIG. 6, microfluidic channels 601, 602, 603, 604, and 605 contain droplets. In a cell investigation assay, the droplets 606 contain cells and cell medium to ensure the health and proliferation of the cells. Microfluidic channel 600 contain a fluid comprising a test compound. It should be appreciated that any test compound may be used in the assay. To add the target compound to the droplets, microfluidic channel 607 aligns with microfluidic channel 600 for a span of time to create a droplet containing the target compound. The target compounddroplet merges with a cell droplet 606, as discussed above. Microfluidic channel 607 aligns with microfluidic channel 609 to allow the droplets containing cells and target compound to flow into microfluidic channel 609. It should be appreciated that waste chamber 611 may align with microfluidic channels 601, 602, 603, 604, and 605 to remove droplets containing non-viable cells. It should also be appreciated that chamber 617 may align with microfluidic channel 609 to collect the droplets containing cells for detection, assay, or incubation. Droplets that are not diverted flow from microfluidic channel 609 into microfluidic channel 619 when microfluidic channel 615 is positioned therebetween. It should also be appreciated that chambers 623 and 625 can align with microfluidic channel 619 to divert droplets into chambers 623 and 625. It should be appreciated that chambers 623 and 625 may be to collect droplets containing non-viable cells or to incubate the cell containing droplets.

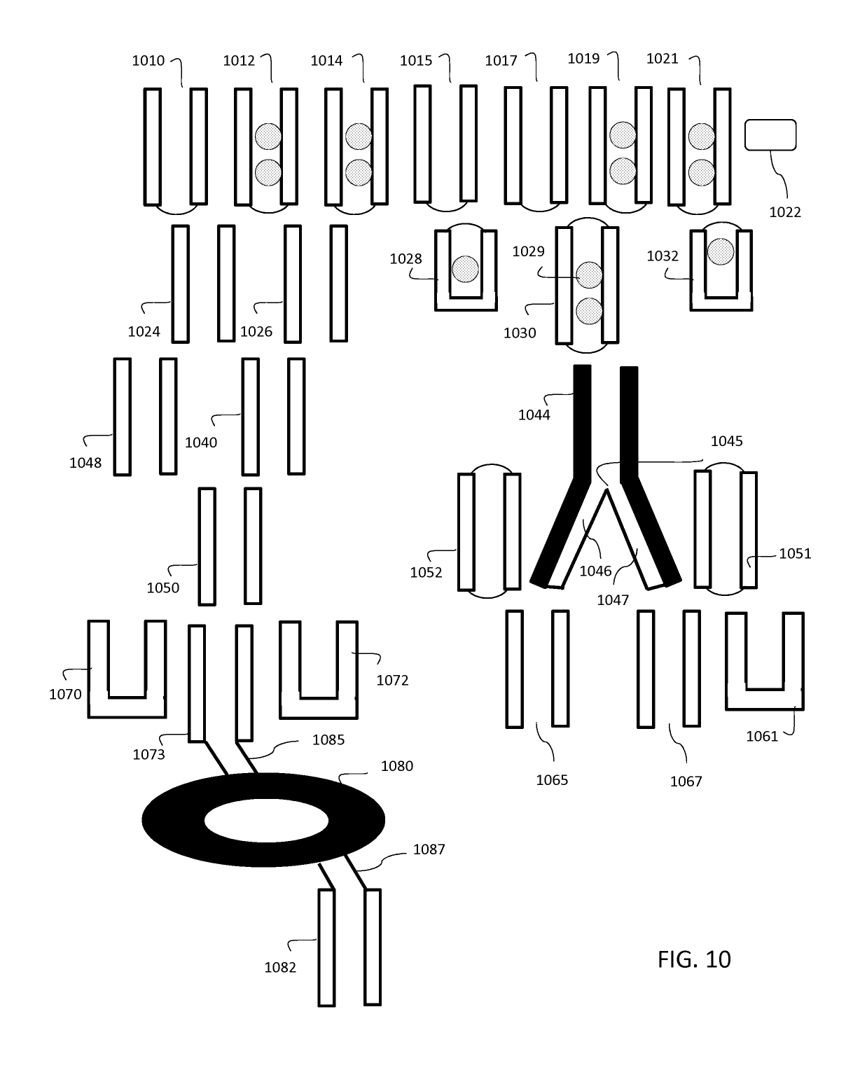

A multichannel system is depicted in FIG. 10. In regards to FIG. 10, the microfluidic channels and chambers may be aligned by sliding the microfluidic channels proximately to one another. Microfluidic channels may be slid together or independently of one another, as discussed above. Chambers may also be slid in together, or independently. Microfluidic channels that are slid together may be located on the same substrate, or may be located on different substrates. Similar arrangement can be with chambers. As discussed above, alignment may create air gaps (not shown). The liquid in the channels or chambers bridge the air gap to allow fluid and/or droplets to flow from a microfluidic channel to another microfluidic channel, or from a microfluidic channel to a chamber. As shown in FIG. 10, multiple microfluidic channels 1010, 1012, 1014, 1015, 1017, 1019, and 1021 contain droplets, fluids, or fluids containing target compounds. A detector 1022 is positioned proximate to microfluidic channel 1021. A detector can be positioned proximate to any microfluidic channel in the multichannel device. In an aspect of the invention, cells may be combined with target compounds to test or analyze the effects on cells. As depicted in FIG. 10, chamber 1028 receives a droplet from any of microfluidic channels 1010, 1012, 1014, 1015, 1017, 1019, or 1021. Chamber 1028 can be for waste or for incubating the droplet for a span of time. Similarly, chamber 1032 can receive a droplet from any of microfluidic channels 1010, 1012, 1014, 1015, 1017, 1019, or 1021. Chamber 1032 can be for waste, or to incubate the cells for any span of time. A heating element or heating source, not shown, may be proximately located to either chambers 1028 and 1032. As shown in FIG. 10, microfluidic channel 1030 received a droplet 1029 from any of microfluidic channels 1010, 1012, 1014, 1015, 1017, 1019, or 1021. Droplet 1029 passes through microfluidic channel 1030 and is then passed to microfluidic channel 1044. Microfluidic channel 1044 contains a branch and an obstruction at 1045. As stated above, the obstruction 1045 caused cells to be split. A droplet introduced into microfluidic channel 1044 would be split at obstruction 1045, causing part of the droplet to be directed down microfluidic channel 1046 and the other part of the droplet is directed down microfluidic channel 1047. Microfluidic channels 1052 and 1051 may contain fluid which contains cell medium, sample fluid, reactants, target compounds, etc. Microfluidic channel 1065 can align with microfluidic channel 1046 to receive a droplet from microfluidic channel 1046. Microfluidic channel 1065 can align with microfluidic channel 1052 to accept fluid, and can form a droplet from the fluid in microfluidic channel 1052. As discussed earlier, a droplet from microfluidic channel 1046 and a formed droplet from microfluidic channel 1052 can merge. This technique allows for the fluid from microfluidic channel 1052 to be combined with droplets from 1046. Alternatively, droplets from microfluidic channel 1046 can pass to microfluidic channel 1065 and not be merge with any other fluid. From microfluidic channel 1065, droplets can be further processed. Similarly, droplets from microfluidic channel 1047 can flow into microfluidic channel 1067 by the alignment between microfluidic channels 1047 and 1067. Microfluidic channel 1067 can align with microfluidic channel 1051 to accept fluid, by forming a droplet, and can causing coalescing between a droplet from microfluidic channel 1047 and microfluidic channel 1067. Chamber 1061 can align with microfluidic channels 1046 or 1047 to collect droplets as waste or to incubate the droplets.

As shown in FIG. 10, microfluidic channels 1024 and 1026 can receive fluids and/or droplets from any of microfluidic channels 1010, 1012, 1014, 1015, 1017, 1019, or 1021. For example, microfluidic channel 1024 can align with microfluidic channel 1012 to receive a droplet. Microfluidic channel 1024 can then align with microfluidic channel 1010 to form a droplet from the fluid in microfluidic channel 1010. The fluid can contain reactants, cell medium, or target compounds. In microfluidic channel 1024, droplets from microfluidic channels 1010 and 1012 can coalesce. Similarly, microfluidic channel 1026 can receive droplets and fluids from any of microfluidic channels 1010, 1012, 1014, 1015, 1017, 1019, or 1021. For example, microfluidic channel 1026 can receive droplets from microfluidic channel 1014 and can form droplets from the fluid contained in microfluidic channel 1015. The droplets from microfluidic channels 1014 and 1015 can be coalesced by techniques and methods discussed above. By way of example, microfluidic channel may contain target compounds that are combined with droplets in microfluidic channel 1012. Microfluidic channel 1040 can align with microfluidic channels 1024 and 1026 to receive the droplets contained within microfluidic channels 1024 and 1026. Microfluidic channel 1050 can align with microfluidic channels 1040 or 1048. Microfluidic channel 1048 can contain fluid, which contains reactants, cell medium, or target compounds. By aligning with microfluidic channel 1048, microfluidic channel 1050 can create droplets from the fluid contained within microfluidic channel 1048. Droplets from microfluidic channel 1040 and 1048 can be coalesced in microfluidic channel 1050 by the methods and techniques discussed above. Droplets in microfluidic channel 1050 can be diverted into microfluidic channel 1073 by aligning microfluidic channels 1050 and 1073. Droplets in microfluidic channel 1050 can be diverted to chamber 1070, which can be waste chamber. Droplets in microfluidic channel 1050 can be diverted to chamber 1072, which can be an incubation chamber. Droplets in microfluidic channel 1073 are diverted into circular microfluidic channel 1080. Droplets may circulate via a roter, not shown. Droplets may exit circular microfluidic channel 1080 via outlet 1087 and into microfluidic channel 1082.

For example, in a preferred embodiment, cells are encased in the droplets contained in microfluidic channels 1019 and 1021. Microfluidic channel 1030 aligns with microfluidic channel 1019 to receive droplets. The droplets are flowed into microfluidic channel 1044 to split the droplets, and thereby split the cells by aligning microfluidic channels 1044 and 1030. Droplets, and thereby cells, are split and flow into microfluidic channels 1046 and 1047. Microfluidic channel 1065 aligns with microfluidic channel 1046 and then microfluidic channel 1065 aligns with microfluidic channel 1052 to cause droplets from 1046 and 1052 to be closely positioned to allow for passive merging. The fluid in microfluidic channel 1052 is cell medium, so that passive merging causes replenishing of cell medium to the droplets. The same procedure is repeated for the droplets in microfluidic channel 1047. Following splitting and replenishing of cell medium, the droplets are incubated in a chamber (not shown) by flowing the droplets from microfluidic channels 1065 and 1067 into a chamber.

For example, in another preferred embodiment, droplets in microfluidic channel 1012 contain cells, and microfluidic channel 1010 contains a fluid containing a testing compound, (e.g., a drug molecule). Microfluidic channel 1024 aligns with microfluidic channel 1012 to receive a droplet and aligns with microfluidic channel 1010 to flow a small volume of the fluid in microfluidic channel 1024. The droplet and small volume are positioned closely to cause passive merging, as discussed above. Microfluidic channel 1048 aligns with microfluidic channel 1024 to receive a coalesced droplet and aligns with microfluidic channel 1026 to receive a small volume of the fluid contained in microfluidic channel 1026. The fluid in microfluidic channel may be cell medium, reactants, nutrients, buffer, etc. Microfluidic channel 1050 aligns with microfluidic channel 1048 to direct the droplets to circular microfluidic channel 1080 via microfluidic channel 1073. The droplets are circulated for a period of time and then diverted to microfluidic channel 1087. Droplets are then flowed into a chamber (not shown) for detection.

In an aspect of the invention, combinatorial methods may be employed in assays using the device of the invention. Any combinatorial approach or strategy known in the art may be used with systems of the invention. FIG. 11 provides an exemplary embodiment, showing that droplets may be organized into groups, called Words. A Word, as used herein, is a plurality of n droplets. Each droplet is a letter in the Word. For example, a Word comprising five droplets would correlate to a Word of five letters. For example, if a Word contains three droplets, and each droplet represents a letter, then the Word may be arranged as aaa, bbb, ccc, abc, abb, acc, acb, etc. In FIG. 11, four Words 1105 are contained within a microfluidic channel 1103. Each Word 1105 comprises four droplets 1107, or four letters 1107. Each letter, or droplet in Word 1103 is the same and may, for example, be represented as aaaa. As shown in FIG. 11, four Words 1115 comprise four droplets, or four letters, of three droplets 1107 and one droplet 1117 (represented, for example, as aaab). Droplet 1117 is formed by mixing a droplet 1107 with additional materials, reactants, etc. Mixing or coalescing of droplets is described above. As shown in FIG. 11, microfluidic channel 1123 contains three Words 1115, as one Word 1115 was directed to waste, methods of which are described above. As shown in FIG. 11, three Words 1125 are contained in microfluidic channel 1133. Each Word 1125 contains two droplets of 1107, one droplet 1117, and one droplet 1127 (represented, for example, as aacb). Droplet 1127 can be formed by mixing droplet 1107 with additional materials, reactants, etc. Mixing or coalescing of droplets is described above. In microfluidic channel 1143, contains two Words 1125, where one Word 1125 was directed to waste, methods of which are described above. Microfluidic channel 1153 contains two Words 1135, in which each Word 1135 contains one droplet 1107, one droplet 1117, one droplet 1127, and one droplet 1137 (represented, for example, as adcb). Droplet 1137 can be formed by mixing droplet 1107 with additional materials, reactants, etc. Microfluidic channel 1163 contains one Word 1135.

FIG. 11 depicts a work flow process in which Words are processed through a microfluidic device of the invention. Microfluidic channel 1103 contains Words 1105, in which each Word 1105 contains similar droplets 1107. The droplets 1107 may contain any components or materials described herein. For example, each droplet 1107 may contain a cell. The Words 1105 are flowed into microfluidic channel 1113, and a droplet 1107 is coalesced with another droplet to form droplet 1117. For example, a cell in a droplet 1107 may be merged or mixed with a droplet containing a target compound). The Words 1115 are then flowed into microfluidic channel 1123, however, one Word was diverted to waste, thereby only three Words were flowed into microfluidic channel 1123. As described above, droplets, or Words may be diverted to waste by aligning microfluidic channel 1113 with a waste chamber. For example, a detector may have detected a deficiency, error, or abnormality, causing the diversion to waste. Words 1115 are flowed into microfluidic channel 1133 and a droplet 1107 is coalesced with another droplet to form droplet 1127, forming Word 1125. For example, a droplet 1107 may be merged with a droplet containing the same or different target compound, a reactant, or any other species. Words 1125 are flowed into microfluidic channel 1153, however, one Word 1125 was diverted to waste, thereby only two Words were flowed into microfluidic channel 1143. As described above, an abnormality may have been detected and the Word was diverted into a waste chamber. Words 1125 are flowed into microfluidic channel 1153 and a droplet 1107 is coalesced with another droplet to form droplet 1137, forming Word 1135. The droplet merged with droplet 1107 may contain the same or different target compound, reactants, or any other material. Words 1135 are then flowed into microfluidic channel 1163, however, one Word was diverted to waste, thereby only one Word 1135 was flowed into microfluidic channel 1163. As stated above, the diversion could have been based upon the detection of an abnormality. In an aspect of the invention a droplet, or letter, may contain an identifying tag to identify the Word.

In an exemplary embodiment of the invention, iPS cells are transformed from somatic cells, cultured, and used in expression profiling employing devices of the present invention. Any method known in the art may be employed to transform somatic cells into iPS cells. In certain embodiments, an indirect route that involves dedifferentiation and then redifferentiation of the somatic cells is employed. Such a route involves reprogramming a variety of somatic cell types from different lineages to produce a dedifferentiated embryonic stem cell state. Indirect routes include somatic cell nuclear transfer, cell fusion, or creation of induced pluripotent stem cells by introduction of genes such as Oct4. The dedifferentiated cells are then redifferentiated to target cells along respective mesodermal, endodermal, or ectodermal lineages. Further description of such methods are found for example in Isacson et al. (U.S. patent application No. 2010/0021437), Yamanaka et al. (U.S. patent application No. 2009/0047263), Sakurada et al. (U.S. patent application No. 2009/0191159), Yamanaka et al. (U.S. patent application No. 2009/0227032), Sakurada et al. (U.S. patent application No. 2009/0304646), Sakurada et al. (U.S. patent application No. 2010/0105100), Takahashi et al. (U.S. patent application No. 2010/0105137), Sakurada et al. (U.S. patent application No. 2010/0120069), Sakurada et al. (U.S. patent application No. 2010/0267135), Hochedlinger et al. (U.S. patent application No. 2010/0062534), and Hochedlinger et al. (U.S. patent application No. 2010/0184051), the content of each of which is incorporated by reference herein in its entirety. Methods for preparing induced pluripotent stem cells by using a nuclear reprogramming factor are described in International publication number WO 2005/80598, the content of which is incorporated by reference herein in its entirety.

Employing the methods discussed herein, iPS cells are encased in droplets containing cell medium and related growth factors. The iPS cell droplets are cultured by techniques and methods discussed above, or known in the art. iPS cell droplets can be merged with other materials and reactants, including maintenance medium, transfection reagents, etc. during the incubation process.

Overall, the process of inducing cells to become multipotent or pluripotent is based on forcing the expression of polypeptides, particularly proteins that play a role in maintaining or regulating self-renewal and/or pluripotency of ES cells. Examples of such proteins are the Oct3/4, Sox2, Klf4, and c-Myc transcription factors, all of which are highly expressed in ES cells. Forced expression may include introducing expression vectors encoding polypeptides of interest into cells (Hochedlinger et al., U.S. patent application No. 2010/0062534), transduction of cells with recombinant viruses, introducing exogenous purified polypeptides of interest into cells, contacting cells with a non-naturally occurring reagent that induces expression of an endogenous gene encoding a polypeptide of interest (e.g., Oct3/4, Sox2, Klf4, or c-Myc), or any other biological, chemical, or physical means to induce expression of a gene encoding a polypeptide of interest (e.g., an endogenous gene Oct3/4, Sox2, Klf4, or c-Myc). Some basic steps to induce the cells are shown in Sakurada et al. (U.S. patent application No. 2009/0191159). These steps may involve: collection of cells from a donor, e.g., a human donor, or a third party; induction of the cells, e.g., by forcing expression of polypeptides such as Oct3/4, Sox2, Klf4, and c-Myc (110); identifying multipotent or pluripotent stem cells; isolating colonies; and optionally, storing the cells. Interspersed between all of these steps are steps to maintain the cells, including culturing or expanding the cells. In addition, storage of the cells can occur after many steps in the process. Cells may later be used in many contexts, such as therapeutics or other uses.

iPS cell droplets may be used in expression profiling where target compounds are introduced into the droplets by methods disclosed herein. In order to use expression analysis for disorder diagnosis, a threshold of expression is established. The threshold may be established by reference to literature or by using a reference sample from a subject known not to be afflicted with the disorder. The expression may be over-expression compared to the reference (i.e., an amount greater than the reference) or under-expression compared to the reference (i.e., an amount less than the reference). In expression profiling, the iPS cell droplets are merged with droplets containing target compounds and allowed to further incubate, which may involve splitting of droplets, i.e. splitting of cell cluster, and merging of droplets, i.e. introduction of freshcell medium. Methods of the invention may be used to detect any disorder or compound effect. The iPS cell droplets may be flowed passed a detector to screen for abnormalities, or diverted to a collection chamber for analysis.

The invention generally relates to microfluidic devices that include channels that are slidable relative to each other and methods of use thereof.

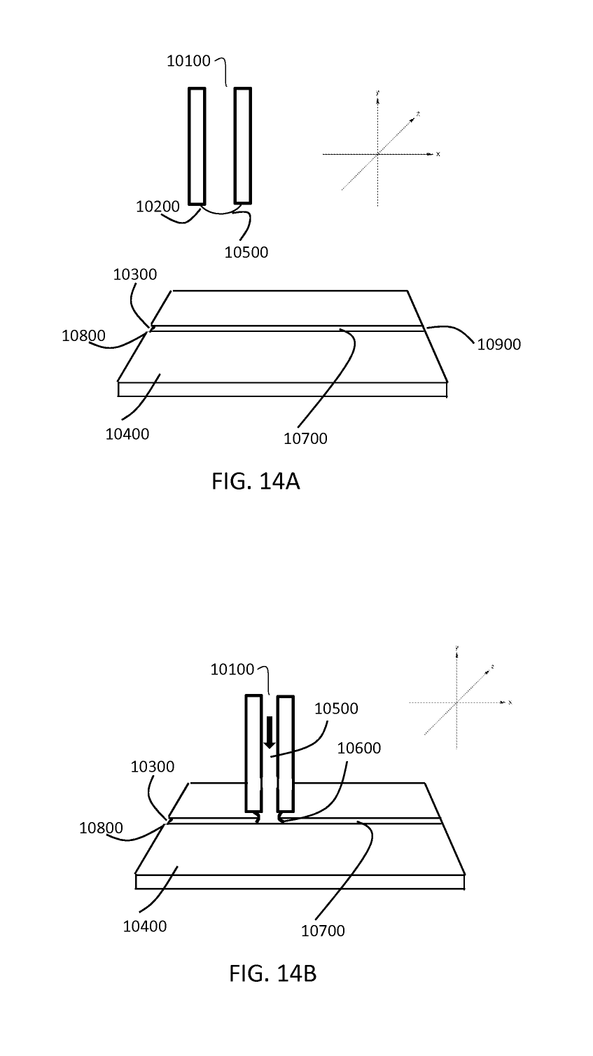

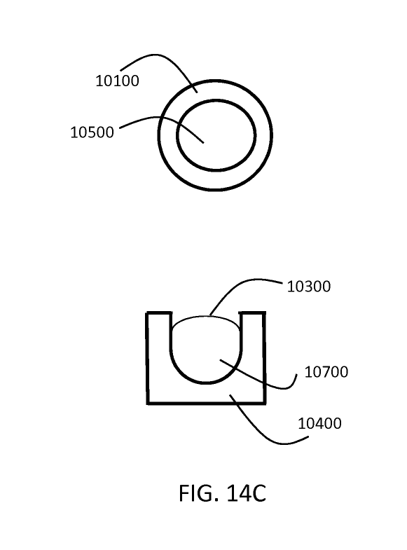

FIGS. 14A-14B show an exemplary embodiment of a microfluidic system 10000 of the invention. FIGS. 14A-14B are described in the context of two channels, one substantially vertical and the other substantially horizontal, for the sake of simplicity. However, the skilled artisan will recognize that the invention is not limited to two channels, and the invention encompasses systems designed with any number of channels, as will be described in additional embodiments below.

Microfluidic system 10000 includes a first closed channel 10100 containing a fluid 10500, having an open end 10200. The first closed channel 10100 has a cross sectional geometry that is completely circular, as shown in FIG. 14C and second open channel 10300 has a cross sectional geometry that is partially circular, also shown in FIG. 14C. The second channel 10300 is an open channel (hereafter referred to as second open channel) within a substrate 10400. Second open channel 10300 has open ends 10800 and 10900, and contains fluid 10700. The first closed channel and second open channel are slidable relative to each other such that when the open end 10200 of the first closed channel 10100 and a portion of the second open channel 10300 are aligned with each other, fluid 10500 flows from the first closed channel 10100 into the second open channel 10300 (FIG. 14B, flow shown by large downward pointing arrow within channel 10300). When the first closed channel 10100 and the second open channel 10300 are not aligned, fluid 10500 does not flow within the first closed channel 10100 and the second open channel 10300 (FIG. 14A). It should be appreciated that ends 10800 and 10900 may additionally be closed at either end. For example, end 10900 may be closed, causing flow only towards end 10800.

Alignment includes complete alignment, partial alignment, and misalignment. In complete alignment, the menisci of the two microfluidic channels are in intact and the center axes of the microfluidic channels are in alignment. In partial alignment, the center axes are not aligned, however, there is partial overlap of the first and second channels such that the distance between the center axes is sufficiently small so that flow between the two microfluidic channels occurs. In complete misalignment, there is no overlap between the channels and the distance between the center axes is sufficiently great so that flow between the two microfluidic channels does not occur. In the present invention, alignment is meant to encompass both complete and partial alignment. The device of the invention flows fluid between two microfluidic channels even in the cases of partial alignment.

The channels may slide in any direction relative to each other, e.g., horizontally, vertically, diagonally, etc. In certain embodiments, the first closed channel 10100 and the second open channel 10300 are slideable relative to each other as shown in FIGS. 14A-14B. For example, as shown in FIGS. 14A-14B, second open channel 10300 may be slidable in any direction in the x-y-z three-dimensional space, while first closed channel 10100 remains stationary. However the invention is not limited to such a configuration. In other embodiments, it may be first closed channel 10100 that is slidable in any direction in the x-y-z three-dimensional space relative to second open channel 10300, which remains stationary. In another embodiment, both the first closed channel 10100 and the second open channel 10300 are slidable in any direction in the x-y-z three-dimensional space.

In certain embodiments, the open end 10200 of the first closed channel 10100 and second open channel 10300 are exposed to atmospheric pressure. In such embodiments, the first closed channel 10100 and second open channel 10300 may be arranged in relation to each other such that an air gap 10600 exists between the channels. As shown in FIGS. 14A-14B, when the open end 10200 of the first closed channel 10100 and the second open channel 10300 are aligned with each other, fluid 10500 from the first closed channel 10100 bridges the air gap 10600 and enters the second open channel 10300.

In an aspect of the invention, the air gap may comprise any known gas, at any temperature and pressure. The air gap may be at atmospheric pressure and be comprised of air. However, the air gap is not limited to atmospheric pressure or air. In some embodiments, the devices of the present invention may be completely or partially enclosed within a chamber and the chamber may be filled with a gas other than air. The pressure can be above or below atmospheric pressure and the temperature can be at, above, or below room temperature, which is about 37 degrees Celsius. Systems of the invention can be equipped with any type of flow driving mechanism, such as pumps. In particular embodiments, gravitational force is used to produce and control flow within the system. As shown in FIGS. 14A-14B, the first closed channel 10100 and the second open channel 10300 are arranged such that gravity causes flow of fluid 10500 within the first closed channel 10100 and second open channel 10300 when the open end 10200 of the first closed channel 10100 and a portion of the second open channel 10300 are aligned with each other.

The first closed channel 10100 and second open channel 10300 may be configured such that when they are not aligned, fluid 10500 does not flow within the first closed channel 10100 and/or second open channel 10300. That can be achieved in numerous different ways, such as by adjusting length of the channels, internal diameter of the channels, viscosity of the fluid(s) within the channels, surface tension of the fluid(s) within the channels, and/or density of the fluid(s) within the channels.

### Channels

The microfluidic systems of the present invention include channels that form the boundary for a fluid. A channel generally refers to a feature on or in the system (sometimes on or in a substrate) that at least partially directs the flow of a fluid. In some cases, the channel may be formed, at least in part, by a single component, e.g., an etched substrate or molded unit. The channel can have any cross-sectional shape, for example, circular, oval, triangular, irregular, square or rectangular (having any aspect ratio), or the like, and can be covered or uncovered (i.e., open to the external environment surrounding the channel). As used herein, a closed channel has a cross sectional geometry that is completely circular, as for example, closed channel 10100 in FIG. 14C. As used herein, an open microfluidic channel refers to a microfluidic channel that is uncovered, having a lower portion and side portions, but lacking an upper portion, as for example, open channel 10300 in FIG. 14C. In embodiments in which the channel is completely covered, at least one portion of the channel can have a cross-section that is completely enclosed, and/or the entire channel may be completely enclosed along its entire length with the exception of its inlet and outlet.

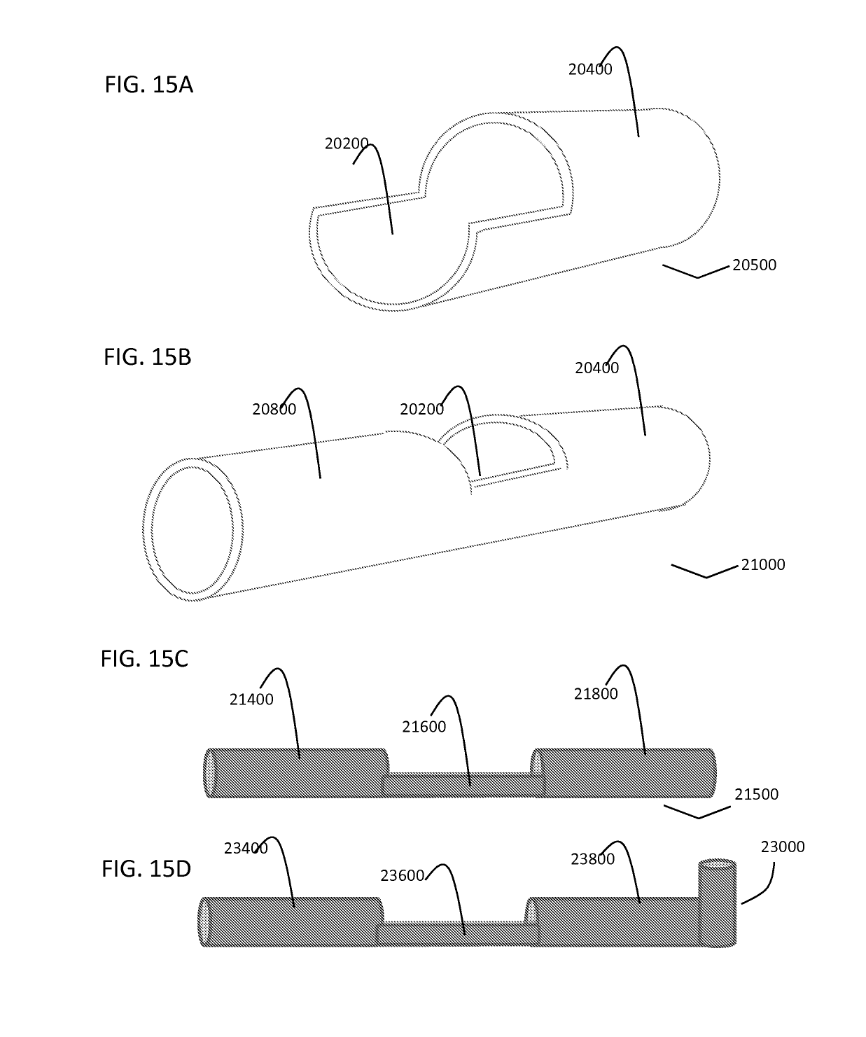

The microfluidic channels of the invention may include hybrid channels. A hybrid channel has a portion of the microfluidic channel that is closed (or covered) and a portion that is open (or uncovered). A hybrid channel may also include a channel that has two or more portions that are open. A hybrid channel may also include a channel that has two or more portions that are closed. FIGS. 15A-15D depict exemplary examples of hybrid channels. FIG. 15A depicts a hybrid channel 20500 with an open channel portion 20200 and a closed channel portion 20400. FIG. 15B depicts a hybrid channel 21000 with a first closed portion 20800, a first open channel portion 21000 and a second closed channel portion 21200. FIG. 15C depicts a first open channel portion 21400, a first closed channel portion 21600 and a second open channel portion 21800. Hybrid channels 20500, 21000, and 21500 are exemplary, and the skilled artisan would appreciate that any arrangement of open channel portions and closed channel portion may comprise a hybrid channel. It should also be appreciated that the open channel can receive fluid from a microfluidic channel at any open portion. For example, in FIG. 15B, open channel 21000 can align with a microfluidic channel at either end, and at open portion 20200. FIG. 15D depicts a hybrid channel with closed portions 23400 and 23800, and open portion 21600. FIG. 15D also depicts a vertical portion 23000 that can also align with another microfluidic channel.

A channel, closed or open, generally will include characteristics that facilitate control over fluid transport, e.g., structural characteristics (an elongated indentation) and/or physical or chemical characteristics (hydrophobicity vs. hydrophilicity) and/or other characteristics that can exert a force (e.g., a containing force) on a fluid. The fluid within the channel may partially or completely fill the channel. In some cases the fluid may be held or confined within the channel or a portion of the channel in some fashion, for example, using surface tension. In an article or substrate, some (or all) of the channels may be of a particular size or less, for example, having a largest dimension perpendicular to fluid flow of less than or equal to about 5 mm, less than or equal to about 2 mm, less than or equal to about 1 mm, less than or equal to about 500 microns, less than or equal to about 200 microns, less than or equal to about 100 microns, less than or equal to about 60 microns, less than or equal to about 50 microns, less than or equal to about 40 microns, less than or equal to about 30 microns, less than or equal to about 25 microns, less than or equal to about 10 microns, less than or equal to about 3 microns, less than or equal to about 1 micron, less than or equal to about 300 nm, less than or equal to about 100 nm, less than or equal to about 30 nm, or less than or equal to about 10 nm or less in some cases. Of course, in some cases, larger channels, tubes, etc. can be used to store fluids in bulk and/or deliver a fluid to the channel. In some embodiments, the channel is a capillary.

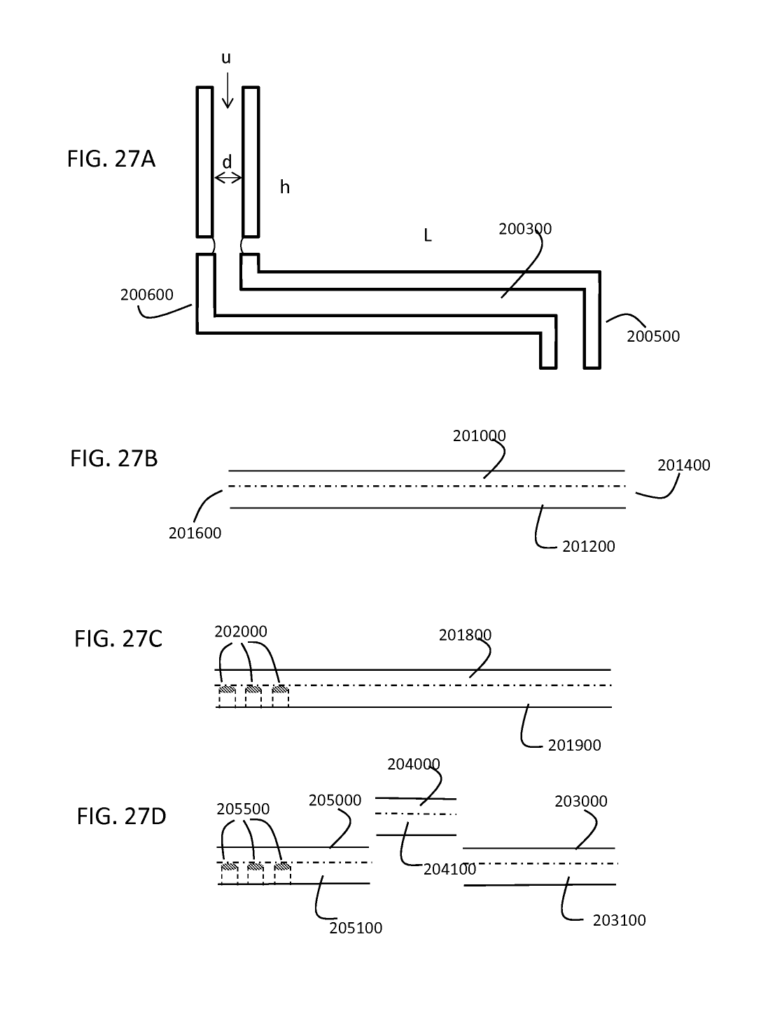

The microfluidic channels of the invention, including open microfluidic channels, are configured such that liquid is retained within the microfluidic channel when it is completely out of alignment with another microfluidic channel (e.g., no overlap between open ends of channels). Liquid may be retained within the microfluidic channel due to surface tension. The flow in a microfluidic channel system, as shown in FIG. 27A, with a height of h, an internal diameter of d, a length of L, a fluid velocity of u, a fluid density of ρ, gravitation force of g, fluid viscosity of μ, and surface tension of γ, can be represented by the equation:

$$2 ρ gh = \frac{γμ u ( 2 h + L )}{{( \frac{d}{2} )}^{2}}$$

or, rearranged as:

$$u = \frac{ρ {ghd}^{2}}{2 γμ ( 2 h + L )}$$

When fluid does not flow in the system, at maximum height, the equation becomes h=μγ/dρg.

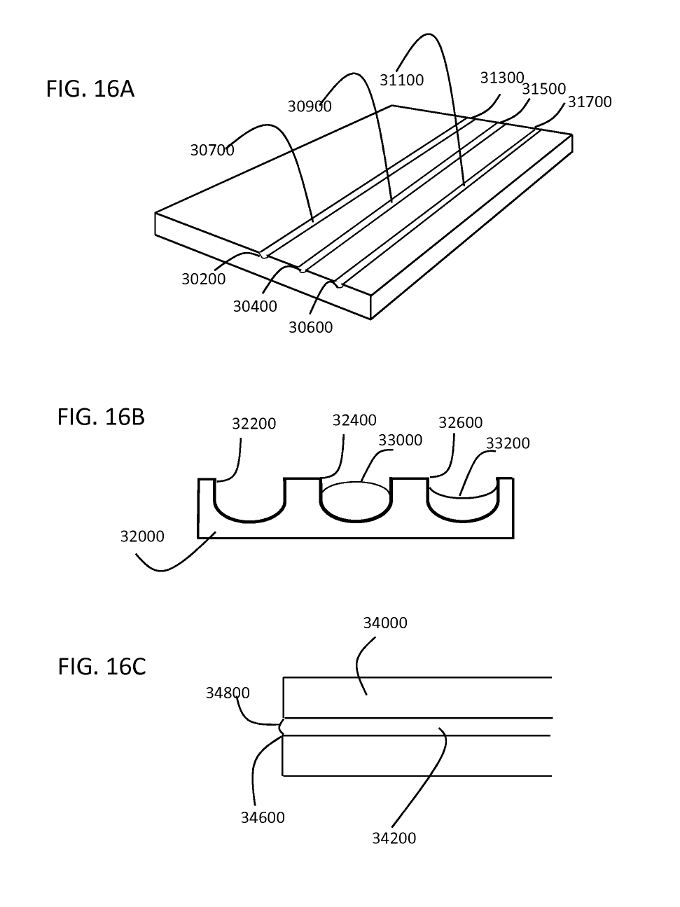

Substrates may contain one or more open microfluidic channels. FIGS. 16A and 16B depict multiple open channels in a substrate. As shown in FIG. 16A, a substrate 30500 contains multiple open microfluidic channels 30700, 30900, and 31100. The channels have ends 31300, 31500, 31700, 30200, 30400, and 30600. It should be appreciated that one or both ends of an open channel can be open. For example, microfluidic channel 30700 can have end 31300 sealed and end 30200 open, and vice versa. Similarly, microfluidic channel 30700 can have end 31300 open and end 30200 open. FIG. 16B depicts a cross sectional view of a substrate 32000 with multiple microfluidic open channels 32200, 32400, and 32600. It should be appreciated that the microfluidic channels can have any cross sectional geometry, such as triangular, rectangular, circular, elliptical, etc. The open microfluidic channels 32200, 32400, and 32600 are shown as having a circular or half circular cross sectional geometry. Open microfluidic channel 32200 is shown without fluid present. Open microfluidic channel 32400 is shown with fluid 33000. Fluid 33000 forms a convex meniscus. Fluid 33200 forms a concave meniscus. It should be appreciated that whether a convex or concave meniscus forms depends on the fluid. For example, a convex meniscus occurs when the particles in the liquid have a stronger attraction to each other than to the material of the container.

FIG. 16C shows a top view of a substrate with an open microfluidic channels. As shown in FIG. 16C, the substrate 34000 contains an open microfluidic channel 34200. The open microfluidic channel 34200 contains a fluid 34600 that forms a convex meniscus at the open end 34800. Due to surface tension between the fluid and the open microfluidic channel 34200, the fluid is retained within open microfluidic channel 34200.

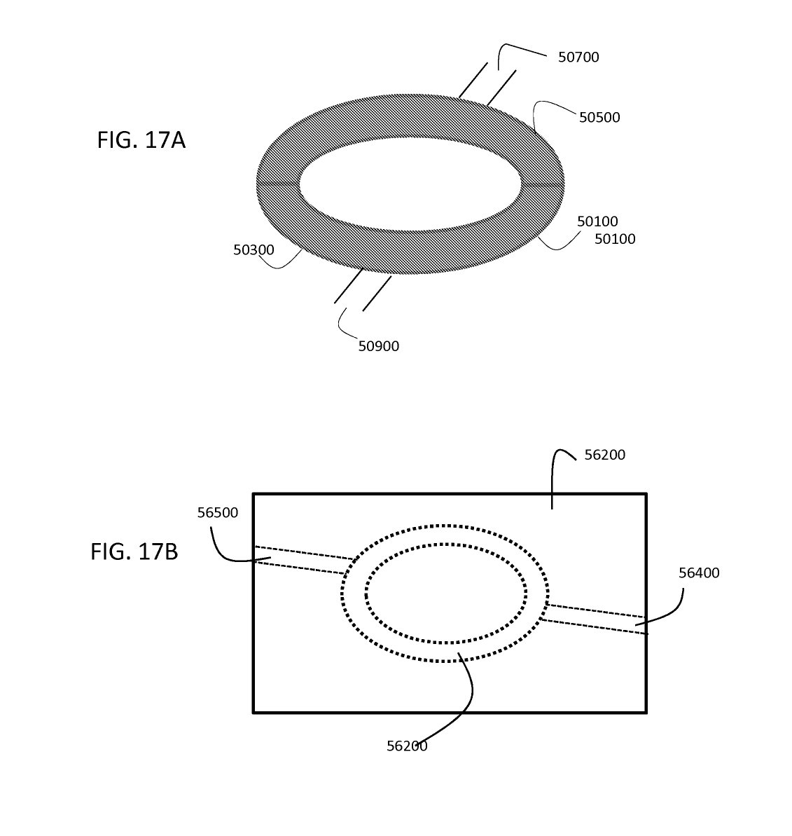

As shown in FIG. 17A, a microfluidic channel may be substantially circular 50500. Within the circular microfluidic channel 50500, fluids and droplets may be flowed, as discussed below. The microfluidic channel 50500 is configured with an input microfluidic channel 50700. Microfluidic channel 50700 may be used to introduce fluid and/or droplets into the circular microfluidic channel 50500. The circular microfluidic channel 50500 is configured with an outlet microfluidic channel 50900. Microfluidic channels 50700 and 50900 may be aligned with other microfluidic channels or with chambers of a multichannel system. Circular microfluidic channel 50500 may be configured with a rotor (not shown). FIG. 17B depicts a circular microfluidic channel 56200 that is formed within substrate 56100. Microfluidic channel 56200 may be open, closed, or a hybrid channel. Microfluidic channels 56500 and 56400 may serve as input and output channels to add or remove fluid.

### Multichannel Systems

In certain embodiments, the systems of the invention are multi-channel systems. There is no limit to the number of channels that can be included in systems of the invention, nor is there any limitation on the configuration of the channels.

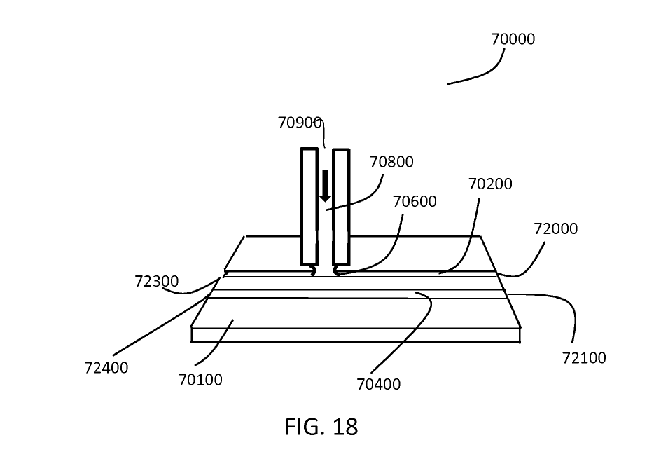

In a preferred embodiment of the invention, as shown in FIG. 18, a microfluidic system 70000 comprises a substrate 70100 which has two open microfluidic channels 70400 and 70600. Open microfluidic channel 70600 contains fluid 70200. Open microfluidic channel 70400 contains fluid 70300. Substrate 70100 and open microfluidic channels 70400 and 70600 are positioned in a substantially horizontal positon. Microfluidic system 70000 also comprises a closed microfluidic channel 70900 that contains fluid 70800, and has an open end 70500. The closed microfluidic channel 70900 is positioned over open microfluidic channel 70600, just that the center axes of the microfluidic channels are substantially orthogonal. The closed microfluidic channel 70900 is also positioned over open microfluidic channel 70600 to create an air gap between microfluidic channels 70900 and 70600, however the meniscus at open end 70500 contacts the surface of the fluid 70200 in open microfluidic channel 70600. Fluid 70800 then flows into open microfluidic channel 70600. It should be appreciated that the ends 72000 and 72300 of microfluidic channel 70600 and ends 72100 and 72400 of microfluidic channel 70400 may be open ended or sealed. In a further embodiment, microfluidic channel 70900 is taken out of alignment with microfluidic channel 70600, and aligned with open microfluidic channel 70400 in substantially the same manner microfluidic channel 70900 was aligned with microfluidic channel 70600.

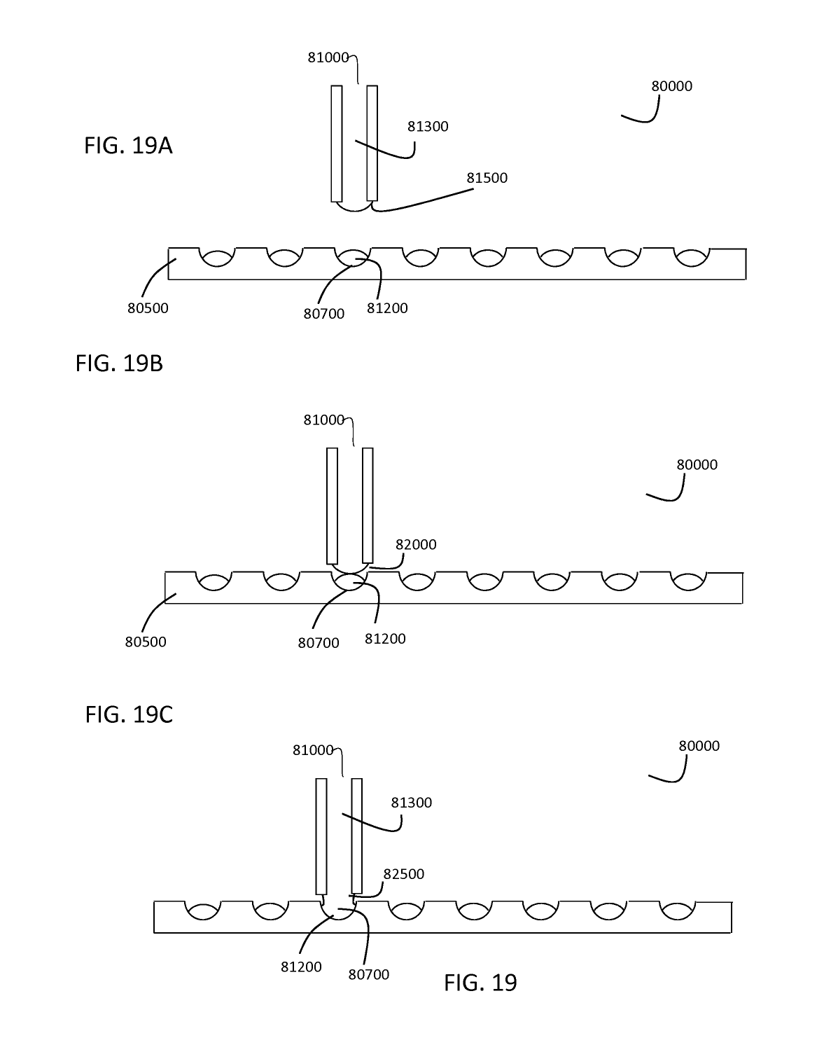

FIG. 19 provides a side view of a similar configuration shown in FIG. 18. Panels 19A -19C demonstrate the transfer of fluid from a closed microfluidic channel to an open microfluidic channel. FIGS. 19A-19C depict a microfluidic system 80000. Microfluidic system 80000 is shown with a closed microfluidic channel 81000 and a substrate 80500 that contains a plurality of open microfluidic channels 80700. Open microfluidic channels 80700 contain a fluid 81200. Closed microfluidic channel 81000 contains a fluid 81300 and has an open end 81500. As shown in FIG. 19A, the fluid 81300 is contained within microfluidic channel 81000. When closed microfluidic channel 81000 is not aligned with another microfluidic channel, whether a closed or opened microfluidic channel, the fluid is retained due to channel geometry and surface tension. As shown in FIG. 19B, microfluidic channel 81000 is positioned to align with open microfluidic channel 80700. Microfluidic channels 81000 and 80700 are aligned so that the center axes of the microfluidic channels are substantially orthogonal. Microfluidic channels 81000 and 80700 are also aligned to create an air gap 82000. As shown in FIG. 19C, the fluids 81300 and 81200 combine at air gap 82000, allowing fluid 81300 to flow into microfluidic channel 80700. The flow can be due to gravitational forces, or other forces acted on the system 80000.

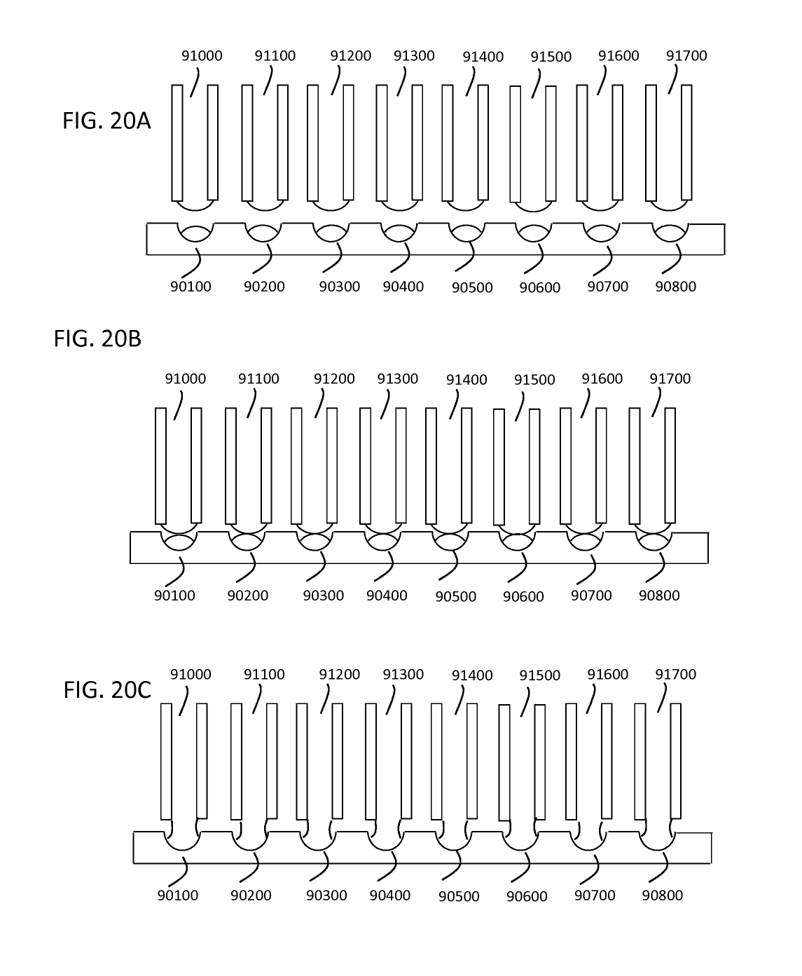

It should be appreciated that the microfluidic channels of the invention can be arranged in any configuration. Similar to the arrangement depicted in FIG. 19, FIG. 20 depicts a microfluidic system 90000 in which closed microfluidic channels 91000, 91100, 91200, 91300, 91400, 91500, 91600, and 91700 are configured to align with a series of open microfluidic channels 90100, 90200, 90300, 90400, 90500, 90600, 90700, and 90800. Any number of microfluidic channels can be arranged. The series of closed microfluidic channels contain fluids. As shown in FIG. 20A, when the closed microfluidic channels are not aligned, fluid does not flow. As shown in FIG. 20B, microfluidic channels 91000, 91100, 91200, 91300, 91400, 91500, 91600, and 91700 are positioned to align with at least one of the open microfluidic channels 90100, 90200, 90300, 90400, 90500, 90600, 90700, and 90800. When an open microfluidic channel 90100, 90200, 90300, 90400, 90500, 90600, 90700, and 90800 is aligned with a closed microfluidic channel 91000, 91100, 91200, 91300, 91400, 91500, 91600, and 91700 so that the center axes of the microfluidic channels are substantially orthogonal, and the menisci are in contact, fluid flows from the closed microfluidic channel into the open microfluidic channel. It should be appreciated that one or more microfluidic channels may align. Also, it should be appreciated, that the series of closed microfluidic channels 91000, 91100, 91200, 91300, 91400, 91500, 91600, and 91700 or the series of open microfluidic channels 90100, 90200, 90300, 90400, 90500, 90600, 90700, and 90800 can be slid to align at least one or more microfluidic channels, disengage alignment, and then align with at least one other microfluidic channels.

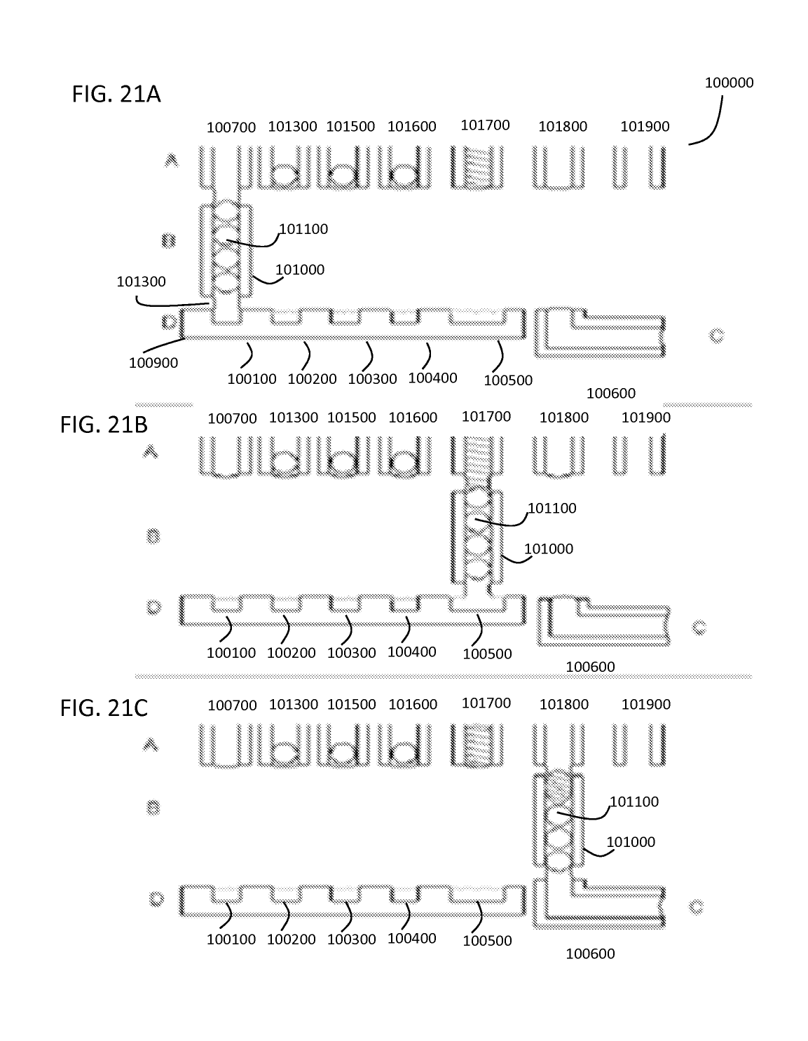

An alternative embodiment of a microfluidic system 100000 is shown in FIGS. 21A-21C. As shown in FIG. 21, a plurality of open microfluidic channels 100100, 100200, 100300, 100400, and 100500 are housed in substrate 100900. Microfluidic system 100000 also comprises a closed microfluidic channel 101000, which contains fluid 101100 and has open ends 101200 and 101300. Fluid 101100 may contain droplets, as shown in FIGS. 21A-21C. Microfluidic channel 101000 can align with any of open microfluidic channels 100100, 100200, 100300, 100400, and 100500. As shown in FIG. 21A, microfluidic channel 101000 is aligned with microfluidic channel 100700 so that fluid flows from microfluidic channel 100700, into microfluidic channel 101000, and into open microfluidic channel 100100. Microfluidic channel 101000 acts as a shuttle for fluid between channels at level A and at level D. As shown in FIG. 21B, microfluidic channel 101000 is disengaged from open microfluidic channel 100700 and aligns with microfluidic channel 101700 to allow fluid from microfluidic channel 101700 to flow into microfluidic channel 101000, and then into microfluidic channel 100500. It should be appreciated that microfluidic channel 101000 can align with any of microfluidic channels at level A and at level D. In FIG. 21C, the shuttle, or microfluidic channel 101000 is aligned with microfluidic channel 101800 and microfluidic channel 100600. With the alignment shown in FIG. 21C, fluid flows from microfluidic channel 101800 into microfluidic channel 101000 and then into microfluidic channel 100600.

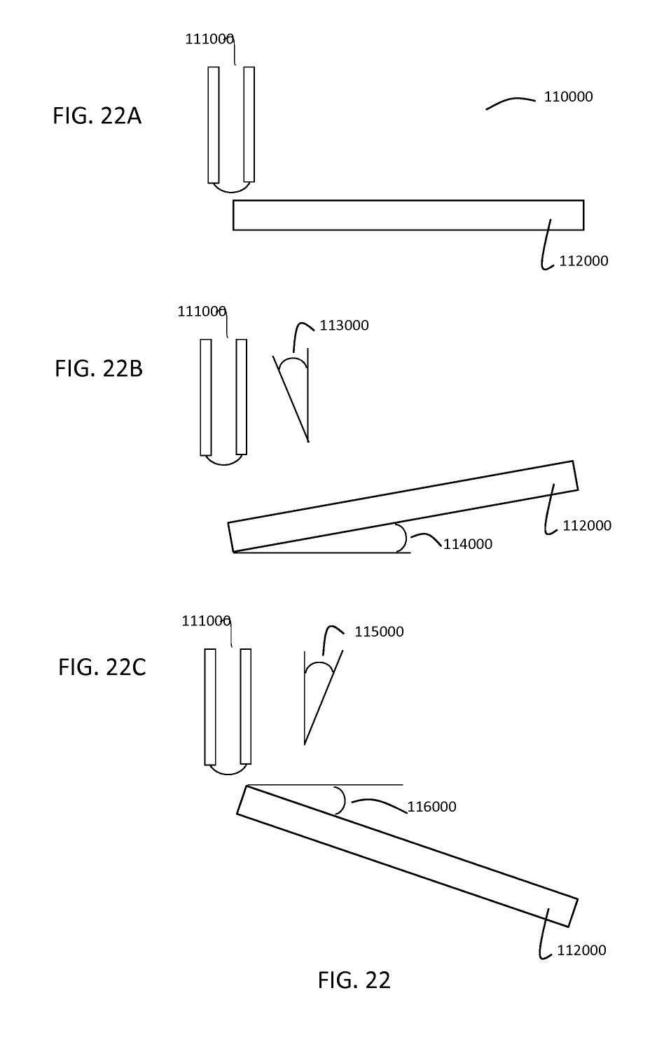

Microfluidic channels of the invention may be aligned vertically or horizontally. Microfluidic channels may be aligned substantially vertically or substantially horizontally. One microfluidic channel may be substantially vertically while another may be substantially horizontal. As shown in FIG. 22A, microfluidic channel 111000 is positioned proximate to substrate 112000. Substrate 112000 contains open microfluidic channels (not shown) or hybrid microfluidic channels (not shown). In FIG. 22A, microfluidic channel 111000 is aligned vertically while substrate 112000 is aligned horizontally. In FIG. 22B, microfluidic channel 111000 is aligned substantially vertically while substrate 112000 is aligned substantially horizontally. Microfluidic channel 111000 may be positioned at any angle 113000 from a vertical position. Angle 113000 may include any angle ranging from 90 degrees to 180 degrees, 90 degrees to 170 degrees, 90 degrees to 160 degrees, 90 degrees to 150 degrees, 90 degrees to 140 degrees, 90 degrees to 130 degrees, 90 degrees to 120 degrees, and 90 degrees to 110 degrees. Substrate 112000 may be positioned at any angle 114000 from a horizontal position. Angle 114000 may include any angle ranging from 0 degrees to 90 degrees, 0 degrees to 80 degrees, 0 degrees to 70 degrees, 0 degrees to 60 degrees, 0 degrees to 50 degrees, 0 degrees to 40 degrees, 0 degrees to 30 degrees, 0 degrees to 20 degrees, and 0 degrees to 10 degrees. As shown in FIG. 22C, substrate 112000 may positioned at an angle 116000. Angle 116000 may include any angle ranging from 270 degrees to 360 degrees, 280 degrees to 360 degrees, 290 degrees to 360 degrees, 300 degrees to 360 degrees, 310 degrees to 360 degrees, 320 degrees to 360 degrees, 330 degrees to 360 degrees, 340 degrees to 360 degrees, and 350 degrees to 360 degrees. As shown in FIG. 22C, microfluidic channel 111000 may positioned at an angle 115000. Angle 115000 may include any angle ranging from 0 degrees to 90 degrees, 10 degrees to 90 degrees, 20 degrees to 90 degrees, 30 degrees to 90 degrees, 40 degrees to 90 degrees, 50 degrees to 90 degrees, 60 degrees to 90 degrees, 70 degrees to 90 degrees, and 80 degrees to 90 degrees.

Droplet Formation by Fluid Segmentation within Movable Channels

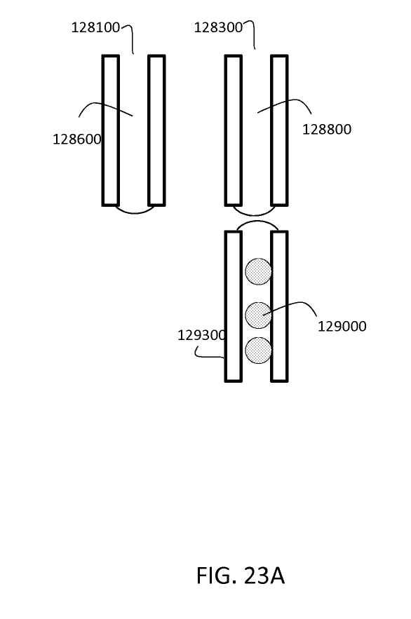

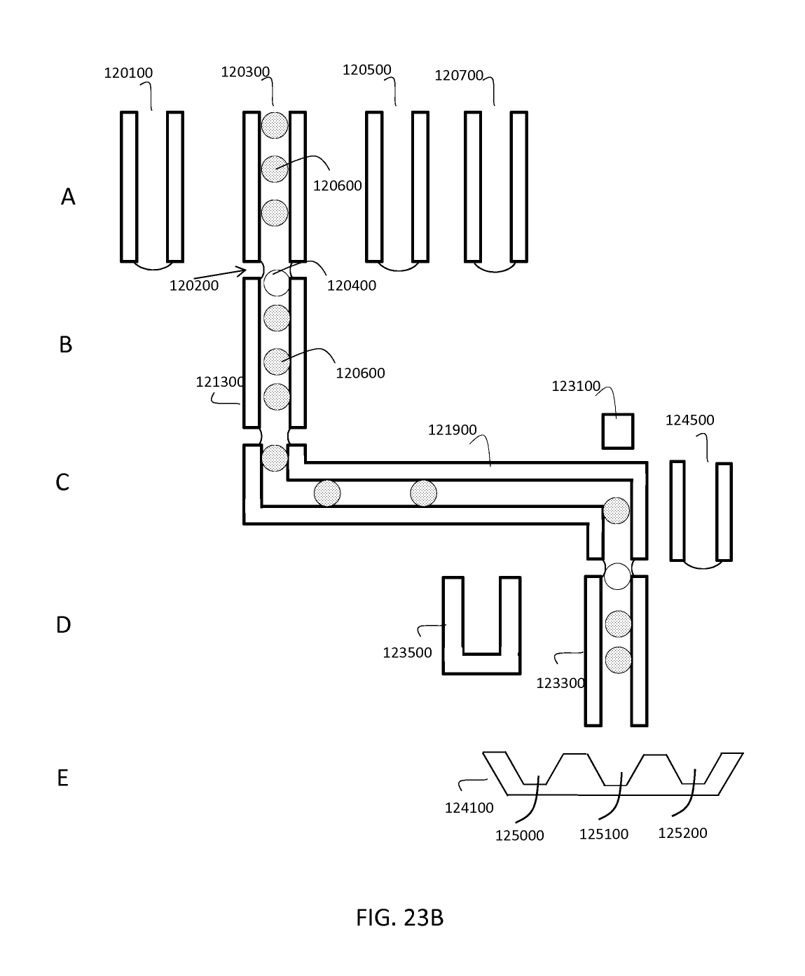

Microfluidic systems of the invention can be used to form droplets through the movement of the different channels. The microfluidic device can be used to produce single or multiple emulsions with precise control of both the contents and size of the drops. FIG. 23A depicts an arrangement of microfluidic channels for forming droplets. Microfluidic channel 128100 contains a fluid 128600 which is immiscible with aqueous fluids, such as an oil. Microfluidic channel 128300 contains an aqueous fluid 128800. To create droplets within microfluidic channel 129300, microfluidic channels aligns with microfluidic channel 128100 to form an air gap (not shown) where the fluid 128600 spans the air gap and flows into microfluidic channel 129300. As discussed herein, microfluidic channels are aligned by sliding one microfluidic channel proximate to another microfluidic channel, causing an air gap between the microfluidic channels to form. Microfluidic channel 129300 is disengaged or misaligned with microfluidic channel 128100 to allow a small volume of fluid 128600 to flow into microfluidic channel 129300. Microfluidic channel 129300 is then aligned with microfluidic channel 128300 to allow a small volume of aqueous fluid 128800 to flow into microfluidic channel 129300. Fluid 128800 is immiscible with fluid 128600 present in microfluidic channel 129300 causing droplets 129000 to form, which contains fluid 128800. Microfluidic channel 129300 can align in an alternating pattern to form droplets 129000. In the alternative, oil droplets can be formed in an aqueous phase using the technique described above.

FIG. 23B depicts an arrangement using microfluidic systems of the invention to form droplets from the fluid contained in microfluidic channel 120100 and insert the formed droplets into a stream of droplets from microfluidic channel 120300. As shown in FIG. 23B, microfluidic channel 121300 can align with several microfluidic channels: 120100, 120300, 120500, and 120700. In aspect of the invention, microfluidic channel 121300 can be slid or moved in order to align with microfluidic channels 120100, 120300, 120500, and 120700. Microfluidic channels 120100, 120300, 120500, and 120700 may be stationary or moveable. Furthermore, microfluidic channels 120100, 120300, 120500, and 120700 may be moved together, or may be moved independently, depending on the arrangement of substrates employed. Microfluidic channel 120100 contains a fluid. As shown in FIG. 23B, microfluidic channel 120300 contains droplets 120600 within an immiscible fluid.

As shown in FIG. 23B, microfluidic channel 121300 contains droplets 120600 and 120400. Droplet 120400 contains the fluid from microfluidic channel 120100, and the fluid in microfluidic channel 120100 is immiscible with the fluid in microfluidic channel 121300. Microfluidic channel 121300 was aligned with microfluidic channel 120100 for a span of time to only allow for a small volume of fluid to pass from microfluidic channel 120100 into microfluidic channel 121300. This small volume of fluid formed into a droplet 120400 within microfluidic channel 121300, which contains a fluid immiscible with the droplets in microfluidic channel 120300 and the fluid in microfluidic channel 120100. Alignment may occur to allow for a small volume of fluid to pass between microfluidic channels to generate a droplet in the receiving microfluidic channel. As shown in FIG. 23B, microfluidic channel 121300 is aligned with microfluidic channel 120300 so that a portion of fluid spans an air gap 120200 to thereby flow fluid and droplets 120600 from microfluidic channel 120300 into microfluidic channel 121300. Microfluidic channel 121300 may slide or move to align with microfluidic channels 120100, 120300, 120500, and 120700. Microfluidic channel 121300 can be slid to align with any of the microfluidic channels, or microfluidic channel 121300 can be positioned so as not to align with any microfluidic channel, and therefore does not receive fluid or droplets. Microfluidic channel 121300 may align with microfluidic channel 121900. Microfluidic channel 121900 may be movable or may be stationary. Alignment between microfluidic channel 121300 and microfluidic channel 121900 allows for flow between the two channels.

As shown in FIG. 23B, microfluidic channel 123300 is aligned with microfluidic channel 121900 to allow fluid to flow between the two microfluidic channels. Microfluidic channel 123300 can be slid to disengage from microfluidic channel 121900. A chamber 123500 can be slid to engage with microfluidic channel 121900 to thereby allow fluid to flow from microfluidic channel 121900 into chamber 123500. Chamber 123500 can be a waste chamber. As shown in FIG. 23B, microfluidic channel 123300 can align with wells in a well plate 124100 to deliver fluid from microfluidic channel 123300 to the wells in the well plate 124100. Alternatively, 124100 can be a substrate comprising at least one open microfluidic channel or one hybrid channel. Open microfluidic channels or wells 125000, 125100, and 125200 may contain fluid (not shown).

As shown in FIG. 23B, the droplets are flowed through numerous levels or layers, A, B, C, D, and E. For example, microfluidic channels 120100, 120300, 120500, and 120700 are at layer A. Microfluidic channel 121300 is at layer B. Microfluidic channels 121900 and 124500 are at layer C. Microfluidic channel 123300 and waste container 123500 are at layer D. Well plate 124100 is at layer E. It should be appreciated that at each level, the microfluidic channels, waste containers, or other components can move in any direction to align with other components. For example, at level D, microfluidic channel 123300 can move to align with microfluidic channel 121900 or to be disengaged from microfluidic channel 123300. In addition, waste container 123500 can move and align with microfluidic channel 121900, or can be disengaged from microfluidic channel 121900. Although FIG. 23B shows only a few layers (A-E) of microfluidic channels, a plurality of layers can be joined, arranged or aligned to carry out various assays and processes. Detectors can be placed at each level or layer to screen for alterations or detectable signals from the droplets or fluids. For example, a detector can be placed at any level or layer to screen for cells within the droplets that have died and should therefore be diverted to a waste container. In this embodiment, the dead cells are eliminated from the assay, and are not passed to another level or layer for processing.

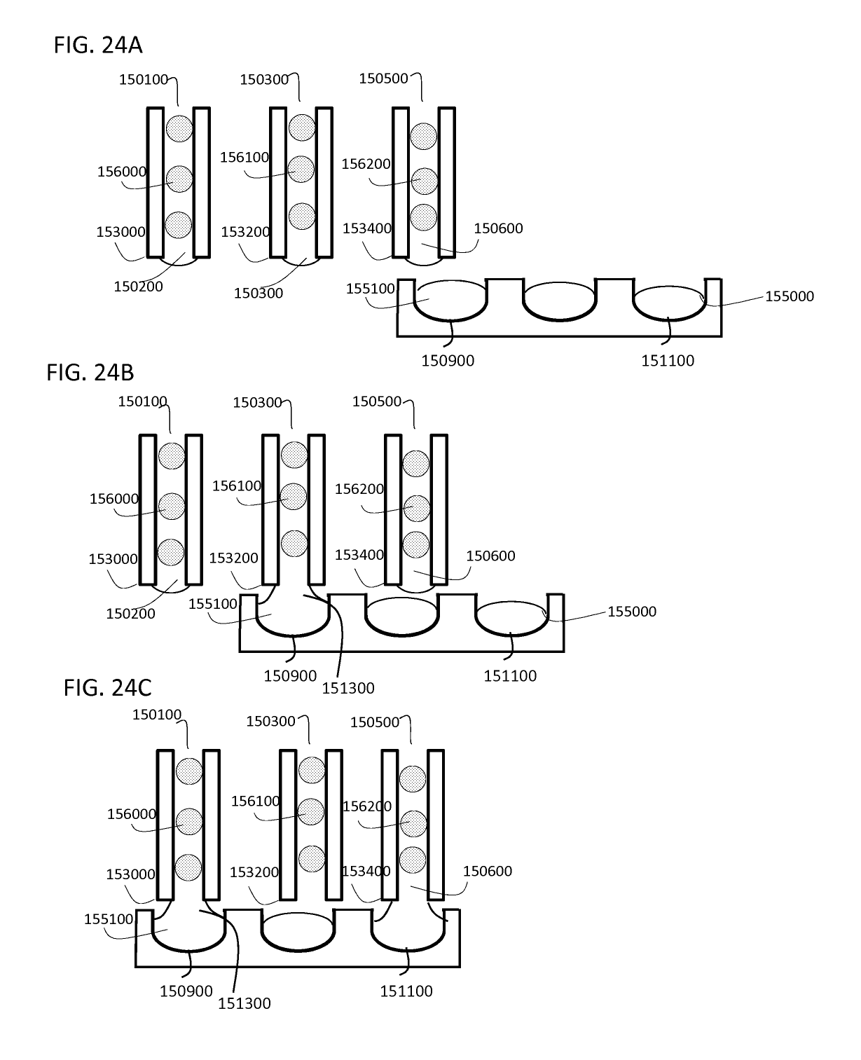

In an alternative embodiment, FIGS. 24A-24C depict a multi-channel system configured to allow microfluidic channels to slide or move relative to each other to alter alignment of the channels. FIG. 24A shows multiple microfluidic channels 150100, 150300, and 150500 which are open at ends 153000, 153200, and 153400. Microfluidic channels 150100, 150300, and 150500 each may contain a fluid; for example microfluidic channel 150100 contains fluid 150200. Fluids 150200, 150300, and 150600 may be composed of the same components or may be composed of differing components. Each fluid 150200, 150300, and 150600 is retained in the microfluidic channel by forces such as surface tension. As discussed above, an aspect of the invention is that fluid does not flow from the microfluidic channel unless aligned with another microfluidic channel. Also, each microfluidic channel 150100, 150300, and 150500 contains droplets 156000, 156100, and 156200. As discussed above, droplets may be composed of various fluids and components. Additionally, the droplets 150100, 150300, and 150500 may comprise the same materials and components, or they droplets may comprise differing materials and components. Additionally, microfluidic channels 150100, 150300, and 150500 may be slidable or moveable together or independent of one another. FIG. 24A also depicts open microfluidic channels 150900 and 151100. It should be appreciated that microfluidic channels 150900 and 151100 are shown in FIG. 24A to contain fluids 155000 and 155100. However, it should also be appreciated that microfluidic channels 150900 and 151100 may not contain fluids. Microfluidic channels 150900 and 151100 may be moved independent of one another or may be moved together. It is an aspect of the invention that microfluidic channels may be arranged in any configuration and manner. As shown in FIG. 24A, open microfluidic channels 150900 and 151100 are positioned to be disengaged from microfluidic channels 150100, 150300, and 150500. In this positioning of the microfluidic channels, microfluidic channels 150100, 150300, and 150500 are prevented from flowing fluid due to the physical properties of the microfluidic channel and the immiscible fluid, e.g. surface tension, as discussed above.

Open microfluidic channels 150900 and 151100 may be slid or moved to align with any of the microfluidic channels 150100, 150300, or 150500. FIG. 24B depicts open microfluidic channels 150900 and 151100 that have been moved or slid relative to microfluidic channels 150100, 150300, or 150500. As shown in FIG. 24B, microfluidic channel 150900 has been slid to engage at least one of microfluidic channels 150100, 150300, and 150500. It should be appreciated that the moving or sliding of microfluidic channel 150900 or 151100 may involve movement in any plane or direction. In FIG. 24B, microfluidic channel 150300 is aligned with open microfluidic channel 150900. The alignment may cause an air gap 151300. Also, as discussed above, it is not necessary for the microfluidic channels to be aligned so that the microfluidic channels are flush. Rather, an air gap 151300 may be present between the two microfluidic channels. The alignment of microfluidic channels 150300 and 150900 forms an air gap at 151300. The formation of the air gap 151300 results in a portion of fluid spanning air gap 151300 and allows for fluid 150300 and droplets 156100 to flow from microfluidic channel 150300 into microfluidic channel 150900. In this positioning, open microfluidic channel 150900 receives fluid 150300 and droplets 156100 from microfluidic channel 150300.

The microfluidic channels of the invention may be slid or move in several iterations. For example, as shown in FIG. 24C, open microfluidic channel 150900 has been slid to align with microfluidic channel 150100. As discussed previously, microfluidic channel 150900 was aligned with microfluidic channel 150300 and received fluid 150300 and droplets 156100. Open microfluidic channel 150900 now contains fluid 150300 and fluid 150200 and droplets 156100 and 1560. In this embodiment, the components of the two different microfluidic channels are mixed. Additionally, as shown in FIG. 24C, open microfluidic channel 151100 is aligned with microfluidic channel 150500. Microfluidic channels 150900 and 151100 can be slid or moved at the same time, or independently of each other, depending on the configuration of open microfluidic channels 150900 and 151100 and their respective substrates.

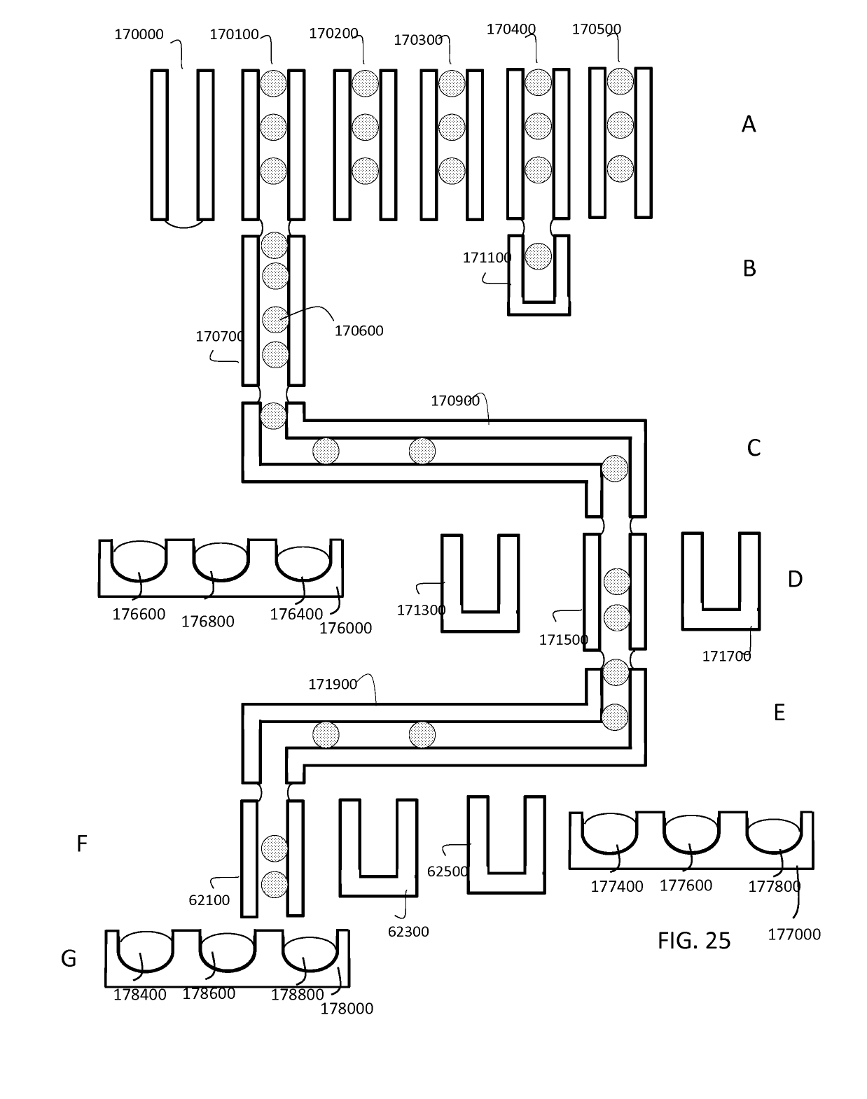

FIG. 25 depicts another embodiment of the invention. As shown in FIG. 25, a plurality of microfluidic channels, 170000, 170100, 170200, 170300, 170400, and 170500 contain carrier fluid and droplets. Microfluidic channel 170100 is aligned with microfluidic channels 170700 to allow fluid and droplets to flow from microfluidic channels 170100 to microfluidic channels 170700 and then to microfluidic channels 170900. Microfluidic channel 170300 is aligned with microfluidic channels 171100, which can serve as a microfluidic channels and as a chamber. The chamber can be a waste chamber, a collection chamber, or other purpose chamber employed with the device and methods of the invention. As shown in FIG. 25, microfluidic channels 171100 accepts fluid from microfluidic channels 170300, and microfluidic channels 171100 serves as a waste chamber. Microfluidic channels 170000, 170100, 170200, 170300, 170400, and 170500 may be attached to or positioned within a surface, wherein the surface is movable. It should be appreciated that microfluidic channels 170000, 170100, 170200, 170300, 170400, and 170500 may be moved together or independently of one another. Microfluidic channel 170700 also may be slid or remain in a stationary position. Microfluidic channel 171100 may be slid or remain in a stationary position. Fluid and droplets flow into microfluidic channel 170900 when a microfluidic channel aligns with microfluidic channel 170900. Microfluidic channel 170900 may be slidable or stationary. Fluid and droplets can flow from microfluidic channel 170900 to microfluidic channel 171900 when microfluidic channel 171500 is aligned therebetween. Microfluidic channel 171300 can align with microfluidic channel 170900 to divert the fluid and/or droplets to a waste chamber. Microfluidic channel 171700 can align with microfluidic channel 171700 to diver the fluid and/or droplets to a collection chamber. Substrate 1760 containing open microfluidic channels 176400, 176600, and 176800 can align with microfluidic channel 170900 to flow fluid and/or droplets into any one of open microfluidic channels 176400, 176600, and 176800. Substrate 177000 containing open microfluidic channels 177400, 177600, and 177800 can also align with microfluidic channel 171900. Microfluidic channels 172100, 172300, and 172500 can align with microfluidic channel 171900 to divert or direct the flow of droplets into another microfluidic channel, a waste chamber or a collection chamber. Alternatively, droplets can be diverted into an incubation chamber for further processing. Microfluidic channel 172100 can align and deliver fluid and/or droplets in substrate 178000 containing open microfluidic channels 178400, 178600, and 178800.

As shown in FIG. 25, several levels or layers, A-G are depicted with differing components at each level. At layer A, a plurality of microfluidic channels 170000-170500 contain various fluids. The fluids may contain droplets, nucleic acids, drug molecules, etc., depending on the assay. Layer B contains a microfluidic channel 170700 which acts as a shuttle for fluids between layers A and C. Layer B also contains a waste compartment for collecting waste, such as dead cells, from layer A. Layer C contains a substantially horizontal microfluidic channel 170900 which may be open or closed. Layer D contains various components, such as a compartment 171300 waste compartment 171700, a shuttle 171500 and a substrate 176000 which contains a plurality of open channels. FIG. 25 also depicts levels E, F, and G, which also contain various layers, as described above. It should be appreciated that the architecture of a system can be varied, containing any number of components at each layer or level. A system can be composed of one or more layers, and each layer may contain any variety of components.

### Circulating Channel

As discussed above, microfluidic channels may be nonlinear, or even contain branches. In some embodiments, microfluidic channels of the present invention may be substantially circular. See FIGS. 17A and 17B. In some embodiment of the invention, the circulating channel is used so that cell culturing can be performed in systems of the invention. Cells are held in microfluidic aqueous droplets that are separated from one another by silicone oil, or any immiscible fluid. These droplets are then introduced into the circular microfluidic channel. The droplets are held in a circular cross-section channel which has a rotating inner wall and a stationary outer wall. Rotation is achieved by a rotor or similar device. In that manner, the droplets can circulate for as long as is required, for example for culturing of a cell in the droplet. Waste material from the cells diffuses to the lower portion of the circular path. An outlet channel configured at the lower portion of the circular path removes the waste material. An inlet channel configured to deliver fluids into the circular path allows for the introduction of additional fluids, such as cell medium.

In a preferred embodiment of the invention, cells are introduced into a circular channel and flowed in the circular path. Waste from the cells diffuses out of the droplets into the immiscible fluid. The waste from the cells is removed and fluids are replenished to approximately maintain the volume in the circular path. For example, if the droplets contain cells and cell medium, the cell medium may be replenished by coalescing with a droplet containing cell medium.

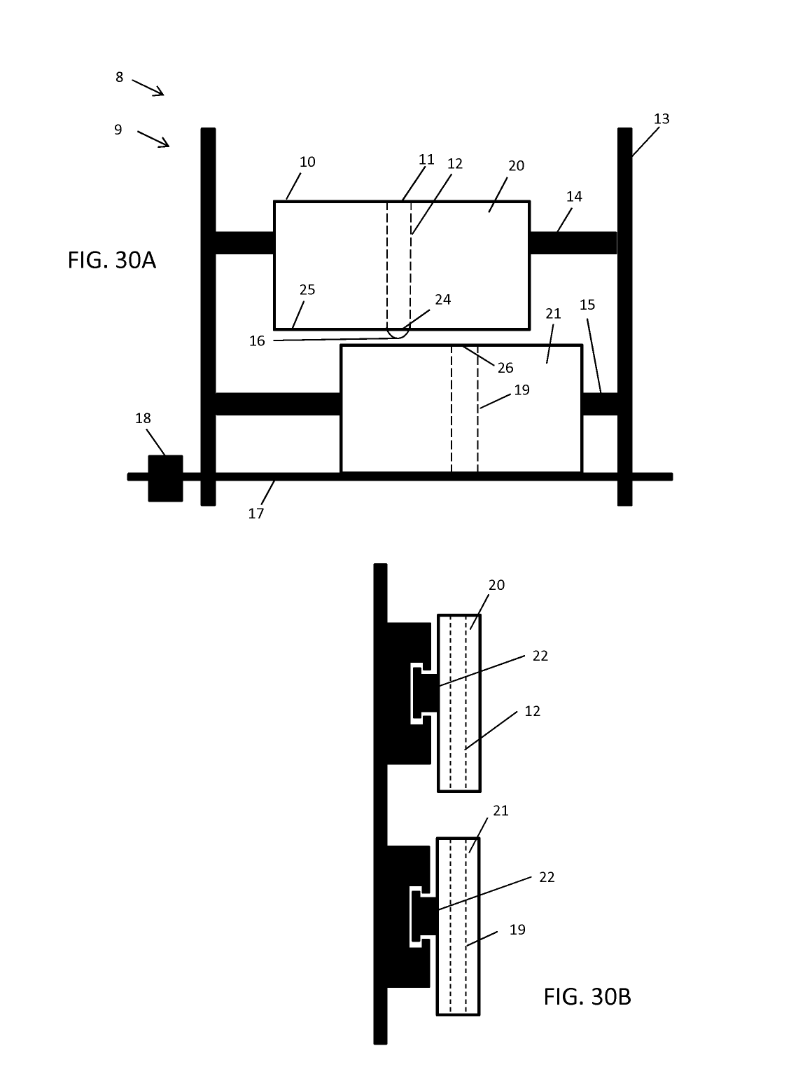

In some embodiments of the invention, as shown in FIGS. 30A and 30B, a microfluidic system 8 comprises a first substrate 20 with at least one reservoir 12 in fluid communication with an upper opening 11 at an upper surface 10 of the first substrate 20 and a lower opening 24 at a lower surface 25 of the first substrate 20, wherein the lower opening 24 is dimensioned such that a liquid 16 held in the reservoir 12 is prevented from flowing through the lower opening 24 and into ambient atmosphere by surface tension. The microfluidic system 8 also comprises a mechanical subsystem 9 supporting the first substrate 20 in an orientation such that liquid dispensed to the upper opening 11 flows into the reservoir 12. The mechanical subsystem 9 comprises an upper rail 14 and a lower rail 15. The microfluidic system 8 also comprises a second substrate 21 coupled to 22 and controlled by the mechanical subsystem 9, the second substrate 21 comprising a receptacle 19 open to a surface 26 of the second substrate 21, wherein operation of the mechanical subsystem 9 while a liquid 16 is held within the reservoir 12 brings the second substrate 21 into contact with a surface of the liquid 16, causing the liquid 16 to flow into the receptacle 19.

In some embodiments the systems are coupled or operably linked to a mechanical subsystem 9 comprising a chassis 13. A substrate of the invention may be operably coupled to a drive rail 17 which is operably connected to a motor 18, such that operation of the motor causes movement of the drive rail 17 which slides a substrate 21 which is coupled to a rail 15 by an attachment configuration 22.

### Robotic Stages

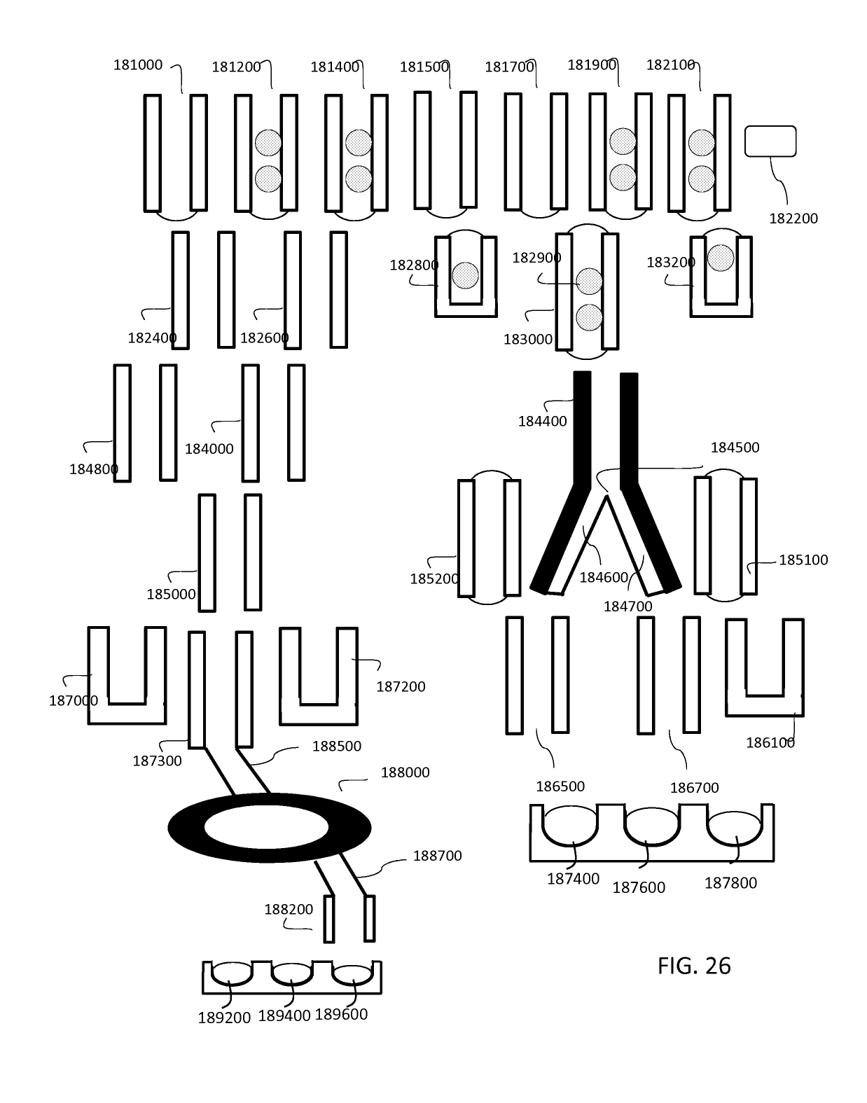

The systems of the invention can be controlled by robotic stages. As discussed above, the substrates of the invention may be coupled to mechanical subsystems. As seen in FIGS. 25 and 26, various components comprise the microfluidic devices of the invention. It should be appreciated that the various components can be controlled by robotics. In an exemplary system of the invention, the microfluidic components are controlled by robotics, thereby allowing for assays to be further controlled through alignment and misalignment of the components. A system may include a data processor, a motion controller, a robotic arm assembly, a monitor element, a central processing unit, a microliter plate of source material, a stage housing, a robotic arm, a stage, a pressure controller, a conduit, a mounting assembly, a pin assembly, and microfluidic component elements. Stages and arms of the robotic assembly are moveable in the x-y-z planes.

The data processor can be a conventional digital data processing system such as an IBM PC compatible computer system that is suitable for processing data and for executing program instructions that will provide information for controlling the movement and operation of the robotic assembly. It will be apparent to one skilled in the art that the data processor unit can be any type of system suitable for processing a program of instruction signals that will operate the robotic assembly that is integrated into the robotic housing. Optionally the data processor can be a micro-controlled assembly that is integrated into the robotic housing. In further alternative embodiments, the system need not be programmable and can be a single board computer having a firmware memory for storing instructions for operating the robotic assembly.

The controller can be electronically coupled between the data processor and the robotic assembly. The controller is a motion controller that drives the motor elements of the robotic assembly for positioning the robotic arm at a selected location. Additionally, the controller can provide instructions to the robotic assembly to direct the pressure controller to control the volume of fluid or gas injected into the system. It should be appreciated that any design of a robotic assembly could be used in the present invention. The design and construction of robotic assemblies are well known in the art of electrical engineering, and any controller element suitable for driving the robotic assembly can be used. Accordingly, it will be apparent to one of skill in the art that alternative robotic systems can be used.

The devices and methods of the present invention have utility in the area of cell culturing. A multichannel device of the invention can be employed to culture cells in a controlled and stable environment. For example, FIG. 25 depicts a multichannel device for manipulating and directing droplets. As shown in FIG. 25, microfluidic channels 170100, 170200, 170300, 170400, and 170500 contain droplets. In a cell culture assay, the droplets 170600 contain cells and cell medium to ensure the health and proliferation of the cells. Microfluidic channel 170000 contain cell medium. To add cell medium to the droplets, microfluidic channel 170700 aligns with microfluidic channel 170000 for a span of time to create a droplet of cell medium. The cell medium droplet merges with a cell droplet 170600, as discussed above. Microfluidic channel 170700 aligns with microfluidic channel 170900 to allow the droplets containing cells and cell medium to flow into microfluidic channel 170900. Droplets and/or fluids in microfluidic channel 170900 can be flowed into open microfluidic channels 176600, 176800, or 176400. Substrate 176000 can be moved to align with microfluidic channel 170900, or microfluidic channel 170900 can be moved to align with open microfluidic channels 176400, 176600 or 176800. It should be appreciated that waste chamber 171100 may align with microfluidic channels 170100, 170200, 170300, 170400, and 170500 to remove droplets containing non-viable cells. It should also be appreciated that chamber 171700 may align with microfluidic channel 170900 to collect the droplets containing cells for detection, assay, or incubation. Droplets that are not diverted flow from microfluidic channel 170900 into microfluidic channel 171900 when microfluidic channel 171500 is positioned there between. It should also be appreciated that chambers 172300 and 172500 can align with microfluidic channel 171900 to divert droplets into chambers 172300 and 172500. It should be appreciated that chambers 172300 and 172500 may be to collect droplets containing non-viable cells or to incubate the cell containing droplets. Substrate 178000 containing open microfluidic channels 178400, 178600, and 178800 can align with microfluidic channel 172100 to allow flow of fluids and/or droplets there between.

In an alternative embodiment, droplets containing cells may be merged or combined with compounds for investigation of reactivity and efficacy. For example, FIG. 25 depicts a multichannel device for manipulating and directing droplets. As shown in FIG. 25, microfluidic channels 170100, 170200, 170300, 170400, and 170500 contain droplets. In a cell investigation assay, the droplets 170600 contain cells and cell medium to ensure the health and proliferation of the cells. Microfluidic channel 170000 contains a fluid comprising a test compound. It should be appreciated that any test compound may be used in the assay. To add the target compound to the droplets, microfluidic channel 170700 aligns with microfluidic channel 170000 for a span of time to create a droplet containing the target compound. The target compound droplet merges with a cell droplet 170600, as discussed above. Microfluidic channel 170700 aligns with microfluidic channel 170900 to allow the droplets containing cells and target compound to flow into microfluidic channel 170900. It should be appreciated that waste chamber 171100 may align with microfluidic channels 170100, 170200, 170300, 170400, and 170500 to remove droplets containing non-viable cells. It should also be appreciated that chamber 171700 may align with microfluidic channel 170900 to collect the droplets containing cells for detection, assay, or incubation. Droplets that are not diverted flow from microfluidic channel 170900 into microfluidic channel 171900 when microfluidic channel 171500 is positioned there between. It should also be appreciated that chambers 172300 and 172500 can align with microfluidic channel 171900 to divert droplets into chambers 172300 and 172500. It should be appreciated that chambers 172300 and 172500 may be to collect droplets containing non-viable cells or to incubate the cell containing droplets. In the other preferred embodiments, substrates 176000, 177000, and/or 178000 can align to receive fluids and/or droplets.

A multichannel system is depicted in FIG. 26. In regards to FIG. 26, the microfluidic channels and chambers may be aligned by sliding the microfluidic channels proximately to one another. Microfluidic channels may be slid together or independently of one another, as discussed above. Chambers may also be slid in together, or independently. Microfluidic channels that are slid together may be located on the same substrate, or may be located on different substrates. Similar arrangement can be with chambers. As discussed above, alignment may create air gaps (not shown). The liquid in the channels or chambers bridge the air gap to allow fluid and/or droplets to flow from a microfluidic channel to another microfluidic channel, or from a microfluidic channel to a chamber. As shown in FIG. 26, multiple microfluidic channels 181000, 181200, 181400, 181500, 181700, 181900, and 182100 contain droplets, fluids, or fluids containing target compounds. A detector 182200 is positioned proximate to microfluidic channel 182100. A detector can be positioned proximate to any microfluidic channel in the multichannel device. In an aspect of the invention, cells may be combined with target compounds to test or analyze the effects on cells. As depicted in FIG. 26, chamber 182800 receives a droplet from any of microfluidic channels 181000, 181200, 181400, 181500, 181700, 181900, or 182100. Chamber 182800 can be for waste or for incubating the droplet for a span of time. Similarly, chamber 183200 can receive a droplet from any of microfluidic channels 181000, 181200, 181400, 181500, 181700, 181900, or 182100. Chamber 183200 can be for waste, or to incubate the cells for any span of time. A heating element or heating source, not shown, may be proximately located to either chambers 182800 and 183200. As shown in FIG. 26, microfluidic channel 183000 received a droplet 182900 from any of microfluidic channels 181000, 181200, 181400, 181500, 181700, 181900, or 182100. Droplet 182900 passes through microfluidic channel 183000 and is then passed to microfluidic channel 184400. Microfluidic channel 184400 contains a branch and an obstruction at 184500. As stated above, the obstruction 184500 caused cells to be split. A droplet introduced into microfluidic channel 184400 would be split at obstruction 184500, causing part of the droplet to be directed down microfluidic channel 184600 and the other part of the droplet is directed down microfluidic channel 184700. Microfluidic channels 185200 and 185100 may contain fluid which contains cell medium, sample fluid, reactants, target compounds, etc. Microfluidic channel 186500 can align with microfluidic channel 184600 to receive a droplet from microfluidic channel 184600. Microfluidic channel 186500 can align with microfluidic channel 185200 to accept fluid, and can form a droplet from the fluid in microfluidic channel 185200. As discussed earlier, a droplet from microfluidic channel 184600 and a formed droplet from microfluidic channel 185200 can merge. This technique allows for the fluid from microfluidic channel 185200 to be combined with droplets from 184600. Alternatively, droplets from microfluidic channel 184600 can pass to microfluidic channel 186500 and not be merge with any other fluid. From microfluidic channel 186500, droplets can be further processed. Similarly, droplets from microfluidic channel 184700 can flow into microfluidic channel 186700 by the alignment between microfluidic channels 184700 and 186700. Either microfluidic channel 186500 and 186700 can align with any open microfluidic channel 187400, 187600, and 187800. Microfluidic channel 186700 can align with microfluidic channel 185100 to accept fluid, by forming a droplet, and can causing coalescing between a droplet from microfluidic channel 184700 and microfluidic channel 186700. Chamber 186100 can align with microfluidic channels 184600 or 184700 to collect droplets as waste or to incubate the droplets.

As shown in FIG. 26, microfluidic channels 182400 and 182600 can receive fluids and/or droplets from any of microfluidic channels 181000, 181200, 181400, 181500, 181700, 181900, or 182100. For example, microfluidic channel 182400 can align with microfluidic channel 181200 to receive a droplet. Microfluidic channel 182400 can then align with microfluidic channel 181000 to form a droplet from the fluid in microfluidic channel 181000. The fluid can contain reactants, cell medium, or target compounds. In microfluidic channel 182400, droplets from microfluidic channels 181000 and 181200 can coalesce. Similarly, microfluidic channel 182600 can receive droplets and fluids from any of microfluidic channels 181000, 181200, 181400, 181500, 181700, 181900, or 182100. For example, microfluidic channel 182600 can receive droplets from microfluidic channel 181400 and can form droplets from the fluid contained in microfluidic channel 181500. The droplets from microfluidic channels 181400 and 181500 can be coalesced by techniques and methods discussed above. By way of example, microfluidic channel may contain target compounds that are combined with droplets in microfluidic channel 181200. Microfluidic channel 184000 can align with microfluidic channels 182400 and 182600 to receive the droplets contained within microfluidic channels 182400 and 182600. Microfluidic channel 185000 can align with microfluidic channels 184000 or 184800. Microfluidic channel 184800 can contain fluid, which contains reactants, cell medium, or target compounds. By aligning with microfluidic channel 184800, microfluidic channel 185000 can create droplets from the fluid contained within microfluidic channel 184800. Droplets from microfluidic channel 184000 and 184800 can be coalesced in microfluidic channel 185000 by the methods and techniques discussed above. Droplets in microfluidic channel 185000 can be diverted into microfluidic channel 187300 by aligning microfluidic channels 185000 and 187300. Droplets in microfluidic channel 185000 can be diverted to chamber 187000, which can be waste chamber. Droplets in microfluidic channel 185000 can be diverted to chamber 187200, which can be an incubation chamber. Droplets in microfluidic channel 187300 are diverted into circular microfluidic channel 188000. Droplets may circulate via a rotor, not shown. Droplets may exit circular microfluidic channel 188000 via outlet 188700 and into microfluidic channel 188200. Microfluidic channel 188200 can align with any open channel 189200, 189400, or 189600.

For example, in a preferred embodiment, cells are encased in the droplets contained in microfluidic channels 181900 and 182100. Microfluidic channel 183000 aligns with microfluidic channel 181900 to receive droplets. The droplets are flowed into microfluidic channel 184400 to split the droplets, and thereby split the cells by aligning microfluidic channels 184400 and 183000. Droplets, and thereby cells, are split and flow into microfluidic channels 184600 and 184700. Microfluidic channel 186500 aligns with microfluidic channel 184600 and then microfluidic channel 186500 aligns with microfluidic channel 185200 to cause droplets from 184600 and 185200 to be closely positioned to allow for passive merging. The fluid in microfluidic channel 185200 is cell medium, so that passive merging causes replenishing of cell medium to the droplets. The same procedure is repeated for the droplets in microfluidic channel 184700. Following splitting and replenishing of cell medium, the droplets are incubated in a chamber (not shown) by flowing the droplets from microfluidic channels 186500 and 186700 into a chamber.

For example, in another preferred embodiment, droplets in microfluidic channel 181200 contain cells, and microfluidic channel 181000 contains a fluid containing a testing compound, (e.g., a drug molecule). Microfluidic channel 182400 aligns with microfluidic channel 181200 to receive a droplet and aligns with microfluidic channel 181000 to flow a small volume of the fluid in microfluidic channel 182400. The droplet and small volume are positioned closely to cause passive merging, as discussed above. Microfluidic channel 184800 aligns with microfluidic channel 182400 to receive a coalesced droplet and aligns with microfluidic channel 182600 to receive a small volume of the fluid contained in microfluidic channel 182600. The fluid in microfluidic channel may be cell medium, reactants, nutrients, buffer, etc. Microfluidic channel 185000 aligns with microfluidic channel 184800 to direct the droplets to circular microfluidic channel 1080 via microfluidic channel 187300. The droplets are circulated for a period of time and then diverted to microfluidic channel 188700. Open microfluidic channels 189200, 189400, and 189600, can be positioned to align with microfluidic channel 188200 to accept fluid and/or droplets. Droplets are then flowed into a chamber (not shown) for detection.

FIGS. 27A-27D depict several alternative embodiments of the open channels. FIG. 27A depicts a channel 200300 with ends 200600 and 200500. Ends 200600 and 200500 are vertical, or are configured to align with a vertical microfluidic channel. It should be appreciated that ends 2006 and 2005, or any vertical microfluidic channel, need not be perfectly vertical; to not have a slope. A vertical microfluidic channel or portion of a microfluidic channel can have a substantially vertical orientation, or can have a slope. Alignment only needs to cause fluid to flow there between.

FIG. 27B depicts an open microfluidic channel 2010 in substrate 2012. End 2014 can be opened or sealed. End 2016 is open and will flow fluid when aligned with another microfluidic channel. FIG. 27C depicts microfluidic channel 201800 in substrate 201900 with openings 202000. It should be appreciated that an open microfluidic channel can have any number of openings 202000, i.e. 1, 2, 3, etc. A microfluidic channel can align with any opening to cause fluid to flow there between. As shown in FIG. 27D, microfluidic channel 203000 in substrate 203100 is an open microfluidic channel. When channel 203000 aligns with microfluidic channel 204000 in substrate 204100, fluid flows there between. When channel 203000 is not aligned with microfluidic channel 204000, fluid does not flow there between. When microfluidic channel 204000 aligns with microfluidic channel 205000 in substrate 205100, fluid flows there between. Channels 203000, 204000, and 205000 can be open channels, closed channel, hybrid channels or any combination thereof. Microfluidic channel 205000 also contains openings 205500 which can align with microfluidic channels to cause fluid to flow there between.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1-5. (canceled)

6. A microfluidic system, the system comprising:
a first channel comprising an open end; and
a second channel comprising an open end, wherein the first and second channels are slidable relative to each other such that when the open end of the first channel and the open end of the second channel are aligned with each other, fluid flows from the first channel into the second channel.

7. The microfluidic system according to claim 6, wherein the channels are horizontally slideable relative to each other.

8. The microfluidic system according to claim 7, wherein the open end of the first channel and the open end of the second channel are exposed to atmospheric pressure.

9. The microfluidic system according to claim 8, wherein the first and second channels are arranged in relation to each such that an air gap exists when the open end of the first channel and the open end of the second channel are aligned with each other and fluid from the first channel bridges the air gap and enters the second channel.

10-11. (canceled)

12. The microfluidic system according to claim 6, wherein the system further comprises at least one collection vessel downstream of the second channel.

13. The microfluidic system according to claim 6, further comprising a third channel downstream of the second channel, wherein the third channel comprises an open end and the second and third channels are slidable relative to each other.

14-25. (canceled)

26. A method of transferring liquid within a microfluidic system, the method comprising:
holding a liquid within an open-ended microfluidic channel in a substrate by surface tension such that a surface of the liquid at a lower end of the open-ended microfluidic channel is exposed to an atmosphere around the substrate; and
bringing a second substrate into proximity with the substrate such that at least a portion of an edge of a second channel makes contact with the surface, thereby breaking the surface tension that holds the liquid within the open-ended microfluidic channel and causing the liquid to flow into the second channel.

27. The method of claim 26, wherein the lower end defines an opening no greater than 0.6 mm across.

28. The method of claim 26, wherein the second substrate is mechanically coupled to the substrate by a mechanism that is operable to horizontally slide the second substrate into proximity with the substrate.

29. The method of claim 26, wherein the liquid is caused to flow into a portion of a second the second channel that defines an open-sided, half-pipe configuration.

30. The method of claim 26, wherein holding the liquid within the open-ended microfluidic channel includes holding the substrate in an orientation wherein the lower end is oriented downward and the surface tension holds the liquid against the force of gravity.

31-41. (canceled)

42. A microfluidic system, the system comprising:
a first channel comprising an open end;
an intermediate channel comprising two open ends, wherein the first channel and the intermediate channel are slidable relative to each other such that when one open end of the intermediate channel and the open end of the first channel are aligned, fluid flows from the first channel into the intermediate channel;
a second channel, wherein the second channel is an open channel; and wherein the intermediate and second channels are positioned orthogonal relative to each other and are slidable relative to each other such that when an open end of the intermediate channel and a portion of the second channel are aligned with each other, fluid flows from the intermediate channel into the second channel.

43. (canceled)

44. The microfluidic system according to claim 42, wherein the first and intermediate channels are arranged in relation to each such that an air gap exists when the open end of the first channel and the open end of the intermediate channel are aligned with each other and fluid from the first channel bridges the air gap and enters the intermediate channel.

45-46. (canceled)

47. The microfluidic system according to claim 42, wherein the intermediate and second channels are arranged in relation to each such that an air gap exists when the open end of the intermediate channel and an open portion of the second channel are aligned with each other and fluid from the intermediate channel bridges the air gap and enters the second channel.

48. (canceled)

49. The microfluidic system according to claim 42, wherein the first, intermediate and second channels are arranged in relation to each such that an air gap exists when the open end of the first channel and the open end of the intermediate channel are aligned with each other and such that an air gap exists when the second open end of the intermediate channel and a portion of the second channel are aligned with each other such that fluid from the first channel bridges the air gap and enters the intermediate channel and fluid from the intermediate channel bridges the air gap and enters the second channel.

50. (canceled)

51. A method for handling fluid, the method comprising:
providing a microfluidic system that comprises a first channel comprising an open end;
and a second channel, wherein the second channel is an open channel, wherein the first and second channels are slidable relative to each other such that when the open end of the first channel and the open second channel are aligned with each other, fluid flows from the first channel into the second channel;
loading a fluid into the first channel; and
sliding either the first or second channel such that the open end of the first channel is aligned with the open portion of the open channel, thereby causing at least a portion of the fluid to flow into the second channel.

52. The method according to claim 51, wherein the first and second channels are arranged in relation to each such that an air gap exists when the open end of the first channel and the open portion of the open second channel are aligned with each other and fluid from the first channel bridges the air gap and enters the second channel.

53-55. (canceled)

56. The method according to claim 51, wherein the fluid comprises droplets that are immiscible with the fluid.

57. The method according to claim 56, wherein the fluid comprises an oil and the droplets comprise an aqueous fluid.

58. The method according to claim 57, wherein the fluid further comprises a surfactant.
